(19) United States
(12) Patent Application Publication
(10) Pub. No.: US 2024/0400953 A1
(43) Pub. Date: Dec. 5, 2024

(54) METHODS AND COMPOSITIONS FOR GAMMA-DECALACTONE BIOSYNTHESIS IN FERMENTED BEVERAGES

(71) Applicant: Berkeley Fermentation Science Inc., Oakland, CA (US)

(72) Inventors: Rachel Li, Berkeley, CA (US); Charles Denby, Berkeley, CA (US); Jeremy Roop, Berkeley, CA (US); Nicholas Harris, Glenwood Springs, CO (US); Sara Stadulis, Berkeley, CA (US)

(73) Assignee: Berkeley Fermentation Science Inc., Oakland, CA (US)

(21) Appl. No.: 18/560,836

(22) PCT Filed: May 20, 2022
(86) PCT No.: PCT/US2022/030364

§ 371 Date: Nov. 14, 2023

Related U.S. Application Data
(60) Provisional application No. 63/190,954, filed on May 20, 2021.

(51) Int. Cl. C12C 12/00; C12N 1/18; C12N 9/02; C12N 9/10; C12N 15/81; C12P 7/06; C12P 17/04

(52) U.S. Cl. CPC C12C 12/004; C12C 12/006; C12N 1/18; C12N 9/0071; C12N 9/1029; C12N 15/81; C12P 7/06; C12P 17/04; C12Y 114/18; C12Y 114/19001; C12Y 203/01084

(57) ABSTRACT

Provided herein are genetically modified yeast cells that recombinantly express a gene encoding a fatty acid hydroxylase (FAH) enzyme, such as an oleate 12-hydroylase, and produce y-decalactone levels above an odor-threshold. Also provided herein are genetically modified yeast cells that recombinantly express genes encoding a fatty acid hydroxylase (FAH) enzyme, and one or more additional genes, such as an acyl-CoA desaturase 1 (OLE1) enzyme, a deregulated transcription factor, and/or an alcohol-O-acyltransferase (AAT) enzyme. Also provided are methods of producing fermented beverages and compositions comprising ethanol using the genetically modified yeast cells described herein.

### RELATED APPLICATION

This application claims the benefit under 35 U.S.C. § 119(e) of U.S. Provisional Application No. 63/190,954, filed May 20, 2021, which is incorporated by reference in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on May 20, 2022, is named B150970002WO00-SEQ-CEW.txt, and is 68,582 bytes in size.

### BACKGROUND

Stone fruit flavors, such as peach, nectarine, and apricot, are highly desirable in the beer, wine, and spirit industries. In the wine industry, apricot and peach notes are commonly associated with white wine varietals, especially Chardonnays (Siebert, et al., J. Agric. Food Chem. (2018) 66: 2838-2850; Gambatta, et al., J. Agric. Food Chem. (2014) 62: 6512-6534; Lorrain, et al., J. Agric. Food Chem. (2006) 54: 3973-3981; Lee, et al., J. Agric. Food Chem. (2003) 51: 8036-8044; Siebert, et al., Food Chem. (2018) 256: 286-296), which account for the largest market share of any wine style (Ecker (2019)). In the beer industry, peach flavors can be found in heavily dry-hopped and fruity beers (Hotchko (2014); Holt, et al., FEMS Microbiol. Rev. (2019) 43: 193-222), which have become increasingly popular over the past decade (Watson (2018)). In the context of malt whiskeys, lactones contribute sweet, fruity aromas that drive popularity and perceptions of quality (Wanikawa, et al., Journal of the Institute of Brewing (2000) 106: 39-44; Wanikawa, et al., Journal of the American Society of Brewing Chemists (2000) 58: 51-56).

The stone fruit flavors present in beer, wine, and spirits are predominantly imparted by C6-C12 lactone molecules present in varying concentrations. Among these lactones, γ-decalactone (gamma-decalactone) is a contributor to stone fruit aroma (Wanikawa, et al., Journal of the Institute of Brewing (2000) 106: 39-44; Holt, et al., FEMS Microbiol. Rev. (2019) 43: 193-222; Perez-Olivero, et al., J. Anal. Methods Chem. (2014) 863019; Poisson, et al., J. Agric. Food Chem. (2008) 56: 5813-5819; Wanikawa, et al., Journal of the Institute of Brewing. (2001) 107: 253-259). In isolation, 7-decalactone imparts a strong peach aroma and taste. In combination with other lactones and additional flavor molecules like terpenes and esters, γ-decalactone enhances the complexity of stone fruit and other fruity flavors (Hotchko, et al., J. Am. Soc. Brew. Chem. (2017) 75: 27-34).

### SUMMARY

The present disclosure relates, at least in part, to genetically modified yeast cells capable of biosynthesizing γ-decalactone (gamma-decalactone), and methods of use thereof in producing fermented beverages, such as beer, wine, and spirits, and compositions comprising ethanol.

Aspects of the present disclosure relate to a genetically modified yeast cell (modified cell) comprising a heterologous gene encoding an enzyme having oleate 12-hydroxylase activity; wherein the modified cell is capable of producing a fermented product having an increased level of γ-decalactone in the absence of fatty acid supplementation as compared to a level of γ-decalactone produced by a counterpart cell that does not comprise the enzyme having oleate 12-hydroxylae activity.

Aspects of the present disclosure relate to a genetically modified yeast cell (modified cell) comprising: a heterologous gene encoding an enzyme having oleate 12-hydroxylase activity; wherein the modified cell is capable of producing a fermented product having a level of γ-decalactone greater than 35 μg/L in the absence of fatty acid supplementation.

In some embodiments, the enzyme having oleate 12-hydroxylase activity is from Claviceps purpurea, Lesquerella fendleri, Hiptage benghalensis, Physaria lindheimeri, or Ricinus communis. In some embodiments, the enzyme having oleate 12-hydroxylase activity comprises a sequence having at least 90% sequence identity to the amino acid sequence forth in any one of SEQ ID NOs: 6 or 20-23. In some embodiments, the enzyme having oleate 12-hydroxylase activity comprises the amino acid sequence set forth in any one of SEQ ID NOs: 6 or 20-23. In some embodiments, the enzyme having oleate 12-hydroxylase activity comprises a sequence having at least 90% sequence identity to the amino acid sequence forth in SEQ ID NOs: 6. In some embodiments, the enzyme having oleate 12-hydroxylase activity comprises the amino acid sequence set forth in SEQ ID NOs: 6.

In some embodiments, the modified cell further comprises a gene encoding a deregulated transcription factor that increases peroxisomal size and number and increases beta-oxidation as compared to a counterpart transcription factor that is not deregulated. In some embodiments, the deregulated transcription factor is ADR1, PIP2, OAF1, or OAF3. In some embodiments, the deregulated transcription factor is ADR1 and comprises a substitution mutation of serine at position 230. In some embodiments, the deregulated transcription factor comprises an amino acid sequence having at least 90% sequence identity to the amino acid sequence set forth in SEQ ID NO: 24. In some embodiments, the deregulated transcription factor comprises the amino acid sequence set forth in SEQ ID NO: 24.

In some embodiments, the gene encoding the deregulated transcription factor is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

In some embodiments, the modified cell further comprises a gene encoding an enzyme having acyl-CoA desaturase 1 (OLE1) activity and/or a heterologous gene encoding an enzyme having alcohol-O-acyltransferase (AAT) activity. In some embodiments, the enzyme having OLE1 activity is derived from Saccharomyces cerevisiae. In some embodiments, the enzyme having OLE1 activity comprises a sequence having at least 90% sequence identity to the amino acid sequence forth in SEQ ID NO: 7. In some embodiments, the enzyme having OLE1 activity comprises the amino acid sequence set forth in SEQ ID NO: 7. In some embodiments, the gene encoding the enzyme having acyl-CoA desaturase 1 (OLE1) activity is a copy of an endogenous gene encoding the enzyme having OLE1 activity.

In some embodiments, the enzyme having AAT activity is from Prunus persica, Fragaria x ananassa, Solanum lycopersicum, Malus domestica, or Cucumis melo. In some embodiments, the enzyme having AAT activity comprises a sequence having at least 90% sequence identity to the amino acid sequence set forth in any one of SEQ ID NOs: 1-5 or 25. In some embodiments, the enzyme having AAT activity comprises the amino acid sequence set forth in any one of SEQ ID NOs: 1-5 or 25.

In some embodiments, the gene encoding the enzyme having oleate 12-hydroxylase activity is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

In some embodiments, the gene encoding the deregulated transcription factor, the gene encoding the enzyme having acyl-CoA desaturase 1 (OLE1) activity, and/or the gene encoding the enzyme having alcohol-O-acyltransferase (AAT) activity is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, or Epernay II. In some embodiments, the yeast cell is of the species Saccharomyces pastorianus (S. pastorianus). In some embodiments, growth rate of the modified cell is not substantially impaired relative to a wild-type yeast cell that does not comprise the enzyme having oleate 12-hydroxylase activity.

In some embodiments, within one month of the start of fermentation, the modified cell ferments a comparable amount of fermentable sugar to the amount fermented by wild-type yeast cell that does not comprise the enzyme having oleate 12-hydroxylase activity. In some embodiments, within one month of the start of fermentation, the modified cell reduces the amount of fermentable sugars in a medium by at least 95%. In some embodiments, within one month of the start of fermentation, the modified cell ferments a comparable amount of fermentable sugar to the amount fermented by wild-type yeast cell that does not comprise the enzyme having oleate 12-hydroxylase activity under anaerobic or semi-anaerobic conditions.

Aspects of the present disclosure relate to a genetically modified yeast cells (modified cell) comprising two or more genes, wherein the two or more genes are selected from the group consisting of: a first heterologous gene encoding an enzyme having alcohol-O-acyltransferase (AAT) activity, a second heterologous gene encoding an enzyme having fatty acid hydroxylase (FAH) activity, and a gene encoding an enzyme having acyl-CoA desaturase 1 (OLE1) activity. In some embodiments, the two or more genes of the modified cell comprise the first heterologous gene encoding the enzyme having AAT activity and the second heterologous gene encoding the enzyme having FAH activity. In some embodiments, the two or more genes of the modified cell comprise the second heterologous gene encoding the enzyme having FAH activity and the gene encoding the enzyme having OLE1 activity. In some embodiments, the two or more genes of the modified cell comprise the first heterologous gene encoding the enzyme having AAT activity, the second heterologous gene encoding the enzyme having FAH activity, and the gene encoding the enzyme having OLE1 activity.

In some embodiments, the enzyme having AAT activity is derived from Prunus persica, Fragaria x ananassa, Solanum lycopersicum, Malus domestica, or Cucumis melo. In some embodiments, the enzyme having AAT activity comprises the amino acid sequence set forth in any one of SEQ ID NOs: 1-5 or 25. In some embodiments, the enzyme having AAT activity comprises the amino acid sequence set forth in SEQ ID NO: 1.

In some embodiments, the enzyme having FAH activity is derived from Claviceps purpurea. In some embodiments, the enzyme having FAH activity comprises a sequence having at least 90% sequence identity to the amino acid sequence forth in SEQ ID NO: 6 or 20-23. In some embodiments, the enzyme having FAH activity comprises the amino acid sequence set forth in SEQ ID NO: 6.

In some embodiments, the enzyme having OLE1 activity is derived from Saccharomyces cerevisiae. In some embodiments, the enzyme having OLE1 activity comprises a sequence having at least 90% sequence identity to the amino acid sequence forth in SEQ ID NO: 7. In some embodiments, the enzyme having OLE1 activity comprises the amino acid sequence set forth in SEQ ID NO: 7. In some embodiments, the gene encoding the enzyme having acyl-CoA desaturase 1 (OLE1) activity is a copy of an endogenous gene encoding the enzyme having OLE1 activity.

In some embodiments, each of the genes is operably linked to a promoter selected from the group consisting of pHEM13, pSPG1, pPRB1, pQCR10, pPGK1, pOLE1, pERG25, pHHF2, pTDH1, pTDH2, pTDH3, pENO2, and pHSP26. In some embodiments, at least one of the genes encodes a localization signal linked to the enzyme. In some embodiments, the enzyme having AAT activity comprises a localization signal. In some embodiments, the localization signal is a peroxisome targeting signal.

In some embodiments, the yeast cell is of the genus Saccharomyces. In some embodiments, the yeast cell is of the species Saccharomyces cerevisiae (S. cerevisiae). In some embodiments, the yeast cell is S. cerevisiae California Ale Yeast strain WLP001, EC-1118, Elegance, Red Star Côte des Blancs, or Epernay II. In some embodiments, the yeast cell is of the species Saccharomyces pastorianus (S. pastorianus).

In some embodiments, growth rate of the modified cell is not substantially impaired relative to a wild-type yeast cell that does not comprise the first heterologous gene, the second heterologous gene, and the third gene. In some embodiments, within one month of the start of fermentation, the modified cell ferments a comparable amount of fermentable sugar to the amount fermented by wild-type yeast cell that does not comprise the first heterologous gene, the second heterologous gene, and/or the third gene. In some embodiments, within one month of the start of fermentation, the modified cell reduces the amount of fermentable sugars in a medium by at least 95%. In some embodiments, within one month of the start of fermentation, the modified cell ferments a comparable amount of fermentable sugar to the amount fermented by wild-type yeast cell that does not comprise the first heterologous gene, the second heterologous gene, and the third gene under anaerobic or semi-anaerobic conditions.

In some embodiments, the modified cell further comprises a deregulated transcription factor that increases peroxisomal size and number and increases and beta-oxidation. In some embodiments, the deregulated transcription factor is ADR1, PIP2, OAF1, or OAF3. In some embodiments, the deregulated transcription factor is ADR1 and comprises a substitution mutation of serine at position 230.

Aspects of the present disclosure relate to a method of producing a fermented product comprising, contacting any of the modified cells described herein with a medium comprising at least one fermentable sugar, wherein the contacting is performed during at least a first fermentation process, to produce a fermented product. In some embodiments, the medium does not comprise supplemented fatty acids. In some embodiments, the medium does not comprise supplemented oleic acid and/or ricinoleic acid.

In some embodiments, at least one fermentable sugar is provided in at least one sugar source. In some embodiments, the fermentable sugar is glucose, fructose, sucrose, maltose, and/or maltotriose. In some embodiments, the fermented product comprises an increased level of at least one desired product as compared to a fermented product produced by a counterpart cell that does not express the heterologous gene encoding the enzyme having oleate 12-hydroxylase activity. In some embodiments, the desired product is γ-decalactone. In some embodiments, the fermented product comprises a reduced level of at least one undesired product as compared to a fermented product produced by a counterpart cell that does not express the heterologous gene encoding the enzyme having oleate 12-hydroxylase activity. In some embodiments, the at least one undesired product is ethyl acetate.

In some embodiments, the fermented product is a fermented beverage. In some embodiments, the fermented beverage is beer, wine, sparkling wine (champagne), wine cooler, wine spritzer, hard seltzer, sake, mead, kombucha, or cider. In some embodiments, the sugar source comprises wort, must, fruit juice, honey, rice starch, or a combination thereof. In some embodiments, the sugar source is pre-oxygenated prior to the first fermentation process. In some embodiments, the first fermentation process comprises aeration for a period of time. In some embodiments, the period of time is at least 3 hours.

In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises: (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruit to produce the must. In some embodiments, the method further comprises removing solid fruit material from the must to produce a fruit juice. In some embodiments, the method further comprises at least one additional fermentation process. In some embodiments, the method further comprises carbonating the fermented product.

Aspects of the present disclosure relate to fermented products produced, obtained, or obtainable by any of the methods described herein.

Aspects of the present disclosure relate to methods of producing a composition comprising ethanol, the method comprising contacting any of the modified cells described herein with a medium comprising at least one fermentable sugar, wherein such contacting is performed during at least a first fermentation process, to produce the composition comprising ethanol.

In some embodiments, at least one fermentable sugar is provided in at least one sugar source. In some embodiments, the fermentable sugar is glucose, fructose, sucrose, maltose, and/or maltotriose. In some embodiments, the composition comprising ethanol comprises an increased level of at least one desired product as compared to a composition comprising ethanol produced by a counterpart cell that does not express the heterologous gene encoding an enzyme having oleate 12-hydroxylase activity or a counterpart cell that expresses a wild-type enzyme having oleate 12-hydroxylase activity. In some embodiments, the desired product is γ-decalactone.

In some embodiments, the composition comprising ethanol comprises a reduced level of at least one undesired product as compared to a composition comprising ethanol produced by a counterpart cell that does not express the heterologous gene encoding an enzyme having oleate 12-hydroxylase activity or a counterpart cell that expresses a wild-type enzyme having oleate 12-hydroxylase activity.

In some embodiments, the composition comprising ethanol is a fermented beverage. In some embodiments, the fermented beverage is beer, wine, sparkling wine (champagne), wine cooler, wine spritzer, hard seltzer, sake, mead, kombucha, or cider.

In some embodiments, the sugar source is pre-oxygenated prior to the first fermentation process. In some embodiments, the first fermentation process comprises aeration for a period of time. In some embodiments, the period of time is at least 3 hours.

In some embodiments, the sugar source comprises wort, must, fruit juice, honey, rice starch, or a combination thereof. In some embodiments, the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

In some embodiments, the sugar source is wort and the method further comprises producing the medium, wherein producing the medium comprises: (a) contacting a plurality of grains with water; and (b) boiling or steeping the water and grains to produce wort. In some embodiments, the method further comprises adding at least one hop variety to the wort to produce a hopped wort. In some embodiments, the method further comprises adding at least one hop variety to the medium.

In some embodiments, the sugar source is must and the method further comprises producing the medium, wherein producing the medium comprises crushing a plurality of fruits to produce the must. In some embodiments, the method further comprises removing solid fruit material from the must to produce a fruit juice. In some embodiments, the method further comprises at least one additional fermentation process. In some embodiments, the method further comprises carbonating the composition comprising ethanol.

Aspects of the present disclosure relate to compositions comprising ethanol produced, obtained, or obtainable by any of the methods described herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic showing the spontaneous or enzyme catalyzed synthesis of γ-decalactone from 4-hydroxydecanoic acid (for example, from grapes/barley) in the production of fermented beverages.

FIG. 2 shows a schematic showing a biochemical pathway of γ-decalactone biosynthesis in genetically modified yeast cells described herein.

FIG. 3 shows concentrations of γ-decalactone (μg/L) produced by engineered wine yeast strains following 24 hours of aerobic growth. The parental Saccharomyces cerevisiae Elegance strain was engineered to express the indicated heterologous oleate 12-hydroxylase enzymes under control of the PGK1 promoter. Strains correspond to S. cerevisiae expressing CpFAH (y1094), S. cerevisiae expressing HbFAH (y1330), S. cerevisiae expressing P1FAH (y1331), S. cerevisiae expressing RcFAH (y1332), and S. cerevisiae expressing LFAH12 (y1333). The dashed line corresponds to the odor threshold of γ-decalactone in wine (35 g/L).

FIG. 4 shows concentrations of γ-decalactone (μg/L) produced by engineered beer yeast S. cerevisiae California Ale WLP001 (WLP001) or wine yeast S. cerevisiae Elegance strain after 24 hours of aerobic growth. The parental beer and wine yeast strains were engineered to express the indicated heterologous oleate 12-hydroxylase enzymes under control of the PGK1 promoter. Strains correspond to S. cerevisiae WLP001 expressing LFAH12 (y465), S. cerevisiae Elegance expressing LFAH12 (y1333), S. cerevisiae WLP001 expressing CpFAH (y467), and S. cerevisiae Elegance expressing CpFAH (y1094). The dashed line corresponds to the odor threshold of γ-decalactone in wine (35 μg/L).

FIG. 5 shows concentrations of γ-decalactone (μg/L) produced by engineered yeast strains following 24 hours of aerobic growth. The parental wine yeast S. cerevisiae Elegance strain was engineered to express the indicated heterologous enzymes. Strains correspond to S. cerevisiae expressing CpFAH under control of the PGK1 promoter (y1094); S. cerevisiae expressing CpFAH under control of the PGK1 promoter and OLE1 under control of the ENO2 promoter (y1070); and S. cerevisiae expressing CpFAH under control of the PGK1 promoter, OLE1 under control of the ENO2 promoter, and ADR1(S230A) under control of the RPL18B promoter (y1341). The dashed line corresponds to the odor threshold of γ-decalactone in wine (35 μg/L).

FIG. 6 shows concentrations of γ-decalactone (μg/L) produced by engineered yeast strains after the indicated length of aeration followed by a 9 day fermentation. The parental wine yeast S. cerevisiae Elegance strain was engineered to express CpFAH under control of the TDH3 promoter, OLE1 under control of the ENO2 promoter, MpAAT1 N385D V62A under control of the HSP26 promoter, and ADR1(S230A) under control of the RPL18B promoter (corresponding to strain y1185). Conditions correspond to “anaerobic fermentation” referring to no aerobic growth period; “3 hour aeration” referring to 3 hours of aerobic growth, and “24 hour aeration” referring to 24 hours of aerobic growth prior to a 9 day fermentation.

### DETAILED DESCRIPTION

Stone fruit flavors are highly desirable to consumers in the fermented beverage market. Apricot and peach are especially popular, as evidenced by the robust sales of Chardonnay wines, and beers produced with stone fruit-aroma flavoring hops. The presence of these flavors in both fruits and fermented beverages is due to various flavor-active molecules that collectively impart distinctive tastes and aromas when consumed. One such molecule, γ-decalactone, contributes to many fruity and stone fruit flavors. In isolation, γ-decalactone is perceived as peach, but it also contributes to the flavor of many other fruits (Zhang, et al., Plant Cell Rep. 36, 829-842 (2017)). The modified yeast cells and methods described herein aim to increase concentrations of γ-decalactone produced during fermentation, such as for production of beer or wine. Although several microorganisms are naturally capable of producing γ-decalactone, such as Sporoidiobolus salmonicolor, Fusarium poae, and Ashbya gossypii, these organisms are not used in the production of fermented products, such as fermented beverages.

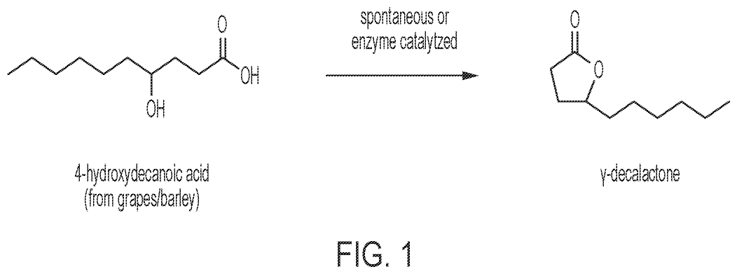

γ-decalactone present in beer, wine, and spirits is thought to originate during fermentation, via the intramolecular esterification of 4-hydroxydecanoic acid that is derived from grapes and barley (FIG. 1). This intramolecular esterification, or “lactonization,” leads to the formation of an oxygen containing lactone ring bound to a six carbon acyl chain. The biochemical details of lactonization during fermentation have not been fully elucidated. It is thought that lactonization can occur spontaneously during fermentation, but it can also be catalyzed by yeast-encoded enzymes (see, e.g., Romero-Guido, et al., Appl. Microbiol. Biotechnol. (2011) 89, 535-547; Krzyczkowska, et al., Fungal Metabolites (2017) 89: 461-498). The relative contributions of these two modes of lactonization to the abundance of γ-decalactone, and lactones in general, in fermented beverages is not known. The extent to which lactone production is limited by the availability of fatty acid precursors, or by the rate of the lactonization reaction itself, is also unknown. However, it is commonly understood that the total concentrations of lactone molecules produced in beer, wine, and other spirits are low, generally in the 1-6 μg/L range (see, e.g., Perez-Olivero, et al., J. Anal. Methods Chem. (2014) 863019; Langen, et al. Rapid Commun. Mass Spectrom. (2013) 27, 2751-2759). As this concentration is well below the threshold of human detection for lactones, lactones (and thus stone fruit flavors) are relatively minor contributors to the overall flavor profiles of most fermented beverages (see, e.g., Perez-Olivero, et al., J. Anal. Methods Chem. (2014) 863019; Cooke, et al., J. Agric. Food Chem. (2009) 57, 2462-2467; Hotchko, et al., J. Amer. Soc. of Brewing Chem. (2017) 75 27-34).

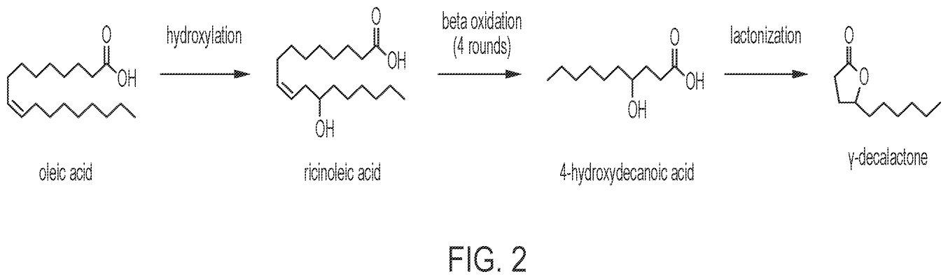

The pathway to produce γ-decalactone begins with oleic acid, a monounsaturated fatty acid containing an 18 carbon chain length (C18) that is produced by both plants and fungal species (FIG. 2). The first step is the hydroxylation of oleic acid at the C12 position to produce ricinoleic acid. Ricinoleic acid is then imported into the peroxisome where it is thought to undergo beta-oxidation, a process that produces cellular acetyl-CoA through progressive shortening of the C18 hydrocarbon chain (see, e.g., Wache, et al., Appl. and Environ. Microbiol. (2001) 67: 5700-5704). While beta-oxidation is capable of oxidizing a C18 ricinoleic acid molecule to produce nine acetyl-CoA molecules, it is 4-hydroxydecanoic acid, a C10 metabolic intermediate that is released after four rounds of ricinoleic acid beta-oxidation, that is relevant to γ-decalactone biosynthesis. 4-hydroxydecanoic acid is the immediate precursor to γ-decalactone. Following its release from beta-oxidation, it can be lactonized to produce γ-decalactone. Without wishing to be bound by any particular theory, it is thought that in plants and fungi, lactonization occurs within the peroxisome or that 4-hydroxydecanoic acid is transported out of the peroxisome and lactonization occurs in the cytoplasm. Efforts to genetically engineer microorganisms to produce γ-decalactone relied on supplementation of the growth medium with fatty acids that are precursors to γ-decalactone production, such as oleic acid and/or ricinoleic acid. See, e.g., Braga et al. World J. Microbiol. Biotechnol. (2016) 32(10): 169.

The modified cells described herein are capable of producing increased levels of γ-decalactone in a medium that has not been supplemented with precursors to γ-decalactone production, such as oleic acid and/or ricinoleic acid. The addition of oleic acid and/or ricinoleic acid to beverage fermentation processes presents several cost and regulatory issues. However, the modified cells described herein do not require supplementation with precursors to γ-decalactone production and are capable of producing levels of γ-decalactone above the odor threshold in wine (i.e., about 35μ/L).

Provided herein are modified yeast cells that have been engineered to express a heterologous gene encoding an enzyme having fatty acid hydroxylase (FAH) activity (e.g., oleate 12-hydroxylase). In some embodiments, the yeast further comprises one or more additional genes, such as a gene encoding a deregulated transcription factor (e.g., ADR1), a gene encoding an enzyme having acyl-CoA desaturase 1 (OLE1) activity, and/or gene encoding an enzyme having alcohol-O-acyltransferase (AAT) activity.

Also provided herein are modified yeast cells that have been engineered to express two or more genes encoding an enzyme having fatty acid hydroxylase (FAH) activity, a deregulated transcription factor, an enzyme having acyl-CoA desaturase 1 (OLE1) activity, and/or an enzyme having alcohol-O-acyltransferase (AAT) activity. In some embodiments, the modified yeasts are used to produce fermented products having increased levels of γ-decalactone. In some embodiments, the modified yeast produce fermented products having decreased levels of ethyl acetate.

In some embodiments, the modified yeast cells express a heterologous gene encoding an enzyme having fatty acid hydroxylase (FAH) activity. In some embodiments, the modified yeast cells express a heterologous gene encoding an enzyme having fatty acid hydroxylase (FAH) activity and an enzyme having alcohol-O-acyltransferase (AAT) activity. In some embodiments, the modified yeast cells express a heterologous gene encoding an enzyme having alcohol-O-acyltransferase (AAT) activity and a gene encoding an enzyme having acyl-CoA desaturase 1 (OLE1) activity. In some embodiments, the modified yeast cells express a heterologous gene encoding an enzyme having fatty acid hydroxylase (FAH) activity and a gene encoding an enzyme having acyl-CoA desaturase 1 (OLE1) activity. In some embodiments, the modified yeast cells express a heterologous gene encoding an enzyme having fatty acid hydroxylase (FAH) activity, a deregulated transcription factor, and a gene encoding an enzyme having acyl-CoA desaturase 1 (OLE1) activity. In some embodiments, the modified yeast cells express a heterologous gene encoding an enzyme having fatty acid hydroxylase (FAH) activity, a deregulated transcription factor, a gene encoding an enzyme having acyl-CoA desaturase 1 (OLE1) activity, and a heterologous gene encoding an enzyme having alcohol-O-acyltransferase (AAT) activity. Also provided herein are methods of producing a fermented beverage involving contacting the modified yeast cells with a medium comprising a sugar source comprising at least one fermentable sugar during a fermentation process. Also provided herein are methods of producing ethanol involving contacting the modified yeast cells with a medium comprising a sugar source comprising at least one fermentable sugar during a fermentation process.

### Fatty Acid Hydroxylase (FAH) Enzymes

The modified cells described herein may contain a gene encoding an enzyme with fatty acid hydroxylase (FAH) activity. In some embodiments, the enzyme with fatty acid hydroxylase (FAH) activity is an oleate 12-hydroxylase (FAH12) enzyme. In some embodiments, the gene is a heterologous gene. In some embodiments, the modified yeast cells express a heterologous gene encoding an enzyme having alcohol-O-acyltransferase (AAT) activity and a heterologous gene encoding an enzyme having fatty acid hydroxylase (FAH) activity. In some embodiments, the modified yeast cells express a heterologous gene encoding an enzyme having fatty acid hydroxylase (FAH) activity and a gene encoding an enzyme having acyl-CoA desaturase 1 (OLE1) activity. In some embodiments, the modified yeast cells express a heterologous gene encoding an enzyme having alcohol-O-acyltransferase (AAT) activity, a heterologous gene encoding an enzyme having fatty acid hydroxylase (FAH) activity, and a gene encoding an enzyme having acyl-CoA desaturase 1 (OLE1) activity.

In some embodiments, the modified yeast cells express a heterologous gene encoding an enzyme having fatty acid hydroxylase (FAH) activity and a deregulated transcription factor, such as ADR (e.g., ADR S230A). In some embodiments, the modified yeast cells express a heterologous gene encoding an enzyme having fatty acid hydroxylase (FAH) activity, a gene encoding an enzyme having acyl-CoA desaturase 1 (OLE1) activity, and a gene encoding a deregulated transcription factor, such as ADR (e.g., ADR S230A). In some embodiments, the modified yeast cells express a heterologous gene encoding an enzyme having fatty acid hydroxylase (FAH) activity, a gene encoding an enzyme having acyl-CoA desaturase 1 (OLE1) activity, a heterologous gene encoding an enzyme having alcohol-O-acyltransferase (AAT) activity, and a gene encoding a deregulated transcription factor, such as ADR (e.g., ADR S230A).

Fatty acid hydroxylases are enzymes that catalyze the hydroxylation of fatty acids to produce hydroxy fatty acids. Oleate 12-hydroxylase enzymes can convert oleic acid to ricinoleic acid, a critical step in the biosynthesis of γ-decalactone from oleic acid. In some embodiments, the heterologous gene encoding an enzyme with fatty acid hydroxylase activity is a wild-type fatty acid hydroxylase gene (e.g., a gene isolated from an organism), such as a wild-type oleate 12-hydroxylase enzyme. In some embodiments, the yeast expressing the heterologous gene encoding the enzyme with fatty acid hydroxylase activity is capable of producing increased levels of γ-decalactone in the absence of supplementation of intermediate molecules in the γ-decalactone biosynthesis pathway (e.g., oleic acid, ricinoleic acid).

In some embodiments, the modified yeast cells express a heterologous gene encoding an enzyme having oleate 12-hydroxylase activity. In some embodiments, the modified yeast cells express a heterologous gene encoding an enzyme having oleate 12-hydroxylase activity and a heterologous gene encoding an enzyme having alcohol-O-acyltransferase (AAT) activity. In some embodiments, the modified yeast cells express a heterologous gene encoding an enzyme having oleate 12-hydroxylase activity, and a gene encoding an enzyme having acyl-CoA desaturase 1 (OLE1) activity. In some embodiments, the modified yeast cells express a heterologous gene encoding an enzyme having oleate 12-hydroxylase activity, a heterologous gene encoding an enzyme having alcohol-O-acyltransferase (AAT) activity, and a gene encoding an enzyme having acyl-CoA desaturase 1 (OLE1) activity.

In some embodiments, the modified yeast cells express a heterologous gene encoding an enzyme having oleate 12-hydroxylase activity and a deregulated transcription factor, such as ADR (e.g., ADR S230A). In some embodiments, the modified yeast cells express a heterologous gene encoding an enzyme having oleate 12-hydroxylase activity, a gene encoding an enzyme having acyl-CoA desaturase 1 (OLE1) activity, and a gene encoding a deregulated transcription factor, such as ADR (e.g., ADR S230A). In some embodiments, the modified yeast cells express a heterologous gene encoding an enzyme having oleate 12-hydroxylase activity, a gene encoding an enzyme having acyl-CoA desaturase 1 (OLE1) activity, a heterologous gene encoding an enzyme having alcohol-O-acyltransferase (AAT) activity, and a gene encoding a deregulated transcription factor, such as ADR (e.g., ADR S230A).

In some embodiments, the heterologous gene encoding an enzyme with fatty acid hydroxylase activity is a wild-type fatty acid hydroxylase gene (e.g., a gene isolated from an organism). In some embodiments, the fatty acid hydroxylase is obtained from a bacterium, a fungus, or a plant. In some embodiments, the fatty acid hydroxylase is obtained from a fungus. In some embodiments, the fatty acid hydroxylase is obtained from Claviceps purpurea.

An exemplary enzyme having fatty acid hydroxylase activity is from Claviceps purpurea. The Claviceps purpurea FAH is provided by the amino acid sequence set forth by SEQ ID NO: 6, which corresponds UniProtKB Accession No. B4YQU.1.

(SEQ ID NO: 6)
MASATPAMSENAVLRHKAASTTGIDYESSAAVSPAESPRTSASSTSLSS
LSSLDANEKKDEYAGLLDTYGNAFTPPDFSIKDIRAAIPKHCYERSTIK
SYAYVLRDLLCLSTTFYLFHNFVTPENIPSNPLRFVLWSIYTVLQGLFA
TGLWVIGHECGHCAFSPSPFISDLTGWVIHSALLVPYFSWKFSHSAHHK
GIGNMERDMVFLPRTREQQATRLGRAVEELGDLCEETPIYTALHLVGKQ
LIGWPSYLMTNATGHNFHERQREGRGKGKKNGFGGGVNHFDPRSPIFEA
RQAKYIVLSDIGLGLAIAALVYLGNRFGWANMAVWYFLPYLWVNHWLVA
ITFLQHTDPTLPHYNREEWNFVRGGACTIDRDLGFIGRHLFHGIADTHV
VHHYVSRIPFYNADEASEAIKPIMGKHYRSDTAHGPVGFLHALWKTARW
CQWVEPSADAQGAGKGILFYRNRNKLGTKPISMKTQ*

In some embodiments, the fatty acid hydroxylase is obtained from a plant. In some embodiments, the fatty acid hydroxylase is obtained from Hiptage benghalensis. An exemplary enzyme having fatty acid hydroxylase activity is from Hiptage benghalensis. The Hiptage benghalensis FAH (HbFAH) is provided by the amino acid sequence set forth by SEQ ID NO: 20, which corresponds to GenBank No. KC533768.1; UniProtKB Accession No. R9WAV0.

(SEQ ID NO: 20)
MGAGGRMPTSVSKGQGMENEVKHGPCEKPPFTVGQLKRAIPPHCFERSL
IRSSSYLLRDLFFVFVFYYVATSYFHLLPYPFNYAAWPIYWGFQGCALT
GIWVLGHECGHHAFSDYQLVDDIVGLIIHTALLVPYFSWKISHRRHHSN
TGSLEREEVFAPKPKAEIQWYLKHLNNPPGRAIVLLNTLLLGWPLYVAF
NVAGRRYDRFACHFDPYSPIFSASERHLIYITDAGIYATTFILYRAAAA
KGLTWLICVYGVPLVIVNAFLVLVTYLQHTHPVLPHYDNSEWDWLRGAL
VTVDRDYGILNEVFHHIADTHVAHHLFSKIPQYHGMEATKAIKPILGEY
YQFDGTPFLKALWREARECVYVDRDEGDPKRGVYWYGNKF*

An exemplary enzyme having fatty acid hydroxylase activity is from Physaria lindheimeri. The Physaria lindheimeri FAH (PlFAH) is provided by the amino acid sequence set forth by SEQ ID NO: 21, which corresponds to GenBank No. EF432246.1; UniProtKB Accession No. A51B93.

(SEQ ID NO: 21)
MGAGGRIMVTPSSKKSKPEALRRGPGEKPPFTVQDLRKAIPRHCFKRSI
PRSFSYLLTDIILASCFYYVATNYFSLLPQPLSTYFAWPLYWVCQGCVL
TGVWVLGHECGHQAFSDYQWVDDTVGFIIHTFLLVPYFSWKYSHRRHHA
NNGSLERDEVFVPPKKAAVKWYVKYLNNPLGRTVVLIVQFVLGWPLYLA
FNVSGRSYDGFASHFFPHAPIFKDRERLHIYITDAGILAVCYGLYRYAA
TKGLTAMIYVYGVPLLVVNFFLVLVTFLQHTHPSLPHYDSTEWDWIRGA
MVTVDRDYGILNKVFHNITDTHVAHHLFATIPHYNAMEATEAIKPILGD
YYHFDGTPWYVAMYREAKQCLYVEQDTEKKKGVYYYNNKL*

An exemplary enzyme having fatty acid hydroxylase activity is from Ricinus communis. The Ricinus communis FAH (RcFAH) is provided by the amino acid sequence set forth by SEQ ID NO: 22, which corresponds to GenBank No. U22378.1; UniProtKB Accession No. Q41131.

(SEQ ID NO: 22)
MGGGGRMSTVITSNNSEKKGGSSHLKRAPHTKPPFTLGDLKRAIPPHCF
ERSFVRSFSYVAYDVCLSFLFYSIATNFFPYISSPLSYVAWLVYWLFQG
CILTGLWVIGHECGHHAFSEYQLADDIVGLIVHSALLVPYFSWKYSHRR
HHSNIGSLERDEVFVPKSKSKISWYSKYSNNPPGRVLTLAATLLLGWPL
YLAFNVSGRPYDRFACHYDPYGPIFSERERLQIYIADLGIFATTFVLYQ
ATMAKGLAWVMRIYGVPLLIVNCFLVMITYLQHTHPAIPRYGSSEWDWL
RGAMVTVDRDYGVLNKVFHNIADTHVAHHLFATVPHYHAMEATKAIKPI
MGEYYRYDGTPFYKALWREAKECLFVEPDEGAPTQGVFWYRNKY*

An exemplary enzyme having fatty acid hydroxylase activity from Lesquerella fendleri. The Lesquerella fenderia FAH (LFAH12) is provided by the amino acid sequence set forth by SEQ ID NO: 23, which corresponds to GenBank No. AF016103; UniProtKB Accession No. 081094.

(SEQ ID NO: 23)
MGAGGRIMVTPSSKKSETEALKRGPCEKPPFTVKDLKKAIPQHCFKRSI
PRSFSYLLTDITLVSCFYYVATNYFSLLPQPLSTYLAWPLYWVCQGCVL
TGIWVIGHECGHHAFSDYQWVDDTVGFIFHSFLLVPYFSWKYSHRRHHS
NNGSLEKDEVFVPPKKAAVKWYVKYLNNPLGRILVLTVQFILGWPLYLA
FNVSGRPYDGFASHFFPHAPIFKDRERLQIYISDAGILAVCYGLYRYAA
SQGLTAMICVYGVPLLIVNFFLVLVTFLQHTHPSLPHYDSTEWEWIRGA
LVTVDRDYGILNKVFHNITDTHVAHHLFATIPHYNAMEATEAIKPILGD
YYHFDGTPWYVAMYREAKECLYVEPDTERGKKGVYYYNNKL*

In some embodiments, the heterologous gene encodes an enzyme with fatty acid hydroxylase activity such that a cell that expresses the enzyme is capable of increased production of γ-decalactone as compared to a cell that does not express the heterologous gene.

In some embodiments, the enzyme with fatty acid hydroxylase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any of SEQ ID NOs: 6 or 20-23.

In some embodiments, the enzyme with fatty acid hydroxylase activity comprises the amino acid sequence as set forth in SEQ ID NO: 6. In some embodiments, the enzyme with fatty acid hydroxylase activity consists of the amino acid sequence as set forth in any of SEQ ID NO: 6 or 20-23.

In some embodiments, the gene encoding the enzyme with fatty acid hydroxylase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in any of SEQ ID NO: 6 or 20-23. In some embodiments, the gene encoding the enzyme with fatty acid hydroxylase activity comprises a nucleic acid sequence which encodes an enzyme consisting of an amino acid sequence as set forth in any of SEQ ID NOs: 6 or 20-23.

In some embodiments, the enzyme with fatty acid hydroxylase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in SEQ ID NO: 6.

In some embodiments, the enzyme with fatty acid hydroxylase activity comprises the amino acid sequence as set forth in SEQ ID NO: 6. In some embodiments, the enzyme with fatty acid hydroxylase activity consists of the amino acid sequence as set forth in SEQ ID NO: 6.

In some embodiments, the gene encoding the enzyme with fatty acid hydroxylase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in SEQ ID NO: 6. In some embodiments, the gene encoding the enzyme with fatty acid hydroxylase activity comprises a nucleic acid sequence which encodes an enzyme consisting of an amino acid sequence as set forth in SEQ ID NO: 6.

Identification of additional enzymes having fatty acid hydroxylase activity or predicted to have fatty acid hydroxylase activity may be performed, for example based on similarity or homology with one or more domains of a fatty acid hydroxylase, such as the fatty acid hydroxylase provided by any of SEQ ID NOs: 6 or 20-23 such as SEQ ID NO: 6. In some embodiments, an enzyme for use in the modified cells and methods described herein may be identified based on similarity or homology with an active domain, such as a catalytic domain, such as a catalytic domain associated with fatty acid hydroxylase activity. In some embodiments, an enzyme for use in the modified cells and methods described herein may have a relatively high level of sequence identity with a reference fatty acid hydroxylase, e.g., a wild-type fatty acid hydroxylase, such as any of SEQ ID NOs: 6 or 20-23, in the region of the catalytic domain but a relatively low level of sequence identity to the reference fatty acid hydroxylase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference fatty acid hydroxylase (e.g., any of SEQ ID NOs: 6 or 20-23, e.g., SEQ ID NO: 6).

In some embodiments, an enzyme for use in the modified cells and methods described herein have a relatively high level of sequence identity in the region of the catalytic domain of the enzyme relative to a reference fatty acid hydroxylase (e.g., any of SEQ ID NOs: 6 or 20-23, such as SEQ ID NO: 6) and a relatively low level of sequence identity to the reference alcohol-O-acyltransferase based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference fatty acid hydroxylase (e.g., any of SEQ ID NOs: 6 or 20-23, such as SEQ ID NO: 6).

### Deregulated Transcription Factors

In some embodiments, production of γ-decalactone is increased by genetic modification involving upregulating beta-oxidation, for example by increasing peroxisome size and number. Yeast grown in the presence of excess fatty acids increase peroxisome size and number, and subsequently upregulate beta-oxidation through regulation of several transcription factors, such as ADR1, PIP2, OAF1, and/or OAF3. In some embodiments, the genetically modified cells described herein express or overexpress a gene encoding a transcription factor that promotes peroxisome biogenesis and organization, including increasing peroxisome proliferation and/or increases fatty acid beta-oxidation in the cell, for example as compared to a cell that does not express the transcription factor. In some embodiments, the genetically modified cells described herein comprise a deregulated transcription factor, such as ADR1, PIP2, OAF1, and/or OAF3.

In some embodiments, the modified yeast cells express a heterologous gene encoding an enzyme having fatty acid hydroxylase (FAH) activity and a gene encoding a deregulated transcription factor, such as ADR (e.g., ADR S230A). In some embodiments, the modified yeast cells express a heterologous gene encoding an enzyme having fatty acid hydroxylase (FAH) activity, a gene encoding a deregulated transcription factor, such as ADR (e.g., ADR S230A), and a gene encoding an enzyme having acyl-CoA desaturase 1 (OLE1) activity. In some embodiments, the modified yeast cells express a heterologous gene encoding an enzyme having fatty acid hydroxylase (FAH) activity, a gene encoding a deregulated transcription factor, such as ADR (e.g., ADR S230A), a gene encoding an enzyme having acyl-CoA desaturase 1 (OLE1) activity, and a heterologous gene encoding an enzyme having alcohol-O-acyltransferase (AAT) activity.

One such transcription factor, ADR1, encodes a zinc-finger transcription factor that is repressed through phosphorylation at a serine residue (i.e., serine at position 230 (Ser230)). Yeast grown in the presence of excess fatty acids are thought to activate ADR1 by dephosphorylation of the serine residue. Mutation of the serine residue, for example to an alanine, results in constitutive activation of ADR1, leading to peroxisome proliferation and upregulated beta-oxidation in the absence of fatty acids, such as in a medium containing fermentable sugars. In some embodiments, the genetically modified cells described herein comprise a deregulated ADR1 transcription factor. In some embodiments, the ADR1 transcription factor may be mutated to produce a constitutively active ADR1 transcription factor. In some embodiments, constitutive activity of the ADR1 transcription factor results in peroxisome proliferation and upregulation of beta-oxidation. In some embodiments, the genetically modified cells described herein comprise a deregulated ADR1 transcription factor comprising a substitution mutation of the serine residue at position 230 (Ser230). In some embodiments, the serine residue at position 230 (or corresponding to position 230) is substituted with an alanine residue.

An exemplary deregulated transcription factor is ADR1 from S. cerevisiae, in which the serine residue at position 230 (Ser230, S230) is substituted with an alanine residue (ADR1(S230A)), which is provided by the amino acid sequence set forth in SEQ ID NO: 24.

(SEQ ID NO: 24)
MANVEKPNDCSGFPVVDLNSCFSNGFNNGKQEIEMETDDSPILLMSSSASRENSNTFSVIQRTPDGKIITTNNNM
NSKINKQLDKLPENLRLNGRTPSGKLRSFVCEVCTRAFARQEHLKRHYRSHTNEKPYPCGLCNRCFTRRDLLIRH
AQKIHSGNLGETISHTKKVSRTITKARKNSASSVKFQTPTYGTPDNGNFLNRTTANTRRKASPEANVKRKYLKKL
TRRAAFSAQSASSYALPDQSSLEQHPKDRVKFSTPELVPLDLKNPELDSSFDLNMNLDLNLNLDSNFNIALNRSD
SSGSTMNLDYKLPESANNYTYSSGSPTRAYVGANTNSKNASFSDADLLSSSYWIKAYNDHLFSVSESDETSPMNS
ELNDTKLIVPDFKSTIHHLKDSRSSSWTVAIDNNSNNNKVSDNQPDFVDFQELLDNDTLGNDLLETTAVLKEFEL
LHDDSVSATATSNEIDLSHLNLSNSPISPHKLIYKNKEGTNDDMLISFGLDHPSNREDDLDKLCNMTRDVQAIFS
QYLKGEESKRSLEDFLSTSNRKEKPDSGNYTFYGLDCLTLSKISRALPASTVNNKQPSHSIESKLFNEPMRNMCI
KVLRYYEKFSHDSSESVMDSNPNLLSKELLMPAVSELNEYLDLFKNNFLPHFPIIHPSLLDLDLDSLQRYTNEDG
YDDAENAQLFDRLSQGTDKEYDYEHYQILSISKIVCLPLFMATFGSLHKFGYKSQTIELYEMSRRILHSFLETKR
RCRSTTVNDNYQNIWLMQSLILSFMFALVADYLEKIDSSLMKRQLSALCSTIRSNCLPTISANSEKSINNNNEPL
TFGSPLQYIIFESKIRCTLMAYDFCQFLKCFFHIKFDLSIKEKDVETIYIPDNESKWASESIICNGHVVQKQNFY
DFRNFYYSFTYGHLHSIPEFLGSSMIYYEYDLRKGTKSHVFLDRIDTKRLERSLDTSSYGNDNMAATNKNIAILI
DDTIILKNNLMSMRFIKQIDRSFTEKVRKGQIAKIYDSFLNSARLNFLKNYSVEVLCEFLVALNFSIRNISSLYV
EEESDCSQRMNSPELPRIHLNNQALSVFNLQGYYYCFILIIKFLLDFEATPNFKLLRIFIELRSLANSILLPTLS
RLYPQEFSGFPDVVFTQQFINKDNGMLVPGLSANEHHNGASAAVKTKLAKKINVEGLAMFINEILVNSFNDTSFL
NMEDPIRNEFSFDNGDRAVTDLPRSAHFLSDTGLEGINFSGLNDSHQTVSTLNLLRYGENHSSKHKNGGKGQGFA
EKYQLSLKYVTIAKLFFTNVKENYIHCHMLDKMASDFHTLENHLKGNS*

Alternatively or in addition, the genetically modified cells described herein may comprise a deregulated PIP2 and/or OAF1 transcription factor. In some embodiments, the PIP2 transcription factor and/or the OAF1 transcription factor are mutated to deregulate transcription factor activity, resulting in constitutive activity of the transcription factor. In some embodiments, deregulation of PIP2 and/or OAF1 transcription factor activity results in peroxisome proliferation and upregulation of beta-oxidation.

Alternatively or in addition, the genetically modified cells described herein may comprise a genetic modification to delete (e.g., knockout), reduce expression (e.g., knock down), and/or downregulate the transcriptional repressor OAF3. In some embodiments, the OAF3 transcriptional repressor is mutated to decrease or downregulate transcriptional repressor activity. In some embodiments, the decrease or downregulation of OAF3 transcriptional repressor activity results in peroxisome proliferation and upregulation of beta-oxidation.

Mutation of a nucleic acid sequence encoding a transcription factor, such as ADR1, PIP2, OAF1, and/or OAF3, preferably preserves the amino acid reading frame of the coding sequence, and preferably does not create regions in the nucleic acid which are likely to hybridize to form secondary structures, such a hairpins or loops, which can be deleterious to expression of the enzyme.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon optimization). The preferred codons for translation of a nucleic acid in, e.g., S. cerevisiae, are well known to those of ordinary skill in the art. Still other mutations can be made to the noncoding sequences of a gene or cDNA clone to enhance expression of the polypeptide. The activity of an ADR1 transcription factor, a PIP2 transcription factor, an OAF1 transcription factor, or an OAF3 transcriptional repressor variant can be tested by cloning the gene encoding the enzyme variant into an expression vector, introducing the vector into an appropriate host cell, expressing the enzyme variant, and testing for a functional capability of the enzyme, as disclosed herein.

### Acyl-CoA Desaturase 1 (OLE1) Enzymes

The modified cells described herein may contain a gene encoding an enzyme with acyl-CoA desaturase 1 (OLE1) activity. In some embodiments, the gene is a copy of an endogenous gene encoding an enzyme having OLE1 activity. The term “endogenous gene,” as used herein, refers to a hereditary unit corresponding to a sequence of nucleic acid (e.g., DNA) that contains the genetic instruction, which originates within a host organism (e.g., a genetically modified cell) and is expressed by the host organism.

In some embodiments, the modified yeast cells express a heterologous gene encoding an enzyme having fatty acid hydroxylase (FAH) activity, a gene encoding an enzyme having acyl-CoA desaturase 1 (OLE1) activity, and a gene encoding a deregulated transcription factor, such as ADR1 (e.g., ADR1 S230A). In some embodiments, the modified yeast cells express a heterologous gene encoding an enzyme having fatty acid hydroxylase (FAH) activity, a gene encoding an enzyme having acyl-CoA desaturase 1 (OLE1) activity, a gene encoding a deregulated transcription factor, such as ADR (e.g., ADR S230A), and a heterologous gene encoding an enzyme having alcohol-O-acyltransferase (AAT) activity.

In some embodiments, the modified yeast cells express a heterologous gene encoding an enzyme having alcohol-O-acyltransferase (AAT) activity and a gene encoding an enzyme having acyl-CoA desaturase 1 (OLE1) activity. In some embodiments, the modified yeast cells express a heterologous gene encoding an enzyme having fatty acid hydroxylase (FAH) activity and a gene encoding an enzyme having acyl-CoA desaturase 1 (OLE1) activity. In some embodiments, the modified yeast cells express a heterologous gene encoding an enzyme having alcohol-O-acyltransferase (AAT) activity, a heterologous gene encoding an enzyme having fatty acid hydroxylase (FAH) activity, and a gene encoding an enzyme having acyl-CoA desaturase 1 (OLE1) activity.

Acyl-CoA desaturase 1 enzymes are enzymes that catalyze the conversion of stearic acid to oleic acid and may also be referred to as a stearoyl-CoA 9-desaturases. In some embodiments, oleic acid produced by the acyl-CoA desaturase I activity is used for the production of γ-decalactone, and precursors thereof. In some embodiments, the heterologous gene encoding an enzyme with acyl-CoA desaturase 1 activity is a wild-type acyl-CoA desaturase 1 gene (e.g., a gene isolated from an organism). In some embodiments, the gene encoding the acyl-CoA desaturase 1 is obtained from the fungus belonging to the genus Saccharomyces. In some embodiments, the gene encoding the acyl-CoA desaturase 1 is obtained from the fungus Saccharomyces cerevisiae. In some embodiments, the gene encoding the acyl-CoA desaturase 1 is obtained from the fungus Saccharomyces pastorianus.

An exemplary enzyme having acyl-CoA desaturase 1 activity is OLE1 from Saccharomyces cerevisiae. The Saccharomyces cerevisiae OLE1 is provided by the amino acid sequence set forth by SEQ ID NO: 7, which corresponds to UniProtKB Accession No. AAA34826.1.

(SEQ ID NO: 7)
MPTSGTTIELIDDQFPKDDSASSGIVDEVDLTEANILATGLNKKAPRIV
NGFGSLMGSKEMVSVEFDKKGNEKKSNLDRLLEKDNQEKEEAKTKIHIS
EQPWTLNNWHQHLNWLNMVLVCGMPMIGWYFALSGKVPLHLNVFLFSVF
YYAVGGVSITAGYHRLWSHRSYSAHWPLRLFYAIFGCASVEGSAKWWGH
SHRIHHRYTDTLRDPYDARRGLWYSHMGWMLLKPNPKYKARADITDMTD
DWTIRFQHRHYILLMLLTAFVIPTLICGYFFNDYMGGLIYAGFIRVFVI
QQATFCINSMAHYIGTQPFDDRRTPRDNWITAIVTFGEGYHNFHHEFPT
DYRNAIKWYQYDPTKVIIYLTSLVGLAYDLKKFSQNAIEEALIQQEQKK
INKKKAKINWGPVLTDLPMWDKQTFLAKSKENKGLVIISGIVHDVSGYI
SEHPGGETLIKTALGKDATKAFSGGVYRHSNAAQNVLADMRVAVIKESK
NSAIRMASKRGEIYETGKFF*

In some embodiments, the gene encodes an enzyme with acyl-CoA desaturase 1 activity such that a cell that expresses the enzyme is capable of increased production of γ-decalactone as compared to a cell that does not express the gene or only expresses one copy of the gene. In some embodiments, the gene encodes an enzyme with acyl-CoA desaturase 1 activity such that a cell that expresses the enzyme is capable of producing increased levels of γ-decalactone as compared to a cell that expresses an enzyme with wild-type acyl-CoA desaturase 1 activity.

In some embodiments, the enzyme with acyl-CoA desaturase 1 activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any one of SEQ ID NO: 7.

In some embodiments, the enzyme with acyl-CoA desaturase 1 activity comprises the amino acid sequence as set forth in SEQ ID NO: 7. In some embodiments, the enzyme with acyl-CoA desaturase 1 activity consists of the amino acid sequence as set forth in SEQ ID NO: 7.

In some embodiments, the gene encoding the enzyme with acyl-CoA desaturase 1 activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in SEQ ID NO: 7. In some embodiments, the gene encoding the enzyme with acyl-CoA desaturase 1 activity comprises a nucleic acid sequence which encodes an enzyme consisting of an amino acid sequence as set forth in SEQ ID NO: 7.

Identification of additional enzymes having acyl-CoA desaturase 1 activity or predicted to have acyl-CoA desaturase 1 activity may be performed, for example based on similarity or homology with one or more domains of an acyl-CoA desaturase 1, such as the acyl-CoA desaturase 1 provided by SEQ ID NO: 7. In some embodiments, an enzyme for use in the modified cells and methods described herein may be identified based on similarity or homology with an active domain, such as a catalytic domain, such as a catalytic domain associated with acyl-CoA desaturase 1 activity. In some embodiments, an enzyme for use in the modified cells and methods described herein may have a relatively high level of sequence identity with a reference alcohol-O-acyltransferase, e.g., a wild-type acyl-CoA desaturase 1, such as SEQ ID NO: 7, in the region of the catalytic domain but a relatively low level of sequence identity to the reference acyl-CoA desaturase 1 based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference acyl-CoA desaturase 1 (e.g., SEQ ID NO: 7).

In some embodiments, an enzyme for use in the modified cells and methods described herein has a relatively high level of sequence identity in the region of the catalytic domain of the enzyme relative to a reference acyl-CoA desaturase 1 (e.g., SEQ ID NO: 7) and a relatively low level of sequence identity to the reference acyl-CoA desaturase 1 based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 9.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference acyl-CoA desaturase 1 (e.g., SEQ ID NO: 7).

### Alcohol-O-Acyltransferase (AAT) Enzymes

The modified cells described herein may contain a gene encoding an enzyme with alcohol-O-acyltransferase (AAT) activity. In some embodiments, the gene is a heterologous gene. The term “heterologous gene,” as used herein, refers to a sequence of nucleic acid (e.g., DNA) that contains the genetic instruction, which is introduced into and expressed by a host organism (e.g., a genetically modified cell) which does not naturally encode the introduced gene. The heterologous gene may encode an enzyme that is not typically expressed by the cell, a variant of an enzyme that the cell does not typically express (e.g., a mutated enzyme), an additional copy of a gene encoding an enzyme that is typically expressed in the cell, or a gene encoding an enzyme that is typically expressed by the cell but under different regulation. In some embodiments, the modified yeast cells express a heterologous gene encoding an enzyme having alcohol-O-acyltransferase (AAT) activity and a heterologous gene encoding an enzyme having fatty acid hydroxylase (FAH) activity. In some embodiments, the modified yeast cells express a heterologous gene encoding an enzyme having alcohol-O-acyltransferase (AAT) activity and a gene encoding an enzyme having acyl-CoA desaturase 1 (OLE1) activity. In some embodiments, the modified yeast cells express a heterologous gene encoding an enzyme having alcohol-O-acyltransferase (AAT) activity, a heterologous gene encoding an enzyme having fatty acid hydroxylase (FAH) activity, and a gene encoding an enzyme having acyl-CoA desaturase 1 (OLE1) activity. In some embodiments, the modified yeast cell does not express a gene encoding an enzyme having alcohol-O-acyltransferase (AAT) activity.

Alcohol-O-acyltransferases, which may also be referred to as acetyl-CoA:acetyltransferases or alcohol acetyltransferases, are bisubstrate enzymes that catalyze the transfer of acyl chains from an acyl-coenzyme A (CoA) donor to an acceptor alcohol, resulting in the production of an acyl ester. The acyl esters present in a fermented beverage influence its flavor. The ester γ-decalactone, which is formed by the lactonization of 4-hydroxydecanoic acid, imparts a peach flavor to fermented beverages such as beer and wine.

In some embodiments, the heterologous gene encoding an enzyme with alcohol-O-acyltransferase activity is a wild-type alcohol-O-acyltransferase gene (e.g., a gene isolated from an organism). In some embodiments, the alcohol-O-acyltransferase is obtained from a bacterium or a fungus.

In some embodiments, the alcohol-O-acyltransferase is obtained from a plant, such as crop plant. In some embodiments, the alcohol-O-acyltransferase is obtained from a peach plant. In some embodiments, the alcohol-O-acyltransferase gene is from Prunus persica.

An exemplary enzyme having alcohol-O-acyltransferase activity is PpAAT1 from Prunus persica. The Prunus persica AAT is provided by the amino acid sequence set forth as SEQ ID NO: 1, which corresponds to UniProtKB Accession No. XP_007209131.1.

(SEQ ID NO: 1)
MGSLCPLLFPVNRFEPELITPAKPTPIETKQLSDIDDQDGLRFHFPVII
SYKNNPSMKGNDAVMVIREALSRALVYYYPLAGRLREGPNRKLMVECNG
EGVLFIEANADVTLEQLGDRILPPCPVLEEFLSNPPGSDGILGCPLLLV
QVTRLTCGGFIFGLRINHAMCDAVGLAKFLNAIGEMAQGADSLSVPPVW
ARELLNARDPPTVTRWHYEYDQLLDSQGSFIAAIDQSNMAQRSFYFGPQ
QIRALRKHLPPHLSTCSSFELITACVWRCRTLSLRLNPKDTVRISCAVN
ARGKSINDLCLPSGFYGNAFSIPTAVSTVELLCASPLGYGVELVRKSKA
QMDKEYMQSLADFFVIRGRPPLPMGWNVFIVSDNRHTGFGEFDVGWGRP
LFAGLARAFSMISFYVRDNNQEEEFGTLVPICLPSTSLERFEEELKKMT
LEHVEEISK*

In some embodiments, the enzyme having alcohol-O-acyltransferase activity is SAAT from Fragaria x ananassa. The Fragaria x ananassa AAT is provided by the amino acid sequence set forth as SEQ ID NO: 2, which corresponds to UniProtKB Accession No. AAG13130.1.

(SEQ ID NO: 2)
MGEKIEVSINSKHTIKPSTSSTPLQPYKLTLLDQLTPPAYVPIVFFYPI
TDHDFNLPQTAADLRQALSETLTLYYPLSGRVKNNLYIDDFEEGVPYLE
ARVNCDMTDFLRLRKIECLNEFVPIKPFSMEAISDERYPLLGVQVNVFD
SGIAIGVSVSHKLIDGGTADCFLKSWGAVFRGCRENIIHPSLSEAALLF
PPRDDLPEKYVDQMEALWFAGKKVATRRFVFGVKAISSIQDEAKSESVP
KPSRVHAVTGFLWKHLIAASRALTSGTTSTRLSIAAQAVNLRTRMNMET
VLDNATGNLFWWAQAILELSHTTPEISDLKLCDLVNLLNGSVKQCNGDY
FETFKGKEGYGRMCEYLDFQRTMSSMEPAPDIYLFSSWTNFFNPLDFGW
GRTSWIGVAGKIESASCKFIILVPTQCGSGIEAWVNLEEEKMAMLEQDP
HFLALASPKTLI

In some embodiments, the enzyme having alcohol-O-acyltransferase activity is SpAAT1 from Solanum lycopersicum. The Solanum lycopersicum AAT is provided by the amino acid sequence set forth as SEQ ID NO: 3, which corresponds to UniProtKB Accession No. NP_001310384.1.

(SEQ ID NO: 3)
MANTLPISINYHKPKLVVPSSVTPHETKRLSEIDDQGFIRFQIPILMFY
KYNSSMKGKDPARIIEDGLSKTLVFYHPLAGRLIEGPNKKLMVNCNGEG
VLFIEGDANIELEKLGESIKPPCPYLDLLLHNVPGSDGIIGSPLLLIQV
TRFTCGGFAVGFRVSHTMMDGYGFKMFLNALSELIQGASTPSILPVWQR
HLLSARSSPCITCSHHEFDEEIESKIAWESMEDKLIQESFFFGNEEMEV
IKNQIPPNYGCTKFELLMAFLWKCRTIALDLHPEEIVRLTYVINIRGKK
SLNIELPIGYYGNAFVTPVVVSKAGLLCSNPVTYAVELIKKVKDHINEE
YIKSVIDLTVIKGRPELTKSWNFLVSDNRYIGFDEFDFGWGNPIFGGIS
KATSFISFGVSVKNDKGEKGVLIAISLPPLAMKKLQDIYNMTERVIIPR
I

In some embodiments, the enzyme having alcohol-O-acyltransferase activity is MpAAT1 (also referred to as MdAAT1) from Malus domestica. The Malus domestica AAT is provided by the amino acid sequence provided by SEQ ID NO: 4, which corresponds UniProtKB Accession No. NP_001315675.1.

(SEQ ID NO: 4)
MMSFSVLQVKRLQPELITPAKSTPQETKFLSDIDDQESLRVQIPIIMCY
KDNPSLNKNRNPVKAIREALSRALVYYYPLAGRLREGPNRKLVVDCNGE
GILFVEASADVTLEQLGDKILPPCPLLEEFLYNFPGSDGIIDCPLLLIQ
VTCLTCGGFILALRLNHTMCDAAGLLLFLTAIAEMARGAHAPSILPVWE
RELLFARDPPRITCAHHEYEDVIGHSDGSYASSNQSNMVQRSFYFGAKE
MRVLRKQIPPHLISTCSTFDLITACLWKCRTLALNINPKEAVRVSCIVN
ARGKHNNVRLPLGYYGNAFAFPAAISKAEPLCKNPLGYALELVKKAKAT
MNEEYLRSVADLLVLRGRPQYSSTGSYLIVSDNTRVGFGDVNFGWGQPV
FAGPVKALDLISFYVQHKNNTEDGILVPMCLPSSAMERFQQELERITQE
PKEDICNNLRSTSQ

In some embodiments, the enzyme having alcohol-O-acyltransferase activity is MpAAT1 and comprises one or more mutations relative to a wild-type amino acid sequence (i.e., SEQ ID NO: 4). The amino acids corresponding to positions 62 and 385 of SEQ ID NO: 4 (MpAAT1), valine at position 62 and asparagine at position 385, are indicated in boldface and underlined in SEQ ID NO: 4 above. In some embodiments, enzyme having alcohol-O-acyltransferase activity is MpAAT1 which has been mutated to substitute a valine at position 62 with an alanine and an asparagine at position 385 with an aspartic acid, as shown in SEQ ID NO: 25.

(SEQ ID NO: 25)
MMSFSVLQVKRLQPELITPAKSTPQETKFLSDIDDQESLRVQIPIIMCY
KDNPSLNKNRNPAKAIREALSRALVYYYPLAGRLREGPNRKLVVDCNGE
GILFVEASADVTLEQLGDKILPPCPLLEEFLYNFPGSDGIIDCPLLLIQ
VTCLTCGGFILALRLNHTMCDAAGLLLFLTAIAEMARGAHAPSILPVWE
RELLFARDPPRITCAHHEYEDVIGHSDGSYASSNQSNMVQRSFYFGAKE
MRVLRKQIPPHLISTCSTFDLITACLWKCRTLALNINPKEAVRVSCIVN
ARGKHNNVRLPLGYYGNAFAFPAAISKAEPLCKNPLGYALELVKKAKAT
MNEEYLRSVADLLVLRGRPQYSSTGSYLIVSDNTRVGFGDVDFGWGQPV
FAGPVKALDLISFYVQHKNNTEDGILVPMCLPSSAMERFQQELERITQE
PKEDICNNLRSTSQ

In some embodiments, the enzyme having alcohol-O-acyltransferase activity is CmAAT1 from Cucumis melo. The Cucumis melo AAT is provided by the amino acid sequence set forth by SEQ ID NO: 5, which corresponds to UniProtKB Accession No. XP_008462821.1.

(SEQ ID NO: 5)
METMQTIDFSFHVRKCQPELIAPANPTPYEFKQLSDVDDQQSLRLQLPF
VNIYPHNPSLEGRDPVKVIKEAIGKALVFYYPLAGRLREGPGRKLFVEC
TGEGILFIEADADVSLEEFWDTLPYSLSSMQNNIIHNALNSDEVLNSPL
LLIQVTRLKCGGFIFGLCFNHTMADGFGIVQFMKATAEIARGAFAPSIL
PVWQRALLTARDPPRITFRHYEYDQVVDMKSGLIPVNSKIDQLFFFSQL
QISTLRQTLPAHLHDCPSFEVLTAYVWRLRTIALQFKPEEEVRFLCVMN
LRSKIDIPLGYYGNAVVVPAVITTAAKLCGNPLGYAVDLIRKAKAKATM
EYIKSTVDLMVIKGRPYFTVVGSFMMSDLTRIGVENVDFGWGKAIFGGP
TTTGARITRGLVSFCVPFMNRNGEKGTALSLCLPPPAMERFRANVHASL
QVKQVVDAVDSHMQTIQSASK

In some embodiments, the heterologous gene encodes an enzyme with alcohol-O-acyltransferase activity such that a cell that expresses the enzyme is capable of increased production of γ-decalactone as compared to a cell that does not express the heterologous gene. In some embodiments, the heterologous gene encodes an enzyme with alcohol-O-acyltransferase activity such that a cell that expresses the enzyme is capable of producing increased levels of γ-decalactone as compared to a cell that expresses an enzyme with wild-type alcohol-O-acyltransferase activity. In some embodiments, the heterologous gene encodes an enzyme with alcohol-O-acyltransferase activity such that a cell that expresses the enzyme is capable of producing reduced levels of ethyl acetate as compared to a cell that does not express the heterologous gene.

In some embodiments, the enzyme with alcohol-O-acyltransferase activity has an amino acid sequence with at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to the sequence as set forth in any one of SEQ ID NOs: 1-5 or 25.

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms are incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% identity) and all increments thereof (e.g., tenths of a percent (i.e., 0.1%), hundredths of a percent (i.e., 0.01%), etc.).

In some embodiments, the enzyme with alcohol-O-acyltransferase activity comprises the amino acid sequence as set forth in any one of SEQ ID NOs: 1-5 or 25. In some embodiments, the enzyme with alcohol-O-acyltransferase activity consists of the amino acid sequence as set forth in any one of SEQ ID NOs: 1-5 or 25. In some embodiments, the enzyme with alcohol-O-acyltransferase activity comprises the amino acid sequence as set forth in SEQ ID NO: 1. In some embodiments, the enzyme with alcohol-O-acyltransferase activity consists of the amino acid sequence as set forth in SEQ ID NO: 1.

In some embodiments, the gene encoding the enzyme with alcohol-O-acyltransferase activity comprises a nucleic acid sequence which encodes an enzyme comprising an amino acid sequence with at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9%) sequence identity to the sequence as set forth in any one of SEQ ID NOs: 1-5 or 25. In some embodiments, the gene encoding the enzyme with alcohol-O-acyltransferase activity comprises a nucleic acid sequence which encodes an enzyme consisting of an amino acid sequence as set forth in any one of SEQ ID NOs: 1-5 or 25.

Identification of additional enzymes having alcohol-O-acyltransferase activity or predicted to have alcohol-O-acyltransferase activity may be performed, for example based on similarity or homology with one or more domains of an alcohol-O-acyltransferase, such as the alcohol-O-acyltransferases provided by any one of SEQ ID NOs: 1-5 or 25. In some embodiments, an enzyme for use in the modified cells and methods described herein may be identified based on similarity or homology with an active domain, such as a catalytic domain, such as a catalytic domain associated with alcohol-O-acyltransferase activity. In some embodiments, an enzyme for use in the modified cells and methods described herein may have a relatively high level of sequence identity with a reference alcohol-O-acyltransferase, e.g., a wild-type alcohol-O-acyltransferase, such as any one of SEQ ID NOs: 1-5 or 25, in the region of the catalytic domain but a relatively low level of sequence identity to the reference alcohol-O-acyltransferase based on analysis of a larger portion of the enzyme or across the full length of the enzyme. In some embodiments, the enzyme for use in the modified cells and methods described herein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity in the region of the catalytic domain of the enzyme relative to a reference alcohol-O-acyltransferase (e.g., SEQ ID NOs: 1-5 or 25).

In some embodiments, an enzyme for use in the modified cells and methods described herein has a relatively high level of sequence identity in the region of the catalytic domain of the enzyme relative to a reference alcohol-O-acyltransferase (e.g., SEQ ID NOs: 1-5 or 25) and a relatively low level of sequence identity to the reference alcohol-O-acyltransferase based on analysis of a larger portion of the enzyme or across the full length of the enzyme. In some embodiments, the enzymes for use in the modified cells and methods described herein have at least 10%, at least 15%, at least 20%, at least 25%, least 30% at least 35%, at least 40%, at least 45%, at least 50%, at least 55%, at least 60%, at least 65%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, or at least 99.9% sequence identity based on a portion of the enzyme or across the full length of the enzyme relative to a reference alcohol-O-acyltransferase (e.g., SEQ ID NOs: 1-5 or 25).

In some embodiments, the gene encoding the enzyme with alcohol-O-acyltransferase activity further comprises a localization signal. The term “localization signal,” as used herein, refers to a short peptide sequence (typically less than 70 amino acids) present at the terminus (N-terminus or C-terminus) of a newly synthesized protein that facilitates the transport or trafficking of the newly synthesized protein to a target region of the cell (e.g., the cell membrane or an organelle).

In some embodiments, the localization signal is a peroxisome targeting signal. The term “peroxisome targeting signal,” as used herein, refers to a peptide sequence at the N-terminus of a newly synthesized protein that facilitates the transport or trafficking of the newly synthesized protein to the peroxisome. Peroxisomes and mitochondria are the primary sites of beta-oxidation in eukaryotic cells, which beta-oxidation is involved in the production of γ-decalactone, as described herein (FIG. 2). Without wishing to be bound by any particular theory, it is thought that localizing an enzyme having AAT activity to the peroxisome may increase beta-oxidation and thus production of γ-decalactone. In some embodiments, a peroxisome targeting signal is fused to the C-terminus of the enzyme having AAT activity, such as any one of SEQ ID NOs: 1-5 or 25.

In some embodiments, the peroxisome targeting signal comprises the amino acid sequence SKL (SEQ ID NO: 17). In some embodiments, the peroxisome targeting signal comprises the amino acid sequence GSLGRGRRSKL (SEQ ID NO: 18).

### General Methods of Genetic Engineering

As will also be evident to one or ordinary skill in the art, the amino acid position number of a selected residue in a fatty acid hydroxylase, a deregulated transcription factor, acyl-CoA desaturase 1, and/or an alcohol-O-acyltransferase enzyme may have a different amino acid position number as compared to another fatty acid hydroxylase, transcription factor, acyl-CoA desaturase 1 enzyme, or alcohol-O-acyltransferase (e.g., a reference enzyme). Generally, one may identify corresponding positions in other fatty acid hydroxylase, deregulated transcription factor, acyl-CoA desaturase 1, and/or alcohol-O-acyltransferase enzymes using methods known in the art, for example by aligning the amino acid sequences of two or more enzymes. Software programs and algorithms for aligning amino acid (or nucleotide) sequences are known in the art and readily available, e.g., Clustal Omega (Sievers et al. 2011).

The fatty acid hydroxylase, transcription factor, acyl-CoA desaturase 1, and/or alcohol-O-acyltransferase enzymes described herein may further contain one or more modifications, for example to specifically alter a feature of the polypeptide unrelated to its desired physiological activity. Alternatively or in addition, the fatty acid hydroxylase, transcription factor, acyl-CoA desaturase 1, and/or alcohol-O-acyltransferase enzymes described herein may contain one or more mutations to modulate expression and/or activity of the enzyme in the cell.

Mutations of a nucleic acid which encodes an fatty acid hydroxylase, transcription factor, acyl-CoA desaturase 1, and/or alcohol-O-acyltransferase preferably preserve the amino acid reading frame of the coding sequence, and preferably do not create regions in the nucleic acid which are likely to hybridize to form secondary structures, such a hairpins or loops, which can be deleterious to expression of the enzyme.

Mutations can be made by selecting an amino acid substitution, or by random mutagenesis of a selected site in a nucleic acid which encodes the polypeptide. As described herein, variant polypeptides can be expressed and tested for one or more activities to determine which mutation provides a variant polypeptide with the desired properties. Further mutations can be made to variants (or to non-variant polypeptides) which are silent as to the amino acid sequence of the polypeptide, but which provide preferred codons for translation in a particular host (referred to as codon optimization). The preferred codons for translation of a nucleic acid in, e.g., S. cerevisiae, are well known to those of ordinary skill in the art. Still other mutations can be made to the noncoding sequences of a gene or cDNA clone to enhance expression of the polypeptide. The activity of a fatty acid hydroxylase, transcription factor, acyl-CoA desaturase 1 (enzyme), and/or an alcohol-O-acyltransferase variant can be tested by cloning the gene encoding the enzyme variant into an expression vector, introducing the vector into an appropriate host cell, expressing the enzyme variant, and testing for a functional capability of the enzyme, as disclosed herein.

The fatty acid hydroxylase, transcription factor, acyl-CoA desaturase 1, and/or alcohol-O-acyltransferase enzymes described herein may contain an amino acid substitution of one or more positions corresponding to a reference fatty acid hydroxylase, transcription factor, acyl-CoA desaturase 1, and/or alcohol-O-acyltransferase, such as a wild-type transcription factor or enzyme. In some embodiments, the fatty acid hydroxylase, transcription factor, acyl-CoA desaturase 1 enzyme, and/or alcohol-O-acyltransferase contains an amino acid substitution at 1, 2, 3, 4, 5, or more positions corresponding to a reference fatty acid hydroxylase, transcription factor, acyl-CoA desaturase 1, and/or alcohol-O-acyltransferase. In some embodiments, the fatty acid hydroxylase, transcription factor, acyl-CoA desaturase 1, and/or alcohol-O-acyltransferase is not a naturally occurring alcohol-O-acyltransferase, fatty acid hydroxylase, and/or acyl-CoA desaturase 1, e.g., is genetically modified.

In some embodiments, the fatty acid hydroxylase, transcription factor, acyl-CoA desaturase 1, and/or alcohol-O-acyltransferase variant may also contain one or more amino acid substitutions that do not substantially affect the activity and/or structure of the fatty acid hydroxylase, transcription factor, acyl-CoA desaturase 1 and/or alcohol-O-acyltransferase enzyme. The skilled artisan will also realize that conservative amino acid substitutions may be made in the enzyme to provide functionally equivalent variants of the foregoing polypeptides, i.e., the variants retain the functional capabilities of the polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Exemplary functionally equivalent variants of polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

As one of ordinary skill in the art would be aware, homologous genes encoding a fatty acid hydroxylase, deregulated transcription factor, acyl-CoA desaturase 1, and/or alcohol-O-acyltransferase activity could be obtained from other species and could be identified by homology searches, for example through a protein BLAST search, available at the National Center for Biotechnology Information (NCBI) internet site (ncbi.nlm.nih.gov). By aligning the amino acid sequence of an enzyme with one or more reference enzymes and/or by comparing the secondary or tertiary structure of a similar or homologous enzyme with one or more reference enzymes, one can determine corresponding amino acid residues in similar or homologous enzymes and can determine amino acid residues for mutation in the similar or homologous enzyme. Similarly, by aligning the amino acid sequence of transcription factor with one or more reference transcription factors and/or by comparing the secondary or tertiary structure of a similar or homologous transcription factors with one or more reference transcription factors, one can determine corresponding amino acid residues in similar or homologous transcription factors and can determine amino acid residues for mutation in the similar or homologous transcription factors.

Genes associated with the disclosure can be obtained (e.g., by PCR amplification) from DNA from any source of DNA which contains the given gene. In some embodiments, genes associated with the invention are synthetic, e.g., produced by chemical synthesis in vitro. Any means of obtaining a gene encoding the enzymes described herein are compatible with the modified cells and methods described herein.

The disclosure provided herein involves recombinant expression of genes encoding a fatty acid hydroxylase, deregulated transcription factor, acyl-CoA desaturase 1, and/or alcohol-O-acyltransferase, functional modifications and variants of the foregoing, as well as uses relating thereto. Homologs and alleles of the nucleic acids associated with the invention can be identified by conventional techniques. Also encompassed by the invention are nucleic acids that hybridize under stringent conditions to the nucleic acids described herein. The term “stringent conditions” as used herein refers to parameters with which the art is familiar. Nucleic acid hybridization parameters may be found in references which compile such methods, e.g., Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code. The invention also embraces codon optimization to suit optimal codon usage of a host cell.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as enzymatic activity. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two, or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

In one aspect of the present disclosure, one or more of the genes associated with the invention is expressed in a recombinant expression vector. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., 0-galactosidase, luciferase, or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined or operably linked when they are covalently linked in such a way as to place the expression or transcription of the coding sequence under the influence or control of the regulatory sequences. If it is desired that the coding sequences be translated into a functional protein, two DNA sequences are said to be operably joined or operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the present disclosure is expressed in a cell, a variety of transcription control sequences (e.g., promoter/enhancer sequences) can be used to direct its expression. In some embodiments, each of the genes is operably linked to a promoter (e.g., each gene linked to a separate promoter). The promoter can be a native promoter, i.e., the promoter of the gene in its endogenous context, which provides normal regulation of expression of the gene. In some embodiments, the promoter can be constitutive, i.e., the promoter is unregulated allowing for continual transcription of its associated gene (e.g., fatty acid hydroxylase, deregulated transcription factor, acyl-CoA desaturase 1, alcohol-O-acyltransferase). A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) may be placed under operable control of transcriptional elements to permit the expression of the heterologous DNA in the host cell. As one of ordinary skill in the art would appreciate, any of the enzymes described herein can also be expressed in other yeast cells, including yeast strains used for producing wine, mead, sake, cider, etc.

A nucleic acid molecule that encodes the enzyme of the present disclosure can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

The incorporation of genes can be accomplished either by incorporation of the nucleic acid encoding the enzyme(s) into the genome of the yeast cell, or by transient or stable maintenance of the new nucleic acid encoding the enzyme(s) as an episomal element. In eukaryotic cells, a permanent, inheritable genetic change is generally achieved by introduction of the DNA into the genome of the cell.

The heterologous gene may also include various transcriptional elements required for expression of the encoded gene product (e.g., fatty acid hydroxylase, transcription factor, acyl-CoA desaturase 1, alcohol-O-acyltransferase). For example, in some embodiments, the gene may include a promoter. In some embodiments, the promoter may be operably joined to the gene. In some embodiments, the cell is an inducible promoter. In some embodiments, the promoter is active during a particular stage of a fermentation process. For example, in some embodiments, peak expression from the promoter is during an early stage of the fermentation process, e.g., before >50% of the fermentable sugars have been consumed. In some embodiments, peak expression from the promoter is during a late stage of the fermentation process e.g., after 50% of the fermentable sugars have been consumed.

Conditions in the medium change during the course of the fermentation process, for example the availability of nutrients and oxygen tend to decrease over time during fermentation as sugar source and oxygen become depleted. Additionally, the presence of other factors, such as products produced by metabolism of the cells, may increase. In some embodiments, the promoter is regulated by one or more conditions in the fermentation process, such as presence or absence of one or more factors. In some embodiments, the promoter is regulated by hypoxic conditions. Examples of promoters of hypoxia activated genes are known in the art. See, e.g., Zitomer et al. Kidney Int. (1997) 51(2): 507-13; Gonzalez Siso et al. Biotechnol. Letters (2012) 34: 2161-2173.

In some embodiments, the promoter is a constitutive promoter. Examples of constitutive promoters for use in yeast cells are known in the art and evident to one of ordinary skill in the art. In some embodiments, the promoter is a yeast promoter, e.g., a native promoter from the yeast cell in which the heterologous gene or the exogenous gene is expressed.

Non-limiting examples of promoters for use in the genetically modified cells and methods described herein include, the HEM13 promoter (pHEM13), SPG1 promoter (pSPG1), PRB1 promoter (pPRB1), QCR10 (pQCR10), PGK1 promoter (pPGK1), OLE1 promoter (pOLE1), ERG25 promoter (pERG25), the HHF2 promoter (pHHF2), the TDH1 promoter (pTDH1), the TDH2 promoter (pTDH2), the TDH3 promoter (pTDH3), the ENO2 promoter (pENO2), the HSP26 promoter (pHSP26), or the RPL18b promoter (pRPL18b).

An exemplary HEM13 promoter is pHEM13 from S. cerevisiae, which is provided by the nucleotide sequence set forth as SEQ ID NO: 8.

(SEQ ID NO: 8)
TAATGTAGAAGGTTGAGAACAACCGGATCTTGCGGTCATTTTTCTTTTC
GAGGAAAGTGCAAGTCTGCCACTTTCCAGAAGGCATAGCCTTGCCCTTT
TGTTGATATTTCTCCCCACCGTAATTGTTGCATTCGCGATCTTTTCAAC
AATACATTTTATCATCAAGCCCGCAAATCCTCTGGAGTTTGTCCTCTCG
TTCACTGTTGGGAAAAACAATACGCCTAATTCGTGATTAAGATTCTTCA
AACCATTTCCTGCGGAGTTTTTACTGTGTGTTGAACGGTTCACAGCGTA
AAAAAAAGTTACTATAGGCACGGTATTTTAATTTCAATTGTTTAGAAAG
TGCCTTCACACCATTAGCCCCTGGGATTACCGTCATAGGCACTTTCTGC
TGAGCTCCTGCGAGATTTCTGCGCTGAAAGAGTAAAAGAAATCTTTCAC
AGCGGCTCCGCGGGCCCTTCTACTTTTAAACGAGTCGCAGGAACAGAAG
CCAAATTTCAAAGAACGCTACGCTTTCGCCTTTTCTGGTTCTCCCACCA
ATAACGCTCCAGCTTGAACAAAGCATAAGACTGCAACCAAAGCGCTGAC
GGACGATCCGAAGATAAAGCTTGCTTTGCCCATTGTTCTCGTTTCGAAA
GGCTATATAAGGACACGGATTTTCCtTTTTTTTTTCCACCTATTGTCTT
TCTTTGTTAAGCTTTTATTCTCCGGGTTTTTTTTTTTTGAGCATATCAA
AAGCTTTCTTTTCGCAAATCAAACATAGCAAACCGAACTCTTCGAACAC
AATTAAATACACATAAA

An exemplary SPG1 promoter is pSPG1 from S. cerevisiae, which is provided by the nucleotide sequence set forth as SEQ ID NO: 9.

(SEQ ID NO: 9)
ATGAAGTTCACTTCACATCCAATGAGAAAAACAAAATCCGCAGGGCTAT
CACCCAGAACATCCTCCACTTCATCTTCTTCAGGACAGAGAAAAGCGCA
TCACCACCACCATCACCACAACCACGTTTCAAGGACGAAAACTACCGAA
AGCACCAAATCAGGCAACAGCAAAAAGGACAGTTCCTCATCCTCAACAA
ACGACCATCAATTTAAAAGGTCTGAAAAGAAGAAAAAAAGTAAATTTGG
CTCGATCTTCAAAAAAGTTTTCGGATGAACCGGATTAATACAAGTAAAA
TCAGCAAAGATATAGAAGACAAAATAAGCGTGAAAACAATCATAAACCA
CTCACAACGGGGGTTTTCAGCTGTTACTCCTCCATACATACATTTTGAT
AAAGATATAATGTTATATTTCTTTTCGTAATTTTGTTTTACTTCGGTTT
GCTCTATAGATTTCATCAGCCGCACCGAAAAGGGAGATCAATAAGGTAC
CCTTTAAAAGGGATAAGAAGCCTAACATCACCCCAATAAATGGAGTAAT
GGCCAGCATTGGATGAAGAGAAGAATTACGGGATACTGGGATAACACTG
TTAAAAATGCTTCGCGACGTGAGGGTCTTATATAAATTGAACTGCCAAA
TCTCTTTCACATTATCCAGGATAGTTTGGAATGTGTGTTACTGAAAGAT
CAGAATCAATAAATACAATCAATACAAATATTTAGCGCATAAAATTCAA
ACAAAGTTTACTGAA

An exemplary PRB1 promoter is pPRB1 from S. cerevisiae, which is provided by the nucleotide sequence set forth as SEQ ID NO: 10.

(SEQ ID NO: 10)
CGAGAAACAGGGGGGGAGAAAAGGGGAAAAGAGAAGGAAAGAAAGACTC
ATCTATCGCAGATAAGACAATCAACCCTCATGGCGCCTCCAACCACCAT
CCGCACTAGGGACCAAGCGCTCGCACCGTTAGCAACGCTTGACTCACAA
ACCAACTGCCGGCTGAAAGAGCTTGTGCAATGGGAGTGCCAATTCAAAG
GAGCCGAATACGTCTGTTCGCCTTTTAAGAGGCTTTTTGAACACTGCAT
TGCACCCGACAAATCAGCCACTAACTACGAGGTCACGGATACATATACC
AATAGTTAAAAAATTACATATACTCTATATAGCACAGTAGTGTGATAAA
TAAAAAATTTTGCCAAGACTTTTTTAAACTGCACCCGACAGATCAGGTC
TGTGCCTACTATGCACTTATGCCCGGGGTCCCGGGAGGAGAAAAAACGA
GGGCTGGGAAATGTCCGTGGACTTAAAACGCTCCGGGTTAGCAGAGTAG
CAGGGCTTTCGGCTTTGGAAATTTAGGTGACTTGTTGAAAAAGCAAAAT
TTGGGCTCAGTAATGCCACaGCAGTGGCTTATCACGCCAGGACTGCGGG
AGTGGCGGGGGCAAACACACCCGCGATAAAGAGCGCGATGAATATAAAA
GGGGGCCAATGTTACGTCCCGTTATATTGGAGTTCTTCCCATACAAACT
TAAGAGTCCAATTAGCTTCATCGCCAATAAAAAAACAAACTAAACCTAA
TTCTAACAAGCAAAG

An exemplary QCR10 promoter is pQCR10 from S. cerevisiae, which is provided by the nucleotide sequence set forth as SEQ ID NO: 11.

(SEQ ID NO: 11)
GAGAGCTGGCCAAAAAGAGGGCCGAAGACGGCGTTGAATTTCATTCAAA
ACTATTTAGAAGGGCAGAGCCAGGTGAGGATTTAGATTATTATATTTAC
AAGCACATCCCTGAAGGGACCGACAAGCATGAAGAACAGATCAGGAGCA
TTTTGGAAACTGCCCCGATTTTACCAGGACAGGCATTCACTGAAAAATT
TTCTATTCCGGCTTATAAAAAGCATGGAATCCAAAAGAATTAGGCTTCT
CATTCTATTTTAATTATACTAGTACGATTTCTCACTCTGTAATTTAATA
TCAGTGTAATATGCACCTAGTTATGGGTAGTTTTTGCTAACGTTACGAG
CCGCGAAACTGTCCTCAATCTTCACCACTACCTCTAATGACTGAAGAAT
GCTATGCGATATAACGCTGCCGCACTTTGAATATATACTTATATTTACA
TAGTTTTCAAGTGCGTATTACTATTGCAAAGTAGTATTTTGTCACGTGA
TTTTGATCCAATTAAAACTAAATATGGTTCAACCCGTTGTTTCCGCATC
AAAAAACCATACCATTTATCAAGGGGACGGGATATATCACATAACAGTT
TGAATGCATAATTTGTTATAGATATCTTCTGGAATAATCTTCACAGCAA
AAGCGCAAGTCGAATAATATATCGATAAATACAATCCATAAGACTTAAA
ACTAACCTCA

An exemplary TDH1 promoter is pTDH1 from S. cerevisiae, which is provided by the nucleotide sequence set forth in SEQ ID NO: 12.

(SEQ ID NO: 12)
GCCCGCTTCTGAAAACTACAGTTGACTTGTATGCTAAAGGGCCAGACTA
ATGGGAGGAGAAAAAGAAACGAATGTATATGCTCATTTACACTCTATAT
CACCATATGGAGGATAAGTTGGGCTGAGCTTCTGATCCAATTTATTCTA
TCCATTAGTTGCTGATATGTCCCACCAGCCAACACTTGATAGTATCTAC
TCGCCATTCACTTCCAGCAGCGCCAGTAGGGTTGTTGAGCTTAGTAAAA
ATGTGCGCACCACAAGCCTACATGACTCCACGTCACATGAAACCACACC
GTGGGGCCTTGTTGCGCTAGGAATAGGATATGCGACGAAGACGCTTCTG
CTTAGTAACCACACCACATTTTCAGGGGGTCGATCTGCTTGCTTCCTTT
ACTGTCACGAGCGGCCCATAATCGCGCTTTTTTTTTAAAAGGCGCGAGA
CAGCAAACAGGAAGCTCGGGTTTCAACCTTCGGAGTGGTCGCAGATCTG
GAGACTGGATCTTTACAATACAGTAAGGCAAGCCACCATCTGCTTCTTA
GGTGCATGCGACGGTATCCACGTGCAGAACAACATAGTCTGAAGAAGGG
GGGGAGGAGCATGTTCATTCTCTGTAGCAGTAAGAGCTTGGTGATAATG
ACCAAAACTGGAGTCTCGAAATCATATAAATAGACAATATATTTTCACA
CAATGAGATTTGTAGTACAGTTCTATTCTCTCTCTTGCATAAATAAGAA
ATTCATCAAGAACTTGGTTTGATATTTCACCAACACACACAAAAAACAG
TACTTCACTAAATTTACACACAAAACAAA

An exemplary TDH2 promoter is pTDH2 from S. cerevisiae, which is provided by the nucleotide sequence set forth in SEQ ID NO: 13.

(SEQ ID NO: 13)
CTAAATACTTCTGTGTTTTCATTAATTTATAAATTGTACTCTTTTAAGA
CATGGAAAGTACCAACATCGGTTGAAACAGTTTTTCATTTACTTATGGT
TTATTGGTTTTTCCAGTGAATGATTATTTGTCGTTACCCTTTCGTAAAA
GTTCAAACACGTTTTTAAGTATTGTTTAGTTGCTCTTTCGACATATATG
ATTATCCCTGCGCGGCTAAAGTTAAGGATGCAAAAAACATAAGACAACT
GAAGTTAATTTACGTCAATTAAGTTTTCCAGGGTAATGATGTTTTGGGC
TTCCACTAATTCAATAAGTATGTCATGAAATACGTTGTGAAGAGCATCC
AGAAATAATGAAAAGAAACAACGAAACTGGGTCGGCCTGTTGTTTCTTT
TCTTTACCACGTGATCTGCGGCATTTACAGGAAGTCGCGCGTTTTGCGC
AGTTGTTGCAACGCAGCTACGGCTAACAAAGCCTAGTGGAACTCGACTG
ATGTGTTAGGGCCTAAAACTGGTGGTGACAGCTGAAGTGAACTATTCAA
TCCAATCATGTCATGGCTGTCACAAAGACCTTGCGGACCGCACGTACGA
ACACATACGTATGCTAATATGTGTTTTGATAGTACCCAGTGATCGCAGA
CCTGCAATTTTTTTGTAGGTTTGGAAGAATATATAAAGGTTGCACTCAT
TCAAGATAGTTTTTTTCTTGTGTGTCTATTCATTTTATTATTGTTTGTT
TAAATGTTAAAAAAACCAAGAACTTAGTTTCAAATTAAATTCATCACAC
AAACAAACAAAACAAA

An exemplary TDH3 promoter is pTDH3 from S. cerevisiae, which is provided by the nucleotide sequence set forth in SEQ ID NO: 14.

(SEQ ID NO: 14)
CAGTTCGAGTTTATCATTATCAATACTGCCATTTCAAAGAATACGTAAA
TAATTAATAGTAGTGATTTTCCTAACTTTATTTAGTCAAAAAATTAGCC
TTTTAATTCTGCTGTAACCCGTACATGCCCAAAATAGGGGGCGGGTTAC
ACAGAATATATAACATCGTAGGTGTCTGGGTGAACAGTTTATTCCTGGC
ATCCACTAAATATAATGGAGCCCGCTTTTTAAGCTGGCATCCAGAAAAA
AAAAGAATCCCAGCACCAAAATATTGTTTTCTTCACCAACCATCAGTTC
ATAGGTCCATTCTCTTAGCGCAACTACAGAGAACAGGGGCACAAACAGG
CAAAAAACGGGCACAACCTCAATGGAGTGATGCAACCTGCCTGGAGTAA
ATGATGACACAAGGCAATTGACCCACGCATGTATCTATCTCATTTTCTT
ACACCTTCTATTACCTTCTGCTCTCTCTGATTTGGAAAAAGCTGAAAAA
AAAGGTTGAAACCAGTTCCCTGAAATTATTCCCCTACTTGACTAATAAG
TATATAAAGACGGTAGGTATTGATTGTAATTCTGTAAATCTATTTCTTA
AACTTCTTAAATTCTACTTTTATAGTTAGTCTTTTTTTTAGTTTTAAAA
CACCAAGAACTTAGTTTCGAATAAACACACATAAACAAACAAA

An exemplary ENO2 promoter is pENO2 from S. cerevisiae, which is provided by the nucleotide sequence set forth in SEQ ID NO: 15.

(SEQ ID NO: 15)
ATTGAATACATTAGCAACGCGTCCAGCATTTTTCGGAAGTGTCTCATAA
ACTTTACTCAAGAGTTAAGTACTGAAAAATTCGACTTTTATGATAGTTC
AAGTGTCGACGCTGCGGGTATAGAAAGGGTTCTTTACTCTATAGTGCCT
CCTCGCTCAGCATCTGCTTCTTCCCAAAGATGAACGCGGCGTTATGTCA
CTAACGACGTGCACCAACTTGCGGAAAGTGGAATCCCGTTCCAAAACTG
GCATCCACTAATTGATACATCTACACACCGCACGCCTTTTTTCTGAAGC
CCACTTTCGTGGACTTTGCCATATGCAAAATTCATGAAGTGTGATACCA
AGTCAGCATACACCTCACTAGGGTAGTTTCTTTGGTTGTATTGATCATT
TGGTTCATCGTGGTTCATTAATTTTTTTTCTCCATTGCTTTCTGGCTTT
GATCTTACTATCATTTGGATTTTTGTCGAAGGTTGTAGAATTGTATGTG
ACAAGTGGCACCAAGCATATATAAAAAAAAAAGCATTATCTTCCTACCA
GAGTTGATTGTTAAAAACGTATTTATAGCAAACGCAATTGTAATTAATT
CTTATTTTGTATCTTTTCTTCCCTTGTCTCAATCTTTTATTTTTATTTT
ATTCTTCTTTTCTTAGTTTCTTTCATAACACCAAGCAACTAATACTATA
ACATACAATAATA

An exemplary HSP26 promoter is pHSP26 from S. cerevisiae, which is provided by the nucleotide sequence set forth in SEQ ID NO: 16.

(SEQ ID NO: 16)
CAATATTCTGCGCACATCAATCATTTTCTTACTACATACACTAACATTA
CTCCTAGTTTAATTTAATTGAATTTTTAACTTTCTTTTCTTTTCATTTG
GCAATTTGGCTCCTTGAAAACAAGACTATGGGTCTgTCTCATAAGCCTC
AGGGGGGGACCCCAAAAAAATAACGCGGCCATCTTGCATGCACCGTTGA
ACCTGTAGCTTACAGTAAGCCACAATTCTCTTACCTTCTTGGCAATGTG
GCACAAAATAATCTGGTTATGTGTCTTCATTTGGTAATCACTGGGATGT
TACTGGGGCAGCAGCAACTCCGTGTGTACCCCTAACTCCGTGTGTACCC
CTAAAGAACCTTGCCTGTCAAGGTGCATTGTTGGATCGGAATAGTAACC
GTCTTTACATGAACATCCACAACCAACGAAAGTGCTTTTTCAAGCATTG
CTTGATTTCTAGAAAGATCGATGGTTATTCCCTCCCCCTTATGCGTCCA
AAAATATAGGGTGCTCGTAACAGTAAGGTATTCGCACTTAGCGTGCTCG
CAACACAAAATTAAGTAATATGCGAGTTTTAGATGTCCTTGCGGATCTA
TGCACGTTCTTGAGTGGTATTTCATAACAACGGTTCTTTTTCACCCTTA
TTCCTAAACATATAAATAGGACCTCCATTAGTTAGAGATCTGTTTTTAA
TCCATTCACCTTTCATTCTACTCTCTTATACTAATAAAACCACCGATAA
AGATATATCAGATCTCTATTAAAACAGGTATCCAAAAAAGCAAACAAAC
AAACTAAACAAATTAA

An exemplary RPL18b promoter is pRPL18b from S. cerevisiae, which is provided by the nucleotide sequence set forth in SEQ ID NO: 19.

(SEQ ID NO: 19)
AAGAGGATGTCCAATATTTTTTTTAAGGAATAAGGATACTTCAAGACTA
GATTCCCCCCTGCATTCCCATCAGAACCGTAAACCTTGGCGCTTTCCTT
GGGAAGTATTCAAGAAGTGCCTTGTCCGGTTTCTGTGGCTCACAAACCA
GCGCGCCCGATATGGCTTTCTTTTCACTTATGAATGTACCAGTACGGGA
CAATTAGAACGCTCCTGTAACAATCTCTTTGCAAATGTGGGGTTACATT
CTAACCATGTCACACTGCTGACGAAATTCAAAGTAAAAAAAAATGGGAC
CACGTCTTGAGAACGATAGATTTTCTTTATTTTACATTGAACAGTCGTT
GTCTCAGCGCGCTTTATGTTTTCATTCATACTTCATATTATAAAATAAC
AAAAGAAGAATTTCATATTCACGCCCAAGAAATCAGGCTGCTTTCCAAA
TGCAATTGACACTTCATTAGCCATCACACAAAACTCTTTCTTGCTGGAG
CTTCTTTTAAAAAAGACCTCAGTACACCAAACACGTTACCCGACCTCGT
TATTTTACGACAACTATGATAAAATTCTGAAGAAAAAATAAAAAAATTT
TCATACTTCTTGCTTTTATTTAAACCATTGAATGATTTCTTTTGAACAA
AACTACCTGTTTCACCAAAGGAAATAGAAAGAAAAAATCAATTAGAAGA
AAACAAAAAACAAA

### Genetically Modified Yeast Cells

Aspects of the present disclosure relates to genetically modified yeast cells (modified cells) and use of such modified cells in methods of producing a fermented product (e.g., a fermented beverage) and methods of producing ethanol. The genetically modified yeast cells described herein are genetically modified with a heterologous gene encoding an enzyme with a heterologous gene encoding an enzyme with fatty acid hydroxylase activity, a gene encoding a deregulated transcription factor, and/or a gene encoding an enzyme with acyl-CoA desaturase 1 activity. In some embodiments, the cells described herein are genetically modified with a heterologous gene encoding an enzyme with alcohol-O-acyltransferase activity.

The terms “genetically modified cell,” “genetically modified yeast cell,” and “modified cell,” as may be used interchangeably herein, to refer to a eukaryotic cell (e.g., a yeast cell), which has been, or may be presently, modified by the introduction of a heterologous gene. The terms (e.g., modified cell) include the progeny of the original cell which has been genetically modified by the introduction of a heterologous gene. It shall be understood by the skilled artisan that the progeny of a single cell may not necessarily be completely identical in morphology or in genomic or total nucleic acid complement as the original parent, due to mutation (i.e., natural, accidental, or deliberate alteration of the nucleic acids of the modified cell).

Yeast cells for use in the methods described herein are preferably capable of fermenting a sugar source (e.g., a fermentable sugar) and producing ethanol (ethyl alcohol) and carbon dioxide. In some embodiments, the yeast cell is of the genus Saccharomyces. The Saccharomyces genus includes nearly 500 distinct of species, many of which are used in food production. One example species is Saccharomyces cerevisiae (S. cerevisiae), which is commonly referred to as “brewer's yeast” or “baker's yeast,” and is used in the production of wine, bread, beer, among other products. Other members of the Saccharomyces genus include, without limitation, the wild yeast Saccharomyces paradoxus, which is a close relative to S. cerevisiae; Saccharomyces bayanus, Saccharomyces pastorianus, Saccharomyces carlsbergensis, Saccharomyces uvarum, Saccharomyces cerevisiae var boulardii, Saccharomyces eubayanus. In some embodiments, the yeast is Saccharomyces cerevisiae (S. cerevisiae).

Saccharomyces species may be haploid (i.e., having a single set of chromosomes), diploid (i.e., having a paired set of chromosomes), or polyploid (i.e., carrying or containing more than two homologous sets of chromosomes). Saccharomyces species used, for example for beer brewing, are typically classified into two groups: ale strains (e.g., S. cerevisiae), which are top fermenting, and lager strains (e.g., S. pastorianus, S. carlsbergensis, S. uvarum), which are bottom fermenting. These characterizations reflect their separation characteristics in open square fermentors, as well as often other characteristics such as preferred fermentation temperatures and alcohol concentrations achieved.

Although beer brewing and wine producing has traditionally focused on use of S. cerevisiae strains, other yeast species and genera have been appreciated in production of fermented beverages. In some embodiments, the yeast cell belongs to a non-Saccharomyces genus. See, e.g., Crauwels et al. Brewing Science (2015) 68: 110-121; Esteves et al. Microorganisms (2019) 7(11): 478. In some embodiments, the yeast cell is of the genus Kloeckera, Candida, Starmerella, Hanseniaspora, Kluyveromyces/Lachance, Metschnikowia, Saccharomycodes, Zygosaccharomyces, Dekkera (also referred to as Brettanomyces), Wickerhamomyces, or Torulaspora. Examples of non-Saccharomyces yeast include, without limitation, Hanseniaspora uvarum, Hanseniaspora guillermondii, Hanseniaspora vinae, Metschnikowia pulcherrima, Kluyveromyces Lachancea thermotolerans, Starmerella bacillaris (previously referred to as Candida stellata/Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, and Torulaspora delbrueckii.

In some embodiments, the methods described herein involve use of more than one genetically modified yeast. For example, in some embodiments, the methods may involve use of more than one genetically modified yeast belonging to the genus Saccharomyces. In some embodiments, the methods may involve use of more than one genetically modified yeast belonging to a non-Saccharomyces genus. In some embodiments, the methods may involve use of more than one genetically modified yeast belonging to the genus Saccharomyces and one genetically modified yeast belonging to a non-Saccharomyces genus. Alternatively, or in addition, the any of the methods described herein may involve use of one or more genetically modified yeast and one or more non-genetically modified (wildtype) yeast.

In some embodiments, the yeast is a hybrid strain. As will be evident to one of ordinary skill in the art, the term “hybrid strain” of yeast refers to a yeast strain that has resulted from the crossing of two different yeast strains, for example, to achieve one or more desired characteristics. For example, a hybrid strain may result from the crossing of two different yeast strains belonging to the same genus or the same species. In some embodiments, a hybrid strain results from the crossing of a Saccharomyces cerevisiae strain and a Saccharomyces eubayanus strain. See, e.g., Krogerus et al. Microbial Cell Factories (2017) 16: 66.

In some embodiments, the yeast strain is a wild yeast strain, such as a yeast strain that is isolated from a natural source and subsequently propagated. Alternatively, in some embodiments, the yeast strain is a domesticated yeast strain. Domesticated yeast strains have been subjected to human selection and breeding to have desired characteristics.

In some embodiments, the genetically modified yeast cells may be used in symbiotic matrices with other yeast or bacterial strains. Symbiotic matrices of yeast cells and bacterial strains may be used, for example, for the production of fermented beverages, such as kombucha, kefir, and ginger beers. Saccharomyces fragilis, for example, is part of kefir culture and is grown on the lactose contained in whey. Other bacterial strains that may be used in symbiotic matrices with the genetically modified yeast cells include Bifidobacterium animalis subsp. lactis, Bifidobacterium breve, bacteria in the genus Lactobacillus, and bacteria in the genus Pediococcus.

Although many fermented beverages are produced using S. cerevisiae strains, other yeast genera have been appreciated in production of fermented beverages and may be used in symbiotic matrices with the modified yeast cells. In some embodiments, the other yeast cell belongs to a non-Saccharomyces genus. See, e.g., Crauwels et al. Brewing Science (2015) 68: 110-121; Esteves et al. Microorganisms (2019) 7(11): 478. In some embodiments, the other yeast cell is of the genus Kloeckera, Candida, Starmerella, Hanseniaspora, Kluyveromyces/Lachance, Metschnikowia, Saccharomycodes, Zygosaccharomyce, Dekkera (also referred to as Brettanomyces), Wickerhamomyces, or Torulaspora. Examples of non-Saccharomyces yeast include, without limitation, Hanseniaspora uvarum, Hanseniaspora guillermondii, Hanseniaspora vinae, Metschnikowia pulcherrima, Kluyveromyces Lachancea thermotolerans, Starmerella bacillaris (previously referred to as Candida stellata/Candida zemplinina), Saccharomycodes ludwigii, Zygosaccharomyces rouxii, Dekkera bruxellensis, Dekkera anomala, Brettanomyces custersianus, Brettanomyces naardenensis, Brettanomyces nanus, Wickerhamomyces anomalus, and Torulaspora delbrueckii.

Methods of genetically modifying yeast cells are known in the art. In some embodiments, the yeast cell is diploid and one copy of a heterologous gene encoding an enzyme with alcohol-O-acyltransferase activity as described herein is introduced into the yeast genome.

In some embodiments, the yeast cell is diploid and one copy of a gene encoding an enzyme with fatty acid synthase activity as described herein is introduced into both copies of the yeast genome. In some embodiments, the copies of the gene encoding an enzyme with fatty acid hydroxylase activity are identical. In some embodiments, the copies of the gene encoding an enzyme with fatty acid hydroxylase activity are not identical, but the genes encode an identical enzyme having fatty acid hydroxylase activity. In some embodiments, the copies of the gene encoding an enzyme with fatty acid hydroxylase activity are not identical, and the genes encode enzymes having fatty acid synthase activity that are different (e.g., mutants, variants, fragments thereof). In some embodiments, the cell contains a gene encoding an enzyme with fatty acid hydroxylase activity, referred to as an endogenous gene, and also contains a second gene encoding an enzyme with fatty acid hydroxylase activity, which may be the same or different enzyme with fatty acid hydroxylase activity as that encoded by the endogenous gene.

In some embodiments, the yeast cell is diploid and one copy of a gene encoding a transcription factor (e.g., a deregulated transcription factor) as described herein is introduced into both copies of the yeast genome. In some embodiments, the copies of the gene are identical. In some embodiments, the copies of the gene are not identical, but the genes encode an identical transcription factors or transcription factors having identical or substantially similar activity. In some embodiments, the copies of the gene are not identical, and the genes encode transcription factors that are different (e.g., mutants, variants, fragments thereof).

In some embodiments, the yeast cell is diploid and one copy of a heterologous gene encoding an enzyme with acyl-CoA desaturase 1 activity as described herein is introduced into both copies of the yeast genome. In some embodiments, the copies of the heterologous gene are identical. In some embodiments, the copies of the heterologous gene are not identical, but the genes encode an identical enzyme having acyl-CoA desaturase 1 activity. In some embodiments, the copies of the heterologous gene are not identical, and the genes encode enzymes having acyl-CoA desaturase 1 activity that are different (e.g., mutants, variants, fragments thereof).

In some embodiments, the yeast cell is diploid and one copy of a heterologous gene encoding an enzyme with alcohol-O-acyltransferase activity as described herein is introduced into both copies of the yeast genome. In some embodiments, the copies of the heterologous gene are identical. In some embodiments, the copies of the heterologous gene are not identical, but the genes encode an identical enzyme having alcohol-O-acyltransferase activity. In some embodiments, the copies of the heterologous gene are not identical, and the genes encode enzymes having alcohol-O-acyltransferase activity that are different (e.g., mutants, variants, fragments thereof).

In some embodiments, the yeast cell is tetraploid. Tetraploid yeast cells are cells which maintain four complete sets of chromosomes (i.e., a complete set of chromosomes in four copies). In some embodiments, the yeast cell is tetraploid and a copy of a gene encoding an enzyme with fatty acid hydroxylase activity as described herein is introduced into at least one copy of the genome. In some embodiments, the yeast cell is tetraploid and a copy of a gene encoding an enzyme with fatty acid hydroxylase activity as described herein is introduced into more than one copy of the genome. In some embodiments, the yeast cell is tetraploid and a copy of a gene encoding an enzyme with fatty acid hydroxylase activity as described herein is introduced all four copies of the genome. In some embodiments, the copies of the gene encoding an enzyme with fatty acid hydroxylase activity are identical. In some embodiments, the copies of the gene encoding an enzyme with fatty acid hydroxylase activity are not identical, but the genes encode an identical enzyme having fatty acid hydroxylase activity. In some embodiments, the copies of the gene encoding an enzyme with fatty acid hydroxylase activity are not identical, and the genes encode enzymes having fatty acid synthase activity that are different (e.g., mutants, variants, fragments thereof). In some embodiments, the cell contains a gene encoding an enzyme with fatty acid hydroxylase activity, referred to as an endogenous gene, and also contains one or more additional copies of a gene encoding an enzyme with fatty acid hydroxylase activity, which may be the same or different enzyme with fatty acid hydroxylase activity as that encoded by the endogenous gene.

In some embodiments, the yeast cell is tetraploid and a copy of a gene encoding transcription factor (e.g., a deregulated transcription factor) as described herein is introduced into at least one copy of the genome. In some embodiments, the yeast cell is tetraploid and a copy of a gene encoding transcription factor as described herein is introduced into more than one copy of the genome. In some embodiments, the yeast cell is tetraploid and a copy of a gene encoding transcription factor as described herein is introduced all four copies of the genome. In some embodiments, the copies of the gene are identical. In some embodiments, the copies of the gene are not identical, but the genes encode an identical transcription factor or transcription factors having identical or substantially similar activity. In some embodiments, the copies of the gene are not identical, and the genes encode or transcription factors that are different (e.g., mutants, variants, fragments thereof).

In some embodiments, the yeast cell is tetraploid and a copy of a heterologous gene encoding an enzyme with acyl-CoA desaturase 1 activity as described herein is introduced into at least one copy of the genome. In some embodiments, the yeast cell is tetraploid and a copy of a heterologous gene encoding an enzyme with acyl-CoA desaturase 1 activity as described herein is introduced into more than one copy of the genome. In some embodiments, the yeast cell is tetraploid and a copy of a heterologous gene encoding an enzyme with acyl-CoA desaturase 1 activity as described herein is introduced all four copies of the genome. In some embodiments, the copies of the heterologous gene are identical. In some embodiments, the copies of the heterologous gene are not identical, but the genes encode an identical enzyme having acyl-CoA desaturase 1 activity. In some embodiments, the copies of the heterologous gene are not identical, and the genes encode enzymes having acyl-CoA desaturase 1 activity that are different (e.g., mutants, variants, fragments thereof).

In some embodiments, the yeast cell is tetraploid and a copy of a heterologous gene encoding an enzyme with alcohol-O-acyltransferase activity as described herein is introduced into at least one copy of the genome. In some embodiments, the yeast cell is tetraploid and a copy of a heterologous gene encoding an enzyme with alcohol-O-acyltransferase activity as described herein is introduced into more than one copy of the genome. In some embodiments, the yeast cell is tetraploid and a copy of a heterologous gene encoding an enzyme with alcohol-O-acyltransferase activity as described herein is introduced all four copies of the genome. In some embodiments, the copies of the heterologous gene are identical. In some embodiments, the copies of the heterologous gene are not identical, but the genes encode an identical enzyme having alcohol-O-acyltransferase activity. In some embodiments, the copies of the heterologous gene are not identical, and the genes encode enzymes having alcohol-O-acyltransferase activity that are different (e.g., mutants, variants, fragments thereof).

In some embodiments, the growth rate of the modified cell is not substantially impaired relative to a wild-type yeast cell that does not comprise the first heterologous gene and second exogenous gene. Methods of measuring and comparing the growth rates of two cells will be known to one of ordinary skill in the art. Non-limiting examples of growth rates that can be measured and compared between two types of cells are replication rate, budding rate, colony-forming units (CFUs) produced per unit of time, and amount of fermentable sugar reduced in a medium per unit of time. The growth rate of a modified cell is “not substantially impaired” relative to a wild-type cell if the growth rate, as measured, is at least 70%, at least 75%, at least 80%, at least 85%, at least 90%, at least 95%, at least 99%, or at least 100% of the growth rate of the wild-type cell.

Strains of yeast cells that may be used with the methods described herein will be known to one of ordinary skill in the art and include yeast strains used for brewing desired fermented beverages as well as commercially available yeast strains. Examples of common beer strains include, without limitation, American ale strains, Belgian ale strains, British ale strains, Belgian lambic/sour ale strains, Barleywine/Imperial Stout strains, India Pale Ale strains, Brown Ale strains, Kolsch and Altbier strains, Stout and Porter strains, and Wheat beer strains.

Non-limiting examples of strains for use with the genetically modified cells and methods described herein include Wyeast American Ale 1056, Wyeast American Ale 111272, Wyeast Denny's Favorite 50 1450, Wyeast Northwest Ale 1332, Wyeast Ringwood Ale 1187, Siebel Inst. American Ale BRY 96, White Labs American Ale Yeast Blend WLP060, White Labs California Ale V WLP051, White Labs California Ale WLP001, White Labs Old Sonoma Ale WLP076, White Labs Pacific Ale WLP041, White Labs East Coast Ale WLP008, White Labs East Midlands Ale WLP039, White Labs San Diego Super Yeast WLP090, White Labs San Francisco Lager WLP810, White Labs Neutral Grain WLP078, Lallemand American West Coast Ale BRY-97, Lallemand CBC-1 (Cask and Bottle Conditioning), Brewferm Top, Coopers Pure Brewers' Yeast, Fermentis US-05, Real Brewers Yeast Lucky #7, Muntons Premium Gold, Muntons Standard Yeast, East Coast Yeast Northeast Ale ECY29, East Coast Yeast Old Newark Ale ECY10, East Coast Yeast Old Newark Beer ECY12, Fermentis Safale US-05, Fermentis Safbrew T-58, Real Brewers Yeast The One, Mangrove Jack US West Coast Yeast, Mangrove Jack Workhorse Beer Yeast, Lallemand Abbaye Belgian Ale, White Labs Abbey IV WLP540, White Labs American Farmhouse Blend WLP670, White Labs Antwerp Ale WLP515, East Coast Yeast Belgian Abbaye ECY09, White Labs Belgian Ale WLP550, Mangrove Jack Belgian Ale Yeast, Wyeast Belgian Dark Ale 3822-PC, Wyeast Belgian Saison 3724, White Labs Belgian Saison I WLP565, White Labs Belgian Saison II WLP566, White Labs Belgian Saison III WLP585, Wyeast Belgian Schelde Ale 3655-PC, Wyeast Belgian Stout 1581-PC, White Labs Belgian Style Ale Yeast Blend WLP575, White Labs Belgian Style Saison Ale Blend WLP568, East Coast Yeast Belgian White ECYl 1, Lallemand Belle Saison, Wyeast Biere de Garde 3725-PC, White Labs Brettanomyces Bruxellensis Trois Vrai WLP648, Brewferm Top, Wyeast Canadian/Belgian Ale 3864-PC, Lallemand CBC-1 (Cask and Bottle Conditioning), Wyeast Farmhouse Ale 3726-PC, East Coast Yeast Farmhouse Brett ECY03, Wyeast Flanders Golden Ale 3739-PC, White Labs Flemish Ale Blend WLP665, White Labs French Ale WLP072, Wyeast French Saison 3711, Wyeast Leuven Pale Ale 3538-PC, Fermentis Safbrew T-58, East Coast Yeast Saison Brasserie Blend ECY08, East Coast Yeast Saison Single-Strain ECY14, Real Brewers Yeast The Monk, Siebel Inst. Trappist Ale BRY 204, East Coast Yeast Trappist Ale ECY13, White Labs Trappist Ale WLP500, Wyeast Trappist Blend 3789-PC, Wyeast British Ale 1098, Wyeast British Ale 111335, Wyeast British Cask Ale 1026-PC, Wyeast English Special Bitter 1768-PC, Wyeast Irish Ale 1084, Wyeast London Ale 1028, Wyeast London Ale III 1318, Wyeast London ESB Ale 1968, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, Mangrove Jack British Ale Yeast, Mangrove Jack Burton Union Yeast, Mangrove Jack Workhorse Beer Yeast, East Coast Yeast British Mild Ale ECY18, East Coast Yeast Northeast Ale ECY29, East Coast Yeast Burton Union ECY17, East Coast Yeast Old Newark Ale ECY10, White Labs Bedford British Ale WLP006, White Labs British Ale WLP005, White Labs Burton Ale WLP023, White Labs East Midlands Ale WLP039, White Labs English Ale Blend WLP085, White Labs English Ale WLP002, White Labs Essex Ale Yeast WLP022, White Labs Irish Ale WLP004, White Labs London Ale WLP013, White Labs Manchester Ale WLP038, White Labs Old Sonoma Ale WLP076, White Labs San Diego Super Yeast WLP090, White Labs Whitbread Ale WLP017, White Labs North Yorkshire Ale WLP037, Coopers Pure Brewers' Yeast, Siebel Inst. English Ale BRY 264, Muntons Premium Gold, Muntons Standard Yeast, Lallemand Nottingham, Fermentis Safale S-04, Fermentis Safbrew T-58, Lallemand Windsor (British Ale), Real Brewers Yeast Ye Olde English, Brewferm Top, White Labs American Whiskey WLP065, White Labs Dry English Ale WLP007, White Labs Edinburgh Ale WLP028, Fermentis Safbrew S-33, Wyeast Scottish Ale 1728, East Coast Yeast Scottish Heavy ECY07, White Labs Super High Gravity WLP099, White Labs Whitbread Ale WLP017, Wyeast Belgian Lambic Blend 3278, Wyeast Belgian Schelde Ale 3655-PC, Wyeast Berliner-Weisse Blend 3191-PC, Wyeast Brettanomyces Bruxellensis 5112, Wyeast Brettanomyces Lambicus 5526, Wyeast Lactobacillus 5335, Wyeast Pediococcus Cerevisiae 5733, Wyeast Roeselare Ale Blend 3763, Wyeast Trappist Blend 3789-Pc, White Labs Belgian Sour Mix Wlp655, White Labs Berliner Weisse Blend Wlp630, White Labs Saccharomyces “Bruxellensis” Trois Wlp644, White Labs Brettanomyces Bruxellensis Wlp650, White Labs Brettanomyces Claussenii Wlp645, White Labs Brettanomyces Lambicus Wlp653, White Labs Flemish Ale Blend Wlp665, East Coast Yeast Berliner Blend Ecy06, East Coast Yeast Brett Anomala Ecy04, East Coast Yeast Brett Bruxelensis Ecy05, East Coast Yeast Brett Custersianus Ecy19, East Coast Yeast Brett Nanus Ecy16, Strain #2, East Coast Yeast BugCounty ECY20, East Coast Yeast BugFarm ECY01, East Coast Yeast Farmhouse Brett ECY03, East Coast Yeast Flemish Ale ECYO2, East Coast Yeast Oud Brune ECY23, Wyeast American Ale 1056, Siebel Inst. American Ale BRY 96, White Labs American Ale Yeast Blend WLP060, White Labs Bourbon Yeast WLP070, White Labs California Ale V WLP051, White Labs California Ale WLP001, White Labs Dry English ale WLP007, White Labs East Coast Ale WLP008, White Labs Neutral Grain WLP078, White Labs Super High Gravity WLP099, White Labs Tennessee WLP050, Fermentis US-05, Real Brewers Yeast Lucky #7, Fermentis Safbrew S-33, East Coast Yeast Scottish Heavy ECY07, Lallemand Windsor (British Ale), Wyeast American Ale 1056, Wyeast American Ale 111272, Wyeast British Ale 1098, Wyeast British Ale 111335, Wyeast Denny's Favorite 50 1450, Wyeast London Ale 1028, Wyeast London Ale III 1318, Wyeast London ESB Ale 1968, Wyeast Northwest Ale 1332, Wyeast Ringwood Ale 1187, Siebel Inst. American Ale BRY 96, White Labs American Ale Yeast Blend WLP060, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs English Ale WLP002, White Labs London Ale WLP013, White Labs Essex Ale Yeast WLP022, White Labs Pacific Ale WLP041, White Labs San Diego Super Yeast WLP090, White Labs Whitbread Ale WLP017, Brewferm Top, Mangrove Jack Burton Union Yeast, Mangrove Jack US West Coast Yeast, Mangrove Jack Workhorse Beer Yeast, Coopers Pure Brewers' Yeast, Fermentis US-05, Fermentis Safale S-04, Fermentis Safbrew T-58, Real Brewers Yeast Lucky #7, Real Brewers Yeast The One, Muntons Premium Gold, Muntons Standard Yeast, East Coast Yeast Northeast Ale ECY29, Lallemand Nottingham, Lallemand Windsor (British Ale), Wyeast American Ale 1056, Wyeast American Ale 111272, Wyeast British Ale 1098, Wyeast British Ale 111335, Wyeast Thames Valley Ale 1275, Wyeast Thames Valley Ale II 1882-PC, Wyeast West Yorkshire Ale 1469, Wyeast Whitbread Ale 1099, Wyeast British Cask Ale 1026-PC, Wyeast English Special Bitter 1768-PC, Wyeast London Ale 1028, Wyeast London Ale III 1318, Wyeast London ESB Ale 1968, Wyeast Northwest Ale 1332, Wyeast Ringwood Ale 1187, White Labs American Ale Yeast Blend WLP060, White Labs British Ale WLP005, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs English Ale WLP002, White Labs Essex Ale Yeast WLP022, White Labs French Ale WLP072, White Labs London Ale WLP013, White Labs Pacific Ale WLP041, White Labs Whitbread Ale WLP017, Brewferm Top, East Coast Yeast British Mild Ale ECY18, Coopers Pure Brewers' Yeast, Muntons Premium Gold, Muntons Standard Yeast, Mangrove Jack Newcastle Dark Ale Yeast, Lallemand CBC-1 (Cask and Bottle Conditioning), Lallemand Nottingham, Lallemand Windsor (British Ale), Fermentis Safale S-04, Fermentis US-05, Siebel Inst. American Ale BRY 96, Wyeast American Wheat 1010, Wyeast German Ale 1007, Wyeast Kolsch 2565, Wyeast Kolsch II 2575-PC, White Labs Belgian Lager WLP815, White Labs Dusseldorf Alt WLP036, White Labs European Ale WLP011, White Labs German Ale/Kolsch WLP029, East Coast Yeast Kolschbier ECY21, Mangrove Jack Workhorse Beer Yeast, Siebel Inst. Alt Ale BRY 144, Wyeast American Ale 1056, Wyeast American Ale 111272, Wyeast British Ale 1098, Wyeast British Ale 111335, Wyeast Denny's Favorite 50 1450, Wyeast English Special Bitter 1768-PC, Wyeast Irish Ale 1084, Wyeast London Ale 1028, Wyeast London Ale III 1318, Wyeast London ESB Ale 1968, Wyeast Northwest Ale 1332, Wyeast Ringwood Ale 1187, Wyeast Thames Valley Ale 1275, Wyeast Thames Valley Ale II 1882-PC, Wyeast West Yorkshire Ale 1469, Wyeast Whitbread Ale 1099, White Labs American Ale Yeast Blend WLP060, White Labs Bedford British Ale WLP006, White Labs British Ale WLP005, White Labs Burton Ale WLP023, White Labs California Ale V WLP051, White Labs California Ale WLP001, White Labs East Coast Ale WLP008, White Labs East Midlands Ale WLP039, White Labs English Ale WLP002, White Labs Essex Ale Yeast WLP022, White Labs Irish Ale WLP004, White Labs London Ale WLP013, White Labs Old Sonoma Ale WLP076, White Labs Pacific Ale WLP041, White Labs Whitbread Ale WLP017, Coopers Pure Brewers' Yeast, Fermentis US-05, Muntons Premium Gold, Muntons Standard Yeast, Fermentis Safale S-04, Lallemand Nottingham, Lallemand Windsor (British Ale), Siebel Inst. American Ale BRY 96, White Labs American Hefeweizen Ale 320, White Labs Bavarian Weizen Ale 351, White Labs Belgian Wit Ale 400, White Labs Belgian Wit Ale 11410, White Labs Hefeweizen Ale 300, White Labs Hefeweizen IV Ale 380, Wyeast American Wheat 1010, Wyeast Bavarian Wheat 3638, Wyeast Bavarian Wheat Blend 3056, Wyeast Belgian Ardennes 3522, Wyeast Belgian Wheat 3942, Wyeast Belgian Witbier 3944, Wyeast Canadian/Belgian Ale 3864-PC, Wyeast Forbidden Fruit Yeast 3463, Wyeast German Wheat 3333, Wyeast Weihenstephan Weizen 3068, Siebel Institute Bavarian Weizen BRY 235, Fermentis Safbrew WB-06, Mangrove Jack Bavarian Wheat, Lallemand Munich (German Wheat Beer), Brewferm Blanche, Brewferm Lager, East Coast Yeast Belgian White ECY11. In some embodiments, the yeast is S. cerevisiae strain WLP001.

In some embodiments, the yeast strain for use with the genetically modified cells and methods described herein is a wine yeast strain. Examples of yeast strains for use with the genetically modified cells and methods described herein include, without limitation, Red Star Montrachet, EC-1118, Elegance, Red Star Côte des Blancs, Epernay II, Red Star Premier Cuvee, Red Star Pasteur Red, Red Star Pasteur Champagne, Fermentis BCS-103, and Fermentis VR44. In some embodiments, the yeast is S. cerevisiae strain Elegance.

In some embodiments, the yeast strain is not Yarrowia lipolytica.

### Methods

Aspects of the present disclosure relate to methods of producing a fermented product using any of the genetically modified yeast cells described herein. Also provided are methods of producing ethanol using any of the genetically modified yeast cells described herein.

The process of fermentation exploits a natural process of using microorganisms to convert carbohydrates into alcohol and carbon dioxide. It is a metabolic process that produces chemical changes in organic substrates through enzymatic action. In the context of food production, fermentation broadly refers to any process in which the activity of microorganisms brings about a desirable change to a food product or beverage. The conditions for fermentation and the carrying out of a fermentation is referred to herein as a “fermentation process.”

In some aspects, the disclosure relates to a method of producing a fermented product, such as a fermented beverage, involving contacting any of the modified cells described herein with a medium comprising at least one fermentable sugar during a first fermentation process, to produce a fermented product. A “medium” as used herein, refers to liquid conducive to fermentation, meaning a liquid which does not inhibit or prevent the fermentation process. In some embodiments, the medium is water.

As also used herein, the term “fermentable sugar” refers to a carbohydrate that may be converted into an alcohol and carbon dioxide by a microorganism, such as any of the cells described herein. In some embodiments, the fermentable sugar is converted into an alcohol and carbon dioxide by an enzyme, such as a recombinant enzyme or a cell that expresses the enzyme. Examples of fermentable sugars include, without limitation, glucose, fructose, lactose, sucrose, maltose, and maltotriose.

In some embodiments, the fermentable sugar is provided in a sugar source. The sugar source for use in the claimed methods may depend, for example, on the type of fermented product and the fermentable sugar. Examples of sugar sources include, without limitation, wort, grains/cereals, fruit juice (e.g., grape juice and apple juice/cider), honey, cane sugar, rice, and koji. Examples of fruits from which fruit juice can be obtained include, without limitation, grapes, apples, blueberries, blackberries, raspberries, currants, strawberries, cherries, pears, peaches, nectarines, oranges, pineapples, mangoes, and passionfruit.

Aspects of the present disclosure relate to modified cells that are capable of producing levels of γ-decalactone that are above the odor threshold in a particular medium for a human subject. As will be appreciated by one of ordinary skill in the art, the odor threshold of γ-decalactone may vary depending on the medium, e.g., wine or beer as compared to water. For example, the odor threshold of γ-decalactone in wine is about 35 μg/L for human subjects. In some embodiments, the modified cells are capable of producing γ-decalactone levels of at least 35 μg/L. As described herein, fermentation using the modified cells described herein is performed in the presence of one or more fermentable sugars. In some embodiments, fermentation using the modified cells described herein is performed in the absence of intermediate molecules of the γ-decalactone biosynthesis pathways. In some embodiments, fermentation using the modified cells described herein is performed in the absence of fatty acid intermediates of the γ-decalactone biosynthesis pathways. In some embodiments, fermentation using the modified cells described herein is performed in the absence of oleic acid or ricinoleic acid in the medium.

In some embodiments, the medium comprising the fermentable sugar is pre-oxygenated. As will be evident to one of ordinary skill in the art, pre-oxygenation is the process of introducing oxygen gas to a culture medium to increase available oxygen for the microorganism in culture. In some embodiments, the culture medium is pre-oxygenated prior to inoculation with yeast. Microorganisms inoculated into a pre-oxygenated medium rapidly consume the available oxygen and are able to increase production of fermentation products.

In some embodiments, the modified cells described herein are cultured in an anaerobic or semi-anaerobic environment. Anaerobic cell culture refers to the technique of culturing a microorganism, such as a modified yeast cell, in an environment without available oxygen. Semi-anaerobic cell culture refers to the technique of culturing a microorganism, such as a modified yeast cell, in an environment with limited oxygen availability, such as in a medium that has been pre-oxygenated. In some embodiments, the modified cells described herein are not cultured in an anaerobic environment.

In some embodiments, the modified cells described herein are cultured in an aerobic environment. In some embodiments, the modified cells described herein are cultured in an aerobic environment for a period of time, such that oxygen availability is limited temporally. In some embodiments, the modified cells described herein are cultured in an aerobic environment for a portion of the fermentation process. In some embodiments, the modified cells described herein are cultured in an aerobic environment for at least 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, or longer. In some embodiments, the modified cells described herein are cultured in an aerobic environment for a portion of the fermentation process followed by culturing in an anaerobic environment for a portion of the fermentation process.

In some embodiments, the modified cells described herein are cultured in an aerobic environment for a portion of the fermentation process followed by culturing in an anerobic environment for a portion of the fermentation process.

As will be evident to one of ordinary skill in the art, in some instances, it may be necessary to process the sugar source in order to make available the fermentable sugar for fermentation. Using beer production as an example fermented beverage, grains (cereal, barley) are boiled or steeped in water, which hydrates the grain and activates the malt enzymes converting the starches to fermentable sugars, referred to as “mashing.” As used herein, the term “wort” refers to the liquid produced in the mashing process, which contains the fermentable sugars. The wort then is exposed to a fermenting organism (e.g., any of the cells described herein), which allows enzymes of the fermenting organism to convert the sugars in the wort to alcohol and carbon dioxide.

In some embodiments, the grains are malted, unmalted, or comprise a combination of malted and unmalted grains. Examples of grains for use in the methods described herein include, without limitation, barley, oats, maize, rice, rye, sorghum, wheat, karasumugi, and hatomugi.

In the example of producing sake, the sugar source is rice, which is incubated with koji mold (Aspergillus oryzae) converting the rice starch to fermentable sugar, producing koji. The koji then is exposed to a fermenting organism (e.g., any of the cells described herein), which allows enzymes of the fermenting organism to convert the sugars in the koji to alcohol and carbon dioxide.

In the example of producing wine, grapes are harvested, mashed (e.g., crushed) into a composition containing the skins, solids, juice, and seeds. The resulting composition is referred to as the “must.” The grape juice may be separated from the must and fermented, or the entirety of the must (i.e., with skins, seeds, solids) may be fermented. The grape juice or must is then exposed to a fermenting organism (e.g., any of the cells described herein), which allows enzymes of the fermenting organism to convert the sugars in the grape juice or must to alcohol and carbon dioxide.

In some embodiments, the methods described herein involve producing the medium, which may involve heating or steeping a sugar source, for example in water. In some embodiments, the water has a temperature of at least 50 degrees Celsius (50° C.) and incubated with a sugar source of a period of time. In some embodiments, the water has a temperature of at least 75° C. and incubated with a sugar source of a period of time. In some embodiments, the water has a temperature of at least 100° C. and incubated with a sugar source of a period of time. Preferably, the medium is cooled prior to addition of any of the cells described herein.

In some embodiments, the methods described herein further comprise adding at least one (e.g., 1, 2, 3, 4, 5, or more) hop variety, for example to the medium, to a wort during a fermentation process. Hops are the flowers of the hops plant (Humulus lupulus) and are often used in fermentation to impart various flavors and aromas to the fermented product. Hops are considered to impart bitter flavoring in addition to floral, fruity, and/or citrus flavors and aromas and may be characterized based on the intended purpose. For example, bittering hops impart a level of bitterness to the fermented product due to the presence of alpha acids in the hop flowers, whereas aroma hops have lower lowers of alpha acids and contribute desirable aromas and flavor to the fermented product.

Whether one or more variety of hops is added to the medium and/or the wort and at stage during which the hops are added may be based on various factors, such as the intended purpose of the hops. For example, hops that are intended to impart a bitterness to the fermented product are typically added to during preparation of the wort, for example during boiling of the wort. In some embodiments, hops that are intended to impart a bitterness to the fermented product are added to the wort and boiled with the wort for a period of time, for example, for about 15-60 minutes. In contrast, hops that are intended to impart desired aromas to the fermented product are typically added later than hops used for bitterness. In some embodiments, hops that are intended to impart desired aromas to the fermented product are added to at the end of the boil or after the wort is boiled (i.e., “dry hopping”). In some embodiments, one or more varieties of hops may be added at multiple times (e.g., at least twice, at least three times, or more) during the method.

In some embodiments, the hops are added in the form of either wet or dried hops and may optionally be boiled with the wort. In some embodiments, the hops are in the form of dried hop pellets. In some embodiments, at least one variety of hops is added to the medium. In some embodiments, the hops are wet (i.e., undried). In some embodiment, the hops are dried, and optionally may be further processed prior to use. In some embodiments, the hops are added to the wort prior to the fermentation process. In some embodiments, the hops are boiled in the wort. In some embodiments, the hops are boiled with the wort and then cooled with the wort.

Many varieties of hops are known in the art and may be used in the methods described herein. Examples of hop varieties include, without limitation, Ahtanum, Amarillo, Apollo, Cascade, Centennial, Chinook, Citra, Cluster, Columbus, Crystal/Chrystal, Eroica, Galena, Glacier, Greenburg, Horizon, Liberty, Millennium, Mosaic, Mount Hood, Mount Rainier, Newport, Nugget, Palisade, Santiam, Simcoe, Sterling, Summit, Tomahawk, Ultra, Vanguard, Warrior, Willamette, Zeus, Admiral, Brewer's Gold, Bullion, Challenger, First Gold, Fuggles, Goldings, Herald, Northdown, Northern Brewer, Phoenix, Pilot, Pioneer, Progress, Target, Whitbread Golding Variety (WGV), Hallertau, Hersbrucker, Saaz, Tettnang, Spalt, Feux-Coeur Francais, Galaxy, Green Bullet, Motueka, Nelson Sauvin, Pacific Gem, Pacific Jade, Pacifica, Pride of Ringwood, Riwaka, Southern Cross, Lublin, Magnum, Perle, Polnischer Lublin, Saphir, Satus, Select, Strisselspalt, Styrian Goldings, Tardif de Bourgogne, Tradition, Bravo, Calypso, Chelan, Comet, El Dorado, San Juan Ruby Red, Sonnet Golding, Super Galena, Tillicum, Bramling Cross, Pilgrim, Hallertauer Herkules, Hallertauer Magnum, Hallertauer Taurus, Merkur, Opal, Smaragd, Halleratau Aroma, Kohatu, Rakau, Stella, Sticklebract, Summer Saaz, Super Alpha, Super Pride, Topaz, Wai-iti, Bor, Junga, Marynka, Premiant, Sladek, Styrian Atlas, Styrian Aurora, Styrian Bobek, Styrian Celeia, Sybilla Sorachi Ace, Hallertauer Mittelfrueh, Hallertauer Tradition, Tettnanger, Tahoma, Triple Pearl, Yakima Gold, and Michigan Copper.

In some embodiments, the fermentation process of at least one sugar source comprising at least one fermentable sugar may be carried out for about 1 day to about 31 days. In some embodiments, the fermentation process is performed for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days or longer. In some embodiments, the fermentation process of the one or more fermentable sugars may be performed at a temperature of about 4° C. to about 30° C. In some embodiments, the fermentation process of one or more fermentable sugars may be carried out at temperature of about 8° C. to about 14° C. or about 18° C. to about 24° C. In some embodiments, the fermentation process of one or more fermentable sugars may be performed at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

In some embodiments, fermentation results in the reduction of the amount of fermentable sugar present in a medium. In some embodiments, the reduction in the amount of fermentable sugar occurs within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, or longer, from the start of fermentation. In some embodiments, the amount of fermentable sugar is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, at least 99.5%, at least 99.6%, at least 99.7%, at least 99.8%, at least 99.9%, or 100%. In some embodiments, the modified cell or cells ferment a comparable or greater amount of fermentable sugar, relative to the amount of fermentable sugar fermented by wild-type yeast cells in the same amount of time.

The methods described herein may involve at least one additional fermentation process. Such additional fermentation methods may be referred to as secondary fermentation processes (also referred to as “aging” or “maturing”). As will be understood by one of ordinary skill in the art, secondary fermentation typically involves transferring a fermented beverage to a second receptacle (e.g., glass carboy, barrel) where the fermented beverage is incubated for a period of time. In some embodiments, the secondary fermentation is performed for a period of time between 10 minutes and 12 months. In some embodiments, the secondary fermentation is performed for 10 minutes, 20 minutes, 40 minutes, 40 minutes, 50 minutes, 60 minutes (1 hour), 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 8 days, 9 days, 10 days, 11 days, 12 days, 13 days, 14 days, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 3 months, 4 months, 5 months, 6 months, 7 months, 8 months, 9 months, 10 months, 11 months, 12 months, or longer. In some embodiments, the additional or secondary fermentation process of the one or more fermentable sugars may be performed at a temperature of about 4° C. to about 30° C. In some embodiments, the additional or secondary fermentation process of one or more fermentable sugars may be carried out at temperature of about 8° C. to about 14° C. or about 18° C. to about 24° C. In some embodiments, the additional or secondary fermentation process of one or more fermentable sugars may be performed at a temperature of about 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., or 30° C.

As will be evident to one of ordinary skill in the art, selection of a time period and temperature for an additional or secondary fermentation process will depend on factors such as the type of beer, the characteristics of the beer desired, and the yeast strain used in the methods.

In some embodiments, one or more additional flavor component may be added to the medium prior to or after the fermentation process. Examples include, hop oil, hop aromatics, hop extracts, hop bitters, and isomerized hops extract.

Products from the fermentation process may volatilize and dissipate during the fermentation process or from the fermented product. For example, γ-decalactone produced during fermentation using the cells described herein may volatilize resulting in reduced levels of γ-decalactone in the fermented product. In some embodiments, volatilized γ-decalactone is captured and re-introduced after the fermentation process.

Various refinement, filtration, and aging processes may occur subsequent fermentation, after which the liquid is bottled (e.g., captured and sealed in a container for distribution, storage, or consumption). Any of the methods described herein may further involve distilling, pasteurizing, and/or carbonating the fermented product. In some embodiments, the methods involve carbonating the fermented product. Methods of carbonating fermented beverages are known in the art and include, for example, force carbonating with a gas (e.g., carbon dioxide, nitrogen), naturally carbonating by adding a further sugar source to the fermented beverage to promote further fermentation and production of carbon dioxide (e.g., bottle conditioning).

In some embodiments, the methods involve mixing a fermented product produced by any of the modified cells described herein with a fermented product, e.g., a fermented product produced using cells that have not been modified to express any of the enzymes described herein. In some embodiments, the modified cells described herein are used to produce a product comprising increased levels of γ-decalactone which may subsequently be mixed with a fermented product produced using cells that have not been modified as described herein, for example, to increase the level of γ-decalactone.

### Fermented Products

Aspects of the present disclosure relate to fermented products produced by any of the methods disclosed herein. In some embodiments, the fermented product is a fermented beverage. Examples of fermented beverages include, without limitation, beer, wine, sake, mead, cider, cava, sparkling wine (champagne), kombucha, ginger beer, water kefir. In some embodiments, the beverage is beer. In some embodiments, the beverage is wine. In some embodiments, the beverage is sparkling wine. In some embodiments, the beverage is Champagne. In some embodiments, the beverage is sake. In some embodiments, the beverage is mead. In some embodiments, the beverage is cider. In some embodiments, the beverage is hard seltzer. In some embodiments, the beverage is a wine cooler.

In some embodiments, the fermented product is a fermented food product. Examples of fermented food products include, without limitation, cultured yogurt, tempeh, miso, kimchi, sauerkraut, fermented sausage, bread, and soy sauce.

According to aspects of the invention, increased titers of γ-decalactone are produced through the recombinant expression of genes associated with the invention, in yeast cells and use of the cells in the methods described herein. As used herein, an “increased titer” or “high titer” refers to a titer in the micrograms per liter (μg L-1) scale. The titer produced for a given product will be influenced by multiple factors including the choice of medium and conditions for fermentation.

In some embodiments, the titer of γ-decalactone is at least 1 μg L⁻¹, for example at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 g L-1 or more. In some embodiments, the titer of γ-decalactone is at least 1.05, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10 g L⁻¹ or more.

In some embodiments, the titer of γ-decalactone is detectable to a human subject, e.g., above the odor threshold of a human subject. In some embodiments, the titer of γ-decalactone is at least about 35 μg L⁻¹, which is typically considered to be the odor threshold of human subjects for γ-decalactone in wine.

Aspects of the present disclosure relate to reducing the production of undesired products (e.g., byproducts, off-flavors), such as ethyl acetate, during fermentation of a product. In some embodiments, expression of the any of the enzymes described herein, such as the fatty acid hydroxylases, acyl-CoA desaturase 1, and/or alcohol-O-acyltransferases in the genetically modified cells described herein result in a reduction in the production of an undesired product by about 5%10%, 15%, 20%, 25%, 30%, 35%, 40%, 45%, 50%, 55%, 60%, 65%, 70%, 75%, 80%, 85%, 90%, 95% or more relative to production of the undesired product (e.g., ethyl acetate) by use of a wild-type yeast cell or a yeast cell that does not express the enzymes.

As described herein, the production of ethyl acetate can impart a solvent-like aroma to fermented products. In some embodiments, the titer of ethyl acetate is less than 1000 mg L⁻¹, for example less than 1000, 950, 900, 850, 800, 750, 700, 650, 600, 550, 500, 450, 400, 350, 300, 250, 200, 150, 100, 95, 90, 85, 80, 75, 70, 65, 60, 55, 50, 45, 40, 35, 30, 25, 20, 15, 10, 9, 8, 7, 6, 5, 4, 3, 2, 1, 0.9, 0.8, 0.7, 0.6, 0.5, 0.4, 0.3, 0.2, 0.1 mg L⁻¹ or less. In some embodiments, the titer of ethyl acetate is below the limit of human detection.

Methods of measuring titers/levels of γ-decalactone and/or ethyl acetate will be evident to one of ordinary skill in the art. In some embodiments, the titers/levels of γ-decalactone and/or ethyl acetate are measured using gas-chromatograph mass-spectrometry (GC/MS). In some embodiments, the titers/levels of γ-decalactone and/or ethyl acetate are assessed using sensory panels, including for example human taste-testers.

In some embodiments, the fermented beverage contains an alcohol by volume (also referred to as “ABV,” “abv,” or “alc/vol”) between 0.1% and 30%. In some embodiments, the fermented beverage contains an alcohol by volume of about 0.1%, 0.2%, 0.3%, 0.4%, 0.5%, 0.6%, 0.07%, 0.8%, 0.9%, 1.0%, 1.1%, 1.2%, 1.3%, 1.4%, 1.5%, 1.6%, 1.7%, 1.8%, 1.9%, 20%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or higher. In some embodiments, the fermented beverage is non-alcoholic (e.g., has an alcohol by volume less than 0.5%).

### Kits

Aspects of the present disclosure also provides kits for use of the genetically modified yeast cells, for example to produce a fermented beverage, fermented product, or ethanol. In some embodiments, the contains a modified cell containing a heterologous gene encoding an enzyme with fatty acid hydroxylase (FAH) activity.

In some embodiments, the modified yeast cells express a heterologous gene encoding an enzyme having fatty acid hydroxylase (FAH) activity and a gene encoding a deregulated transcription factor (e.g., ADR1). In some embodiments, the modified yeast cells express a heterologous gene encoding an enzyme having fatty acid hydroxylase (FAH) activity, a gene encoding a deregulated transcription factor (e.g., ADR1), and a gene encoding an enzyme having acyl-CoA desaturase 1 (OLE1) activity. In some embodiments, the modified yeast cells express a heterologous gene encoding an enzyme having fatty acid hydroxylase (FAH) activity and a gene encoding an enzyme having acyl-CoA desaturase 1 (OLE1) activity. In some embodiments, the modified yeast cells express a heterologous gene encoding an enzyme having fatty acid hydroxylase (FAH) activity, a gene encoding an enzyme having acyl-CoA desaturase 1 (OLE1) activity, and a heterologous gene encoding an enzyme having alcohol-O-acyltransferase (AAT) activity. In some embodiments, the modified yeast cells express a heterologous gene encoding an enzyme having fatty acid hydroxylase (FAH) activity, a gene encoding a deregulated transcription factor (e.g., ADR1), a gene encoding an enzyme having acyl-CoA desaturase 1 (OLE1) activity, and a heterologous gene encoding an enzyme having alcohol-O-acyltransferase (AAT) activity. In some embodiments, the modified yeast cells express a heterologous gene encoding an enzyme having fatty acid hydroxylase (FAH) activity and a heterologous gene encoding an enzyme having alcohol-O-acyltransferase (AAT) activity.

In some embodiments, the kit contains a modified yeast cell that expresses a heterologous gene encoding an enzyme having fatty acid hydroxylase (FAH) activity and a deregulated transcription factor, such as ADR (e.g., ADR S230A). In some embodiments, the kit contains a modified yeast cell that expresses a heterologous gene encoding an enzyme having fatty acid hydroxylase (FAH) activity, a gene encoding an enzyme having acyl-CoA desaturase 1 (OLE1) activity, and a gene encoding a deregulated transcription factor, such as ADR (e.g., ADR S230A). In some embodiments, the kit contains a modified yeast cell that expresses a heterologous gene encoding an enzyme having fatty acid hydroxylase (FAH) activity, a gene encoding an enzyme having acyl-CoA desaturase 1 (OLE1) activity, a heterologous gene encoding an enzyme having alcohol-O-acyltransferase (AAT) activity, and a gene encoding a deregulated transcription factor, such as ADR (e.g., ADR S230A).

In some embodiments, the kit is for the production of a fermented beverage. In some embodiments, the kit is for the production of beer. In some embodiments, the kit is for the production of wine. In some embodiments, the kit is for the production of sake. In some embodiments, the kit is for the production of mead. In some embodiments, the kit is for the production of cider.

The kits may also comprise other components for use in any of the methods described herein, or for use of any of the cells as described herein. For example, in some embodiments, the kits may contain grains, water, wort, must, yeast, hops, juice, or other sugar source(s). In some embodiments, the kit may contain one or more fermentable sugars. In some embodiments, the kit may contain one or more additional agents, ingredients, or components.

Instructions for performing the methods described herein may also be included in the kits described herein.

The kits may be organized to indicate a single-use compositions containing any of the modified cells described herein. For example, the single use compositions (e.g., amount to be used) can be packaged compositions (e.g., modified cells) such as packeted (i.e., contained in a packet) powders, vials, ampoules, culture tube, tablets, caplets, capsules, or sachets containing liquids.

The compositions (e.g., modified cells) may be provided in dried, lyophilized, frozen, or liquid forms. In some embodiments, the modified cells are provided as colonies on an agar medium. In some embodiments, the modified cells are provided in the form of a starter culture that may be pitched directly into a medium. When reagents or components are provided as a dried form, reconstitution generally is by the addition of a solvent, such as a medium. The solvent may be provided in another packaging means and may be selected by one skilled in the art.

A number of packages or kits are known to those skilled in the art for dispensing a composition (e.g., modified cells). In certain embodiments, the package is a labeled blister package, dial dispenser package, tube, packet, drum, or bottle.

Any of the kits described herein may further comprise one or more vessel for performing the methods described herein, such as a carboy or barrel.

### General Techniques

The practice of the subject matter of the disclosure will employ, unless otherwise indicated, conventional techniques of molecular biology (including recombinant techniques), microbiology, cell biology, biochemistry, and immunology, which are within the skill of the art. Such techniques are explained fully in the literature, such as, but without limiting, Molecular Cloning: A Laboratory Manual, J. Sambrook, et al., eds., Fourth Edition, Cold Spring Harbor Laboratory Press, Cold Spring Harbor, New York, 2012; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999).

### EQUIVALENTS AND SCOPE

It is to be understood that this disclosure is not limited to any or all of the particular embodiments described expressly herein, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this disclosure are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc.) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

### EXAMPLES

### Example 1

Several groups have attempted to engineer yeast strains for increased production of γ-decalactone during the fermentation process. However, these efforts have yet to produce commercially viable yeast with enhanced γ-decalactone production, primarily due to challenges in balancing strain phenotypes of increasing production of γ-decalactone, unaltered growth rate. Furthermore, the most used strain for γ-decalactone production, Yarrowia lipolytica, is unable to produce ricinoleic acid from oleic acid, a critical step in γ-decalactone biosynthesis. Due to the inability of Y. lipolytica to produce ricinoleic acid from oleic acid, most studies attempting to utilize this yeast for γ-decalactone biosynthesis have supplied ricinoleic acid by adding it to the growth medium. In order to maximize flux of ricinoleic acid through the beta-oxidation pathway, these studies have used ricinoleic acid as a sole carbon source, thus forcing the yeast to upregulate beta-oxidation as a means to generate acetyl-CoA required for growth. Growth of wild-type Y. lipolytica with methyl ricinoleate as the sole carbon source has been shown to produce up to 1 g/L γ-decalactone (see, e.g., Wache, et al., J. Mol. Catalysis B: Enzymatic (2002) 19-20 347-351; Gomes, et al., Biocat. and Biotransformation. (2010) 28 227-234). Other studies that have utilized castor oil (composed of 90% ricinoleic acid) as a source of ricinoleic acid have been able to increase γ-decalactone production by wild-type Y. lipolytica as high as 3.5 g/L in batch culture (, or 11 g/L in bioreactor conditions (see, e.g., Soares, et al., Prep. Biochem. Biotechnol. (2017) 47: 633-637; Malajowicz, et al., Biotechnology & Biotechnological Equipment. (2020) 34: 330-340; Krzyczkowska, et al., Chem. Technol. (2012) 61(3): 58-61; U.S. Pat. No. 6,451,565).

Other groups have sought to increase γ-decalactone production by Y. lipolytica through genetic engineering of the beta-oxidation pathway. These efforts generally sought to reduce beta-oxidation of C10 and shorter acyl-CoA molecules, as this leads to an increase in the pool of C10 fatty acids, including 4-hydroxydecanoic acid. In 2000, Wache et al. deleted POX3, a gene that encodes a short-chain specific acyl CoA oxidase responsible for continued oxidation of 4-hydroxydecanoic acid. When grown on 5 g/L methyl ricinoleate, the resulting strain produced 220 mg/L γ-decalactone after 24 hours, a 5-fold increase over the wild type (see, Wache, et al., Appl. Environ. Microbiol. (2000) 66: 1233-1236). The same group went on to delete POX5, a gene that encodes an acyl CoA oxidase with weak activity on short-chain acyl CoAs, and overexpress POX2, a gene that encodes a long-chain specific acyl CoA oxidase. The resulting strain accumulated γ-decalactone over 4 days whereas γ-decalactone production by the wild type peaked at 12 hours and then declined. In 2012, Guo et al. took a similar engineering approach by deleting POX3 in a Y. lipolytica strain overexpressing POX2. This strain produced 3.3 g/L γ-decalactone at 100 hours when grown on 5% w/v methyl ricinoleate (Guo, et al., Microbiol. Res. 167, 246-252 (2012)).

In 2020, Marella et al. expanded upon this prior work and combined the targeted engineering of beta-oxidation with expression of an oleic acid hydratase gene in Y. lipolytica (Marella, et al., Metab. Eng. (2020) 61: 427-436). Expression of the oleic acid hydratase allowed for the production of dodecalactone using oleic acid as an initial pathway substrate or S-decalactone using linoleic acid as an initial pathway substrate. Similar to past studies, the modifications to beta-oxidation in this work sought to inhibit the shortening of acyl-CoA chains of 10 carbons or less. This combination of beta-oxidation engineering and hydratase expression resulted in the generation of a Y. lipolytica strain that produced up to 74.6 mg/L γ-decalactone. Importantly, these experiments relied on the feeding of 30 mg/L oleic acid as a substrate for γ-decalactone production. Marella et al. did not report any data describing the yields of lactones produced without supplementation of fatty acids. The lower concentration of γ-decalactone produced in these experiments compared to prior works that supplied ricinoleic acid is notable, as it suggests that the enzymatic conversion of oleic acid to ricinoleic acid may be a rate limiting step in the pathway.

Also in 2020, an acyltransferase gene was isolated from peach that is capable of catalyzing the lactonization of 4-hydroxydecanoic acid to γ-decalactone (Peng, et al., Plant Physiol. 182, 2065-2080 (2020)). This acyltransferase (PpAAT1), was expressed in Y. lipolytica, after which the engineered strains were immobilized and used to biotransform ricinoleic acid to γ-decalactone. In this context, ˜3.5 g/L γ-decalactone was produced, representing a 7-fold increase over a control strain not expressing the PpAAT1. In contrast, the modified cells described herein are capable of producing increased levels of γ-decalactone, reduced levels of off-flavors (e.g., ethyl acetate), and have substantially unaltered growth characteristics.

### Generation of a Modified Microbe for Improved γ-Decalactone Biosynthesis

To produce peach flavors in fermented beverages, microbial strains were engineered to increase levels of γ-decalactone during beer and wine fermentation. Compared to prior biosynthesis efforts in Y. lipolytica and other fungal hosts, engineering γ-decalactone biosynthesis in wine and beer yeast during beverage fermentation presents several additional challenges. First, Saccharomyces cerevisiae (S. cerevisiae) produces fewer fatty acids than Y. lipolytica, thus limiting flux through the lactone biosynthesis pathway. Second, all prior studies promoted beta-oxidation and lactone formation by growing the host organism on fatty acids that would be used as substrates for lactone formation. This is not feasible during beverage fermentation where the primary carbon sources are hexose sugars, such as glucose, fructose, and maltose. Supplementing beverage fermentations with ricinoleic acid or other fatty acids would be cost prohibitive in obtaining purified hydroxylated fatty acids and challenging due to glucose repression of beta-oxidation. Further, Y. lipolytica encodes six acyl-CoA oxidases, each with different chain length specificities, whereas S. cerevisiae only encodes one acyl-CoA oxidase with broad specificity. Therefore, unlike Y. lipolytica, it is not possible to reduce beta-oxidization of the γ-decalactone precursor, 4-hydroxydecanoic acid, simply by deleting the subset of acyl-CoA oxidases that recognize this molecule as a substrate. In addition, beverage fermentations are primarily anaerobic or semi-anaerobe processes. Previous efforts to engineer γ-decalactone biosynthesis have been done in an aerobic environment as oxygen is required for many steps of the γ-decalactone biosynthesis pathway (e.g., fatty acid desaturation, fatty acid hydroxylation, and beta-oxidation).

In an effort to engineer a white wine yeast strain to produce γ-decalactone, as a first step, production of oleic acid, precursor to γ-decalactone, was increased. Oleic acid is found at low concentrations in S. cerevisiae. To increase the amount of oleic acid available for biosynthesis of γ-decalactone, a nucleic acid encoding an acyl-CoA desaturase 1 (OLE1) enzyme from S. cerevisiae was overexpressed in S. cerevisiae under transcriptional control of the strong promoter pENO2.

OLE1 converts available stearic acid to oleic acid, thus increasing accumulation of oleic acid in S. cerevisiae. To generate γ-decalactone from oleic acid, oleic acid is converted first to ricinoleic acid. Oleic acid can be converted to ricinoleic acid by a fatty acid hydroxylase. Next, the fatty acid hydroxylase (FAH) enzyme from Claviceps purpurea was heterologously overexpressed.

Ricinoleic acid undergoes beta-oxidation thought to occur in the S. cerevisiae peroxisome to produce 4-hydroxydecanoic acid. Finally, a gene encoding an alcohol-O-acyltransferase from a peach plant (Prunus persica; PpAAT1) was introduced into S. cerevisiae to catalyze lactonization of 4-hydroxydecanoic acid to γ-decalactone.

The resulting strain (BY1019), expressing OLE1, FAH, and PpAAT1, was grown aerobically in either a grape juice medium or a synthetic defined yeast medium containing 2% glucose as a carbon source. In both conditions, BY1019 produced a strong peach aroma, the cultures also had a strong solvent aroma, characterized as nail-polish-like, due to levels of ethyl acetate.

### Generation of a Modified Microbe for Increased γ-Decalactone and Decreased Ethyl Acetate

To further increase production of levels of γ-decalactone while also decreasing production of ethyl acetate, PpAAT1 was targeted to the peroxisome organelle, based on the hypothesis that this enzyme contributed to the ethyl acetate production. Briefly, a short peroxisome localization peptide sequence was added to the C-terminus of PpAAT1. Without wishing to be bound by any particular theory, a goal of localizing PpAAT1 to the peroxisome was to increase lactonization of 4-hydroxydecanoic acid to produce γ-decalactone by localizing PpAAT1 to the same compartment as beta-oxidation. To accomplish this, PpAAT1 of strain BY1019 was modified to include a peroxisomal tag, resulting in strain BY1021. This strain was grown aerobically in either a grape juice medium or a yeast medium containing 2% glucose as a carbon source. In both conditions, BY1021 produced a strong peach aroma and a minimally solvent/ethyl acetate associated aroma. Therefore, it was considered that targeting of PpAAT to the peroxisome drastically reduced ethyl acetate production while maintaining similar or greater γ-decalactone production.

### Growing a Modified Microbe to Generate γ-Decalactone During Semi-Anaerobic Fermentation

To determine whether strain BY1019 or BY1021 could produce increased levels of γ-decalactone during semi-anaerobic fermentation conditions that mimic wine-making or brewing conditions, a glucose media was pre-oxygenated by vigorous shaking for about 5 hours. The cultures were inoculated with either BY1019, BY1021, or a wild-type non-engineered strain. The presence of oxygen during the fermentation process allows the yeast to grow vigorously during the early stages of fermentation and is essential for the production of certain beer and wine styles. Although the prolonged bubbling of oxygen into the fermentation would be expected to lead to the production of strong oxidized off flavor molecules, the oxygen introduced by pre-oxygenation is rapidly consumed by the yeast and does not lead to off flavor production. Use of pre-oxygenated cultures may be indicative of whether the strains would be able to produce γ-decalactone during commercial fermentations.

After each strain consumed all fermentable sugars, each culture was assessed for the presence of peach aroma, as well as any off-flavors. The wild-type strain did not produce any peach aroma notes. Strain BY1019 produced a mild peach aroma, while strain BY1021 produced minimal peach aroma. Strain BY1019 ferments had a mild ethyl acetate-like aroma, whereas ferments from strain BY1021 had no perceptible ethyl acetate-like aroma.

Based on these data, it is possible to engineer Saccharomyces cerevisiae to produce γ-decalactone in pre-oxygenated fermentations, similar to the conditions employed in the beer and wine industries. Through modifying genetic or fermentation parameters, the production of ethyl acetate off-flavors can be minimized and titers of γ-decalactone produced by the engineered strains can be increased.

### Example 2

To generate yeast strains capable of improved γ-decalactone production, exemplary Saccharomyces cerevisiae wine yeast strain Elegance was genetically engineered to express oleate 12-hydroxylases obtained from various sources, such as Claviceps purpurea (CpFAH), Hiptage benghalensis (HpFAH), Physaria lindheimeri (PlFAH), Ricinus communis (RcFAH), or Lesquerella fendleri (LFAH12). The oleate 12-hydroxylases were expressed under control of the PGK1 promoter and integrated into the PDC6 genomic locus. See, Table 1.

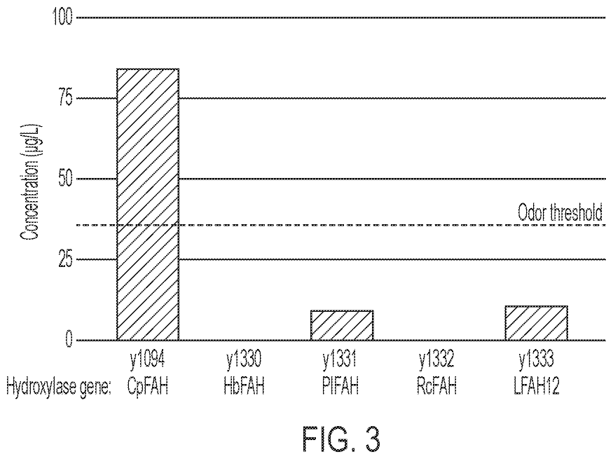

After 24 hours of aerobic growth, samples were assessed and the level of γ-decalactone produced was measured and compared to the odor threshold for human detection. See, FIG. 3.

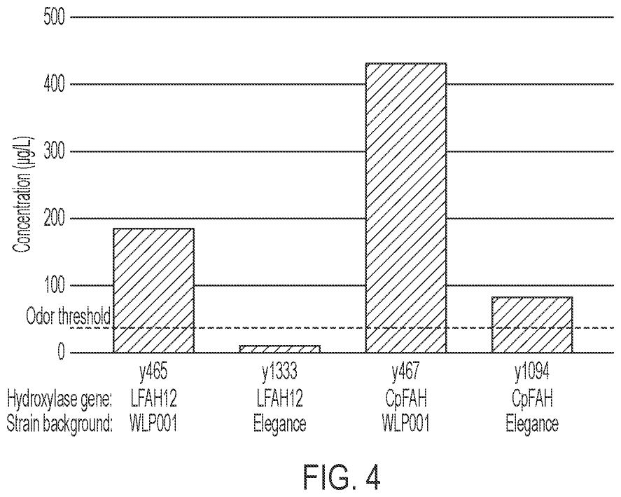

To determine whether expression of the oleate 12-hydroxylases also increased production of γ-decalactone in beer yeast strains, Saccharomyces cerevisiae beer yeast strain as well as wine yeast strain Elegance were engineered to express either LFH12 or CpFAH, under control of the PDGK1 promoter and cultured for 24 hours aerobically. As shown in FIG. 4, expression of CpFAH in either yeast strain resulted in γ-decalactone levels over the odor threshold. Similarly, levels of γ-decalactone were also detected above the odor threshold following expression of LFAH12 in beer strain WLP001.

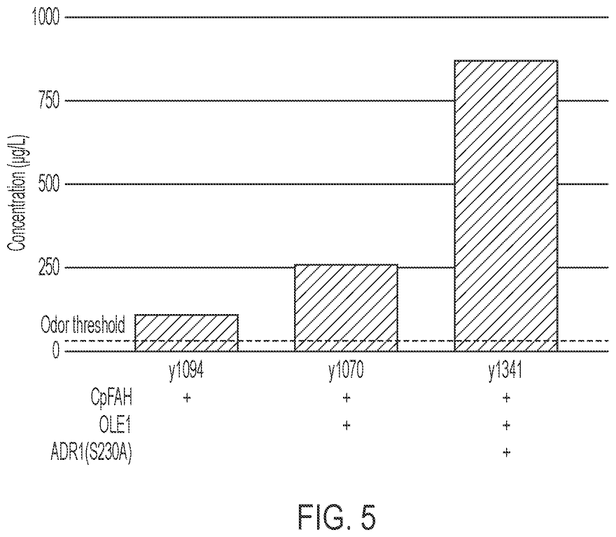

To further increase production of γ-decalactone, several additional genes were evaluated for co-expression along with an oleate 12-hydroxylase in the S. cerevisiae strains. As shown in FIG. 2, in the combination of γ-decalactone biosynthetic pathway, enzymes having OLE1 activity convert available stearic acid to oleic acid, which may increase accumulation of oleic acid in S. cerevisiae. Further, a deregulated mutant of the positive transcriptional activator of glucose-regulated genes, ADR1, was also expressed with the oleate 12-hydroxylase and OLE1 enzymes. After 24 hours of aerobic growth, samples were assessed and the level of γ-decalactone produced was measured and compared to the odor threshold for human detection. It was observed that expression of an enzyme having OLE1 activity increased γ-decalactone levels as compared to expression of the oleate 12-hydroxylase only, which was further increased following the additional expression of the deregulated ADR1 transcription factor. See, FIG. 5.

### Oxygen Availability and Production of γ-Decalactone

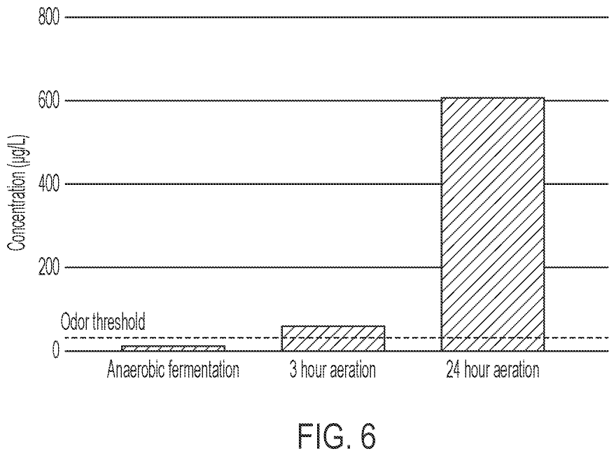

Typically, yeast strains are grown anaerobically to facilitate the process of fermentation. The effect of oxygen availability on production of γ-decalactone by the engineered strains was evaluated. A S. cerevisiae Elegance strain expressing CpFAH, OLE1, MpAAT (N385D V62A) (y1185) was subjected to no aeration, 3 hours of aeration, or 24 hours of aeration prior to 9 days of fermentation. It was observed that following fermentation in the absence of an aerobic growth period there were low levels of γ-decalactone, below the odor threshold. However, when the strains were cultured aerobically for 24 hours prior to fermentation, the level of γ-decalactone produced was substantially increased. Further experimentation was performed to determine how the length of aerobic growth affected γ-decalactone production. As shown in FIG. 6, aerobic growth of the engineered strains for even 3 hours resulted in γ-decalactone levels above the odor threshold. These results were not dependent on expression of the AAT, as strains that did not express the AAT also produced levels of γ-decalactone that were above the odor threshold in wine (i.e., 35 μg/L).

TABLE 1
Exemplary S. cerevisiae strains

| Name | Genotype | Strain background |
|---|---|---|
| y465 | ADE2::pPGK1-LFAH12 | WLP001 |
| y467 | ADE2::pPGK1-CpFAH | WLP001 |
| y1070 | pPDC6::pPGK1-CpFAH, pENO2-OLE1 | Elegance |
| y1094 | pPDC6::pPGK1-CpFAH | Elegance |
| y1124 | pPDC6::pPGK1-CpFAH, pENO2-OLE1, | Elegance |
| | pHSP26-MpAAT1**N385D V62A | |
| y1185 | pPDC6::pTDH3-CpFAH, pENO2-OLE1, | Elegance |
| | pHSP26-MpAAT1**N385D V62A; | |
| | FIG2::pRPL18B-ADR1*S230A | |
| y1330 | pPDC6::pPGK1-HbFAH | Elegance |
| y1331 | pPDC6::pPGK1-PlFAH | Elegance |
| y1332 | pPDC6::pPGK1-RcFAH | Elegance |
| y1333 | pPDC6::pPGK1-LFAH12 | Elegance |
| y1341 | pPDC6::pTDH3-CpFAH, pENO2-OLE1, | Elegance |
| | pRPL18B-ADR1(S230A) | |

What is claimed is:

1. A genetically modified yeast cell (modified cell) comprising:
a heterologous gene encoding an enzyme having oleate 12-hydroxylase activity;
wherein the modified cell is capable of producing a fermented product having an increased level of γ-decalactone in the absence of fatty acid supplementation as compared to a level of γ-decalactone produced by a counterpart cell that does not comprise the enzyme having oleate 12-hydroxylae activity.

2. A genetically modified yeast cell (modified cell) comprising:
a heterologous gene encoding an enzyme having oleate 12-hydroxylase activity; wherein the modified cell is capable of producing a fermented product having a level of γ-decalactone greater than 35 μg/L in the absence of fatty acid supplementation.

3. The modified cell of claim 1 or 2, wherein the enzyme having oleate 12-hydroxylase activity is from Claviceps purpurea, Lesquerella fendleri, Hiptage benghalensis, Physaria lindheimeri, or Ricinus communis.

4. The modified cell of claim 3, wherein the enzyme having oleate 12-hydroxylase activity comprises a sequence having at least 90% sequence identity to the amino acid sequence forth in any one of SEQ ID NOs: 6 or 20-23.

5. The modified cell of claim 4, wherein the enzyme having oleate 12-hydroxylase activity comprises the amino acid sequence set forth in any one of SEQ ID NOs: 6 or 20-23.

6. The modified cell of any one of claims 1-5, wherein the enzyme having oleate 12-hydroxylase activity comprises a sequence having at least 90% sequence identity to the amino acid sequence forth in SEQ ID NOs: 6.

7. The modified cell of claim 6, wherein the enzyme having oleate 12-hydroxylase activity comprises the amino acid sequence set forth in SEQ ID NOs: 6.

8. The modified cell of any one of claims 1-7, further comprising a gene encoding a deregulated transcription factor that increases peroxisomal size and number and increases beta-oxidation as compared to a counterpart transcription factor that is not deregulated.

9. The modified cell of claim 8, wherein the deregulated transcription factor is ADR1, PIP2, OAF1, or OAF3.

10. The modified cell of claim 9, wherein the deregulated transcription factor is ADR1 and comprises a substitution mutation of serine at position 230.

11. The modified cell of claim 9 or 10, wherein the deregulated transcription factor comprises an amino acid sequence having at least 90% sequence identity to the amino acid sequence set forth in SEQ ID NO: 24.

12. The modified cell of claim 11, wherein the deregulated transcription factor comprises the amino acid sequence set forth in SEQ ID NO: 24.

13. The modified cell of any one of claims 8-12, wherein the gene encoding the deregulated transcription factor is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

14. The modified cell of any one of claims 1-13, further comprising a gene encoding an enzyme having acyl-CoA desaturase 1 (OLE1) activity and/or a heterologous gene encoding an enzyme having alcohol-O-acyltransferase (AAT) activity.

15. The modified cell of claim 14, wherein the enzyme having OLE1 activity is derived from Saccharomyces cerevisiae.

16. The modified cell of claim 14 or 15, wherein the enzyme having OLE1 activity comprises a sequence having at least 90% sequence identity to the amino acid sequence forth in SEQ ID NO: 7.

17. The modified cell of claim 16, wherein the enzyme having OLE1 activity comprises the amino acid sequence set forth in SEQ ID NO: 7.

18. The modified cell of any one of claims 14-17, wherein the gene encoding the enzyme having acyl-CoA desaturase 1 (OLE1) activity is a copy of an endogenous gene encoding the enzyme having OLE1 activity.

19. The modified cell of any one of claims 14-18, wherein the enzyme having AAT activity is from Prunus persica, Fragaria x ananassa, Solanum lycopersicum, Malus domestica, or Cucumis melo.

20. The modified cell of claim 19, wherein the enzyme having AAT activity comprises a sequence having at least 90% sequence identity to the amino acid sequence set forth in any one of SEQ ID NOs: 1-5 or 25.

21. The modified cell of claim 20, wherein the enzyme having AAT activity comprises the amino acid sequence set forth in any one of SEQ ID NOs: 1-5 or 25.

22. The modified cell of any one of claims 1-21, wherein the gene encoding the enzyme having oleate 12-hydroxylase activity is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

23. The modified cell of any one of claims 8-22, wherein the gene encoding the deregulated transcription factor, the gene encoding the enzyme having acyl-CoA desaturase 1 (OLE1) activity, and/or the gene encoding the enzyme having alcohol-O-acyltransferase (AAT) activity is operably linked to a promoter selected from the group consisting of pHEM13, pSPG1, pPRB1, pQCR10, pPGK1, pOLE1, pERG25, pHHF2, pTDH1, pTDH2, pTDH3, pENO2, pHSP26, and pRPL18b.

24. The modified cell of any one of claims 1-23, wherein the yeast cell is of the genus Saccharomyces.

25. The modified cell of claim 24, wherein the yeast cell is of the species Saccharomyces cerevisiae (S. cerevisiae).

26. The modified cell of claim 25, wherein the yeast cell is S. cerevisiae California Ale Yeast strain WLP001, EC-1118, Elegance, Red Star Côte des Blancs, or Epernay II.

27. The modified cell of claim 24, wherein the yeast cell is of the species Saccharomyces pastorianus (S. pastorianus).

28. The modified cell of any one of claims 1-27, wherein the growth rate of the modified cell is not substantially impaired relative to a wild-type yeast cell that does not comprise the enzyme having oleate 12-hydroxylase activity.

29. The modified cell of any one of claims 1-28, wherein within one month of the start of fermentation, the modified cell ferments a comparable amount of fermentable sugar to the amount fermented by wild-type yeast cell that does not comprise the enzyme having oleate 12-hydroxylase activity.

30. The modified cell of claim 29, wherein within one month of the start of fermentation, the modified cell reduces the amount of fermentable sugars in a medium by at least 95%.

31. The modified cell of any one of claims 1-30, wherein within one month of the start of fermentation, the modified cell ferments a comparable amount of fermentable sugar to the amount fermented by wild-type yeast cell that does not comprise the enzyme having oleate 12-hydroxylase activity under anaerobic or semi-anaerobic conditions.

32. A genetically modified yeast cell (modified cell) comprising:
two or more genes, wherein the two or more genes are selected from the group consisting of:
a first heterologous gene encoding an enzyme having alcohol-O-acyltransferase (AAT) activity,
a second heterologous gene encoding an enzyme having fatty acid hydroxylase (FAH) activity, and
a gene encoding an enzyme having acyl-CoA desaturase 1 (OLE1) activity.

33. The modified cell of claim 32, wherein the two or more genes comprise the first heterologous gene encoding the enzyme having alcohol-O-acyltransferase (AAT) activity and the second heterologous gene encoding the enzyme having fatty acid hydroxylase (FAH) activity.

34. The modified cell of claim 32, wherein the two or more genes comprise the first heterologous gene encoding the enzyme having alcohol-O-acyltransferase (AAT) activity and the gene encoding the enzyme having acyl-CoA desaturase 1 (OLE1) activity.

35. The modified cell of claim 32, wherein the two or more genes comprise the second heterologous gene encoding the enzyme having fatty acid hydroxylase (FAH) activity and the gene encoding the enzyme having acyl-CoA desaturase 1 (OLE1) activity.

36. The modified cell of claim 32, wherein the two or more genes comprise the first heterologous gene encoding the enzyme having alcohol-O-acyltransferase (AAT) activity, the second heterologous gene encoding the enzyme having fatty acid hydroxylase (FAH) activity, and the gene encoding the enzyme having acyl-CoA desaturase 1 (OLE1) activity.

37. The modified cell of any one of claims 32-34 or 36, wherein the enzyme having AAT activity is derived from Prunus persica, Fragaria x ananassa, Solanum lycopersicum, Malus domestica, or Cucumis melo.

38. The modified cell of any one of claims 32-34, 36 or 37, wherein the enzyme having AAT activity comprises a sequence having at least 90% sequence identity to the amino acid sequence set forth in any one of SEQ ID NOs: 1-5 or 25.

39. The modified cell of claim 38, wherein the enzyme having AAT activity comprises the amino acid sequence set forth in any one of SEQ ID NOs: 1-5 or 25.

40. The modified cell of claim 39, wherein the enzyme having AAT activity comprises the amino acid sequence set forth in SEQ ID NO: 1.

41. The modified cell of any one of claims 32, 33, or 35-40, wherein the enzyme having FAH activity is derived from Claviceps purpurea.

42. The modified cell of any one of claims 32, 33, or 35-41, wherein the enzyme having FAH activity comprises a sequence having at least 90% sequence identity to the amino acid sequence forth in SEQ ID NO: 6 or 20-23.

43. The modified cell of claim 42, wherein the enzyme having FAH activity comprises the amino acid sequence set forth in SEQ ID NO: 6 or 20-23.

44. The modified cell of any one of claim 32 or 34-43, wherein the enzyme having OLE1 activity is derived from Saccharomyces cerevisiae.

45. The modified cell of any one of claim 32 or 34-44, wherein the enzyme having OLE1 activity comprises a sequence having at least 90% sequence identity to the amino acid sequence forth in SEQ ID NO: 7.

46. The modified cell of claim 45, wherein the enzyme having OLE1 activity comprises the amino acid sequence set forth in SEQ ID NO: 7.

47. The modified cell of any one of claim 32 or 34-46, wherein the gene encoding the enzyme having acyl-CoA desaturase 1 (OLE1) activity is a copy of an endogenous gene encoding the enzyme having OLE1 activity.

48. The modified cell of any one of claims 32-47, wherein each of the genes is operably linked to a promoter selected from the group consisting of pHEM13, pSPG1, pPRB1, pQCR10, pPGK1, pOLE1, pERG25, pHHF2, pTDH1, pTDH2, pTDH3, pENO2, and pHSP26.

49. The modified cell of any one of claims 32-48, wherein at least one of the genes encodes a localization signal linked to the enzyme.

50. The modified cell of claim 49, wherein the enzyme having AAT activity comprises a localization signal.

51. The modified cell of claim 50, wherein the localization signal is a peroxisome targeting signal.

52. The modified cell of any one of claims 32-51, wherein the yeast cell is of the genus Saccharomyces.

53. The modified cell of claim 52, wherein the yeast cell is of the species Saccharomyces cerevisiae (S. cerevisiae).

54. The modified cell of claim 53, wherein the yeast cell is S. cerevisiae California Ale Yeast strain WLP001, EC-1118, Elegance, Red Star Côte des Blancs, or Epernay II.

55. The modified cell of claim 52, wherein the yeast cell is of the species Saccharomyces pastorianus (S. pastorianus).

56. The modified cell of any one of claims 32-55, wherein growth rate of the modified cell is not substantially impaired relative to a wild-type yeast cell that does not comprise the first heterologous gene, the second heterologous gene, and the third gene.

57. The modified cell of claim 56, wherein within one month of the start of fermentation, the modified cell ferments a comparable amount of fermentable sugar to the amount fermented by wild-type yeast cell that does not comprise the first heterologous gene, the second heterologous gene, and/or the third gene.

58. The modified cell of claim 57, wherein within one month of the start of fermentation, the modified cell reduces the amount of fermentable sugars in a medium by at least 95%.

59. The modified cell of any one of claims 32-58, wherein within one month of the start of fermentation, the modified cell ferments a comparable amount of fermentable sugar to the amount fermented by wild-type yeast cell that does not comprise the first heterologous gene, the second heterologous gene, and the third heterologous under anaerobic or semi-anaerobic conditions.

60. The modified cell of any one of claims 32-59, further comprising a deregulated transcription factor that increases peroxisomal size and number and increases and beta-oxidation.

61. The modified cell of claim 60, wherein the deregulated transcription factor is ADR1, PIP2, OAF1, or OAF3.

62. The modified cell of claim 61, wherein the deregulated transcription factor is ADR1 and comprises a substitution mutation of serine at position 230.

63. A method of producing a fermented product comprising,
contacting the modified cell of any one of claims 1-62 with a medium comprising at least one fermentable sugar,
wherein the contacting is performed during at least a first fermentation process, to produce a fermented product.

64. The method of claim 63, wherein the medium does not comprise supplemented fatty acids.

65. The method of claim 64, wherein the medium does not comprise supplemented oleic acid and/or ricinoleic acid.

66. The method of any one of claims 63-65, wherein at least one fermentable sugar is provided in at least one sugar source.

67. The method of any one of claims 63-66, wherein the fermentable sugar is glucose, fructose, sucrose, maltose, and/or maltotriose.

68. The method of any one of claims 63-67, wherein the fermented product comprises an increased level of at least one desired product as compared to a fermented product produced by a counterpart cell that does not express the heterologous gene encoding the enzyme having oleate 12-hydroxylase activity.

69. The method of claim 68, wherein the desired product is γ-decalactone.

70. The method of any one of claims 63-69, wherein the fermented product comprises a reduced level of at least one undesired product as compared to a fermented product produced by a counterpart cell that does not express the heterologous gene encoding the enzyme having oleate 12-hydroxylase activity.

71. The method of claim 70, wherein the at least one undesired product is ethyl acetate.

72. The method of any one of claims 63-71, wherein the fermented product is a fermented beverage.

73. The method of claim 72, wherein the fermented beverage is beer, wine, sparkling wine (champagne), wine cooler, wine spritzer, hard seltzer, sake, mead, kombucha, or cider.

74. The method of any one of claims 63-73, wherein the sugar source comprises wort, must, fruit juice, honey, rice starch, or a combination thereof.

75. The method of claim 74, wherein the sugar source is pre-oxygenated prior to the first fermentation process.

76. The method of any one of claims 63-75, wherein the first fermentation process comprises aeration for a period of time.

77. The method of claim 76, wherein the period of time is at least 3 hours.

78. The method of any one of claims 74-77, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

79. The method of any one of claims 74-77, wherein the sugar source is wort and the method further comprises producing the medium, wherein producing the medium comprises:
(a) contacting a plurality of grains with water; and
(b) boiling or steeping the water and grains to produce wort.

80. The method of claim 79, further comprising adding at least one hop variety to the wort to produce a hopped wort.

81. The method of claim 79 or 80, further comprising adding at least one hop variety to the medium.

82. The method of any one of claims 74-77, wherein the sugar source is must and the method further comprises producing the medium, wherein producing the medium comprises crushing a plurality of fruit to produce the must.

83. The method of claim 82, further comprising removing solid fruit material from the must to produce a fruit juice.

84. The method of any one of claims 63-83, further comprising at least one additional fermentation process.

85. The method of any one of claims 63-84, further comprising carbonating the fermented product.

86. A fermented product produced, obtained, or obtainable by the method of any one of claims 63-85.

87. A method of producing a composition comprising ethanol, the method comprising
contacting the modified cell of any one of claims 1-62 with a medium comprising at least one fermentable sugar,
wherein the contacting is performed during at least a first fermentation process, to produce the composition comprising ethanol.

88. The method of claim 87, wherein at least one fermentable sugar is provided in at least one sugar source.

89. The method of claim 87 or 88, wherein the fermentable sugar is glucose, fructose, sucrose, maltose, and/or maltotriose.

90. The method of any one of claims 87-89, wherein the composition comprising ethanol comprises an increased level of at least one desired product as compared to a composition comprising ethanol produced by a counterpart cell that does not express the heterologous gene encoding an enzyme having oleate 12-hydroxylase activity or a counterpart cell that expresses a wild-type enzyme having oleate 12-hydroxylase activity.

91. The method of claim 90, wherein the desired product is γ-decalactone.

92. The method of any one of claims 87-91, wherein the composition comprising ethanol comprises a reduced level of at least one undesired product as compared to a composition comprising ethanol produced by a counterpart cell that does not express the heterologous gene encoding an enzyme having oleate 12-hydroxylase activity or a counterpart cell that expresses a wild-type enzyme having oleate 12-hydroxylase activity.

93. The method of claim 92, wherein the at least one undesired product is ethyl acetate.

94. The method of any one of claims 87-93, wherein the composition comprising ethanol is a fermented beverage.

95. The method of claim 94, wherein the fermented beverage is beer, wine, sparkling wine (champagne), wine cooler, wine spritzer, hard seltzer, sake, mead, kombucha, or cider.

96. The method of any one of claims 88-95, wherein the sugar source comprises wort, must, fruit juice, honey, rice starch, or a combination thereof.

97. The method of claim 96, wherein the fruit juice is a juice obtained from at least one fruit selected from the group consisting of grapes, apples, blueberries, blackberries, raspberries, currants, strawberries, cherries, pears, peaches, nectarines, oranges, pineapples, mangoes, and passionfruit.

98. The method of claim 96, wherein the sugar source is wort and the method further comprises producing the medium, wherein producing the medium comprises:
(a) contacting a plurality of grains with water; and
(b) boiling or steeping the water and grains to produce wort.

99. The method of claim 98, further comprising adding at least one hop variety to the wort to produce a hopped wort.

100. The method of any one of claims 87-99, further comprising adding at least one hop variety to the medium.

101. The method of claim 96, wherein the sugar source is must and the method further comprises producing the medium, wherein producing the medium comprises crushing a plurality of fruits to produce the must.

102. The method of claim 101, further comprising removing solid fruit material from the must to produce a fruit juice.

103. The method of any one of claims 87-102, further comprising at least one additional fermentation process.

104. The method of any one of claims 87-103, further comprising carbonating the composition comprising ethanol.

105. A composition comprising ethanol produced, obtained, or obtainable by the method of any one of claims 87-104.


SEQUENCE LISTING (25 sequences)

SEQ ID NO: 1 - protein, 450 aa, Prunus persica
MGSLCPLLFPVNRFEPELITPAKPTPIETKQLSDIDDQDGLRFHFPVIISYKNNPSMKGNDAVMVIREALSRALVYYYPLAGRLREGPNRKLMVECNGEGVLFIEANADVTLEQLGDRILPPCPVLEEFLSNPPGSDGILGCPLLLVQVTRLTCGGFIFGLRINHAMCDAVGLAKFLNAIGEMAQGADSLSVPPVWARELLNARDPPTVTRWHYEYDQLLDSQGSFIAAIDQSNMAQRSFYFGPQQIRALRKHLPPHLSTCSSFELITACVWRCRTLSLRLNPKDTVRISCAVNARGKSINDLCLPSGFYGNAFSIPTAVSTVELLCASPLGYGVELVRKSKAQMDKEYMQSLADFFVIRGRPPLPMGWNVFIVSDNRHTGFGEFDVGWGRPLFAGLARAFSMISFYVRDNNQEEEFGTLVPICLPSTSLERFEEELKKMTLEHVEEISK

SEQ ID NO: 2 - protein, 453 aa, Fragaria x ananassa
MGEKIEVSINSKHTIKPSTSSTPLQPYKLTLLDQLTPPAYVPIVFFYPITDHDFNLPQTAADLRQALSETLTLYYPLSGRVKNNLYIDDFEEGVPYLEARVNCDMTDFLRLRKIECLNEFVPIKPFSMEAISDERYPLLGVQVNVFDSGIAIGVSVSHKLIDGGTADCFLKSWGAVFRGCRENIIHPSLSEAALLFPPRDDLPEKYVDQMEALWFAGKKVATRRFVFGVKAISSIQDEAKSESVPKPSRVHAVTGFLWKHLIAASRALTSGTTSTRLSIAAQAVNLRTRMNMETVLDNATGNLFWWAQAILELSHTTPEISDLKLCDLVNLLNGSVKQCNGDYFETFKGKEGYGRMCEYLDFQRTMSSMEPAPDIYLFSSWTNFFNPLDFGWGRTSWIGVAGKIESASCKFIILVPTQCGSGIEAWVNLEEEKMAMLEQDPHFLALASPKTLI

SEQ ID NO: 3 - protein, 442 aa, Solanum lycopersicum
MANTLPISINYHKPKLVVPSSVTPHETKRLSEIDDQGFIRFQIPILMFYKYNSSMKGKDPARIIEDGLSKTLVFYHPLAGRLIEGPNKKLMVNCNGEGVLFIEGDANIELEKLGESIKPPCPYLDLLLHNVPGSDGIIGSPLLLIQVTRFTCGGFAVGFRVSHTMMDGYGFKMFLNALSELIQGASTPSILPVWQRHLLSARSSPCITCSHHEFDEEIESKIAWESMEDKLIQESFFFGNEEMEVIKNQIPPNYGCTKFELLMAFLWKCRTIALDLHPEEIVRLTYVINIRGKKSLNIELPIGYYGNAFVTPVVVSKAGLLCSNPVTYAVELIKKVKDHINEEYIKSVIDLTVIKGRPELTKSWNFLVSDNRYIGFDEFDFGWGNPIFGGISKATSFISFGVSVKNDKGEKGVLIAISLPPLAMKKLQDIYNMTFRVIIPRI

SEQ ID NO: 4 - protein, 455 aa, Malus domestica
MMSFSVLQVKRLQPELITPAKSTPQETKFLSDIDDQESLRVQIPIIMCYKDNPSLNKNRNPVKAIREALSRALVYYYPLAGRLREGPNRKLVVDCNGEGILFVEASADVTLEQLGDKILPPCPLLEEFLYNFPGSDGIIDCPLLLIQVTCLTCGGFILALRLNHTMCDAAGLLLFLTAIAEMARGAHAPSILPVWERELLFARDPPRITCAHHEYEDVIGHSDGSYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRVGFGDVNFGWGQPVFAGPVKALDLISFYVQHKNNTEDGILVPMCLPSSAMERFQQELERITQEPKEDICNNLRSTSQ

SEQ ID NO: 5 - protein, 462 aa, Cucumis melo
METMQTIDFSFHVRKCQPELIAPANPTPYEFKQLSDVDDQQSLRLQLPFVNIYPHNPSLEGRDPVKVIKEAIGKALVFYYPLAGRLREGPGRKLFVECTGEGILFIEADADVSLEEFWDTLPYSLSSMQNNIIHNALNSDEVLNSPLLLIQVTRLKCGGFIFGLCFNHTMADGFGIVQFMKATAEIARGAFAPSILPVWQRALLTARDPPRITFRHYEYDQVVDMKSGLIPVNSKIDQLFFFSQLQISTLRQTLPAHLHDCPSFEVLTAYVWRLRTIALQFKPEEEVRFLCVMNLRSKIDIPLGYYGNAVVVPAVITTAAKLCGNPLGYAVDLIRKAKAKATMEYIKSTVDLMVIKGRPYFTVVGSFMMSDLTRIGVENVDFGWGKAIFGGPTTTGARITRGLVSFCVPFMNRNGEKGTALSLCLPPPAMERFRANVHASLQVKQVVDAVDSHMQTIQSASK

SEQ ID NO: 6 - protein, 477 aa, Claviceps purpurea
MASATPAMSENAVLRHKAASTTGIDYESSAAVSPAESPRTSASSTSLSSLSSLDANEKKDEYAGLLDTYGNAFTPPDFSIKDIRAAIPKHCYERSTIKSYAYVLRDLLCLSTTFYLFHNFVTPENIPSNPLRFVLWSIYTVLQGLFATGLWVIGHECGHCAFSPSPFISDLTGWVIHSALLVPYFSWKFSHSAHHKGIGNMERDMVFLPRTREQQATRLGRAVEELGDLCEETPIYTALHLVGKQLIGWPSYLMTNATGHNFHERQREGRGKGKKNGFGGGVNHFDPRSPIFEARQAKYIVLSDIGLGLAIAALVYLGNRFGWANMAVWYFLPYLWVNHWLVAITFLQHTDPTLPHYNREEWNFVRGGACTIDRDLGFIGRHLFHGIADTHVVHHYVSRIPFYNADEASEAIKPIMGKHYRSDTAHGPVGFLHALWKTARWCQWVEPSADAQGAGKGILFYRNRNKLGTKPISMKTQ

SEQ ID NO: 7 - protein, 510 aa, Saccharomyces cerevisiae
MPTSGTTIELIDDQFPKDDSASSGIVDEVDLTEANILATGLNKKAPRIVNGFGSLMGSKEMVSVEFDKKGNEKKSNLDRLLEKDNQEKEEAKTKIHISEQPWTLNNWHQHLNWLNMVLVCGMPMIGWYFALSGKVPLHLNVFLFSVFYYAVGGVSITAGYHRLWSHRSYSAHWPLRLFYAIFGCASVEGSAKWWGHSHRIHHRYTDTLRDPYDARRGLWYSHMGWMLLKPNPKYKARADITDMTDDWTIRFQHRHYILLMLLTAFVIPTLICGYFFNDYMGGLIYAGFIRVFVIQQATFCINSMAHYIGTQPFDDRRTPRDNWITAIVTFGEGYHNFHHEFPTDYRNAIKWYQYDPTKVIIYLTSLVGLAYDLKKFSQNAIEEALIQQEQKKINKKKAKINWGPVLTDLPMWDKQTFLAKSKENKGLVIISGIVHDVSGYISEHPGGETLIKTALGKDATKAFSGGVYRHSNAAQNVLADMRVAVIKESKNSAIRMASKRGEIYETGKFF

SEQ ID NO: 8 - DNA, 801 bp, Saccharomyces cerevisiae
taatgtagaaggttgagaacaaccggatcttgcggtcatttttcttttcgaggaaagtgcaagtctgccactttccagaaggcatagccttgcccttttgttgatatttctccccaccgtaattgttgcattcgcgatcttttcaacaatacattttatcatcaagcccgcaaatcctctggagtttgtcctctcgttcactgttgggaaaaacaatacgcctaattcgtgattaagattcttcaaaccatttcctgcggagtttttactgtgtgttgaacggttcacagcgtaaaaaaaagttactataggcacggtattttaatttcaattgtttagaaagtgccttcacaccattagcccctgggattaccgtcataggcactttctgctgagctcctgcgagatttctgcgctgaaagagtaaaagaaatctttcacagcggctccgcgggcccttctacttttaaacgagtcgcaggaacagaagccaaatttcaaagaacgctacgctttcgccttttctggttctcccaccaataacgctccagcttgaacaaagcataagactgcaaccaaagcgctgacggacgatccgaagataaagcttgctttgcccattgttctcgtttcgaaaggctatataaggacacggattttccttttttttttccacctattgtctttctttgttaagcttttattctccgggttttttttttttgagcatatcaaaagctttcttttcgcaaatcaaacatagcaaaccgaactcttcgaacacaattaaatacacataaa

SEQ ID NO: 9 - DNA, 750 bp, Saccharomyces cerevisiae
atgaagttcacttcacatccaatgagaaaaacaaaatccgcagggctatcacccagaacatcctccacttcatcttcttcaggacagagaaaagcgcatcaccaccaccatcaccacaaccacgtttcaaggacgaaaactaccgaaagcaccaaatcaggcaacagcaaaaaggacagttcctcatcctcaacaaacgaccatcaatttaaaaggtctgaaaagaagaaaaaaagtaaatttggctcgatcttcaaaaaagttttcggatgaaccggattaatacaagtaaaatcagcaaagatatagaagacaaaataagcgtgaaaacaatcataaaccactcacaacgggggttttcagctgttactcctccatacatacattttgataaagatataatgttatatttcttttcgtaattttgttttacttcggtttgctctatagatttcatcagccgcaccgaaaagggagatcaataaggtaccctttaaaagggataagaagcctaacatcaccccaataaatggagtaatggccagcattggatgaagagaagaattacgggatactgggataacactgttaaaaatgcttcgcgacgtgagggtcttatataaattgaactgccaaatctctttcacattatccaggatagtttggaatgtgtgttactgaaagatcagaatcaataaatacaatcaatacaaatatttagcgcataaaattcaaacaaagtttactgaa

SEQ ID NO: 10 - DNA, 750 bp, Saccharomyces cerevisiae
cgagaaacagggggggagaaaaggggaaaagagaaggaaagaaagactcatctatcgcagataagacaatcaaccctcatggcgcctccaaccaccatccgcactagggaccaagcgctcgcaccgttagcaacgcttgactcacaaaccaactgccggctgaaagagcttgtgcaatgggagtgccaattcaaaggagccgaatacgtctgttcgccttttaagaggctttttgaacactgcattgcacccgacaaatcagccactaactacgaggtcacggatacatataccaatagttaaaaaattacatatactctatatagcacagtagtgtgataaataaaaaattttgccaagacttttttaaactgcacccgacagatcaggtctgtgcctactatgcacttatgcccggggtcccgggaggagaaaaaacgagggctgggaaatgtccgtggacttaaaacgctccgggttagcagagtagcagggctttcggctttggaaatttaggtgacttgttgaaaaagcaaaatttgggctcagtaatgccacagcagtggcttatcacgccaggactgcgggagtggcgggggcaaacacacccgcgataaagagcgcgatgaatataaaagggggccaatgttacgtcccgttatattggagttcttcccatacaaacttaagagtccaattagcttcatcgccaataaaaaaacaaactaaacctaattctaacaagcaaag

SEQ ID NO: 11 - DNA, 696 bp, Saccharomyces cerevisiae
gagagctggccaaaaagagggccgaagacggcgttgaatttcattcaaaactatttagaagggcagagccaggtgaggatttagattattatatttacaagcacatccctgaagggaccgacaagcatgaagaacagatcaggagcattttggaaactgccccgattttaccaggacaggcattcactgaaaaattttctattccggcttataaaaagcatggaatccaaaagaattaggcttctcattctattttaattatactagtacgatttctcactctgtaatttaatatcagtgtaatatgcacctagttatgggtagtttttgctaacgttacgagccgcgaaactgtcctcaatcttcaccactacctctaatgactgaagaatgctatgcgatataacgctgccgcactttgaatatatacttatatttacatagttttcaagtgcgtattactattgcaaagtagtattttgtcacgtgattttgatccaattaaaactaaatatggttcaacccgttgtttccgcatcaaaaaaccataccatttatcaaggggacgggatatatcacataacagtttgaatgcataatttgttatagatatcttctggaataatcttcacagcaaaagcgcaagtcgaataatatatcgataaatacaatccataagacttaaaactaacctca

SEQ ID NO: 12 - DNA, 813 bp, Saccharomyces cerevisiae
gcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaa

SEQ ID NO: 13 - DNA, 800 bp, Saccharomyces cerevisiae
ctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaa

SEQ ID NO: 14 - DNA, 680 bp, Saccharomyces cerevisiae
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 15 - DNA, 699 bp, Saccharomyces cerevisiae
attgaatacattagcaacgcgtccagcatttttcggaagtgtctcataaactttactcaagagttaagtactgaaaaattcgacttttatgatagttcaagtgtcgacgctgcgggtatagaaagggttctttactctatagtgcctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaagcattatcttcctaccagagttgattgttaaaaacgtatttatagcaaacgcaattgtaattaattcttattttgtatcttttcttcccttgtctcaatcttttatttttattttattcttcttttcttagtttctttcataacaccaagcaactaatactataacatacaataata

SEQ ID NO: 16 - DNA, 800 bp, Saccharomyces cerevisiae
caatattctgcgcacatcaatcattttcttactacatacactaacattactcctagtttaatttaattgaatttttaactttcttttcttttcatttggcaatttggctccttgaaaacaagactatgggtctgtctcataagcctcagggggggaccccaaaaaaataacgcggccatcttgcatgcaccgttgaacctgtagcttacagtaagccacaattctcttaccttcttggcaatgtggcacaaaataatctggttatgtgtcttcatttggtaatcactgggatgttactggggcagcagcaactccgtgtgtacccctaactccgtgtgtacccctaaagaaccttgcctgtcaaggtgcattgttggatcggaatagtaaccgtctttacatgaacatccacaaccaacgaaagtgctttttcaagcattgcttgatttctagaaagatcgatggttattccctcccccttatgcgtccaaaaatatagggtgctcgtaacagtaaggtattcgcacttagcgtgctcgcaacacaaaattaagtaatatgcgagttttagatgtccttgcggatctatgcacgttcttgagtggtatttcataacaacggttctttttcacccttattcctaaacatataaataggacctccattagttagagatctgtttttaatccattcacctttcattctactctcttatactaataaaaccaccgataaagatatatcagatctctattaaaacaggtatccaaaaaagcaaacaaacaaactaaacaaattaa

SEQ ID NO: 17 - protein, 3 aa, Artificial Sequence
  Synthetic
SKL

SEQ ID NO: 18 - protein, 11 aa, Artificial Sequence
  Synthetic
GSLGRGRRSKL

SEQ ID NO: 19 - DNA, 625 bp, Saccharomyces cerevisiae
ccgtaaaccttggcgctttccttgggaagtattcaagaagtgccttgtccggtttctgtggctcacaaaccagcgcgcccgatatggctttcttttcacttatgaatgtaccagtacgggacaattagaacgctcctgtaacaatctctttgcaaatgtggggttacattctaaccatgtcacactgctgacgaaattcaaagtaaaaaaaaatgggaccacgtcttgagaacgatagattttctttattttacattgaacagtcgttgtctcagcgcgctttatgttttcattcatacttcatattataaaataacaaaagaagaatttcatattcacgcccaagaaatcaggctgctttccaaatgcaattgacacttcattagccatcacacaaaactctttcttgctggagcttcttttaaaaaagacctcagtacaccaaacacgttacccgacctcgttattttacgacaactatgataaaattctgaagaaaaaataaaaaaattttcatacttcttgcttttatttaaaccattgaatgatttcttttgaacaaaactacctgtttcaccaaaggaaatagaaagaaaaaatcaattagaagaaaacaaaaaacaaa

SEQ ID NO: 20 - protein, 383 aa, Hiptage benghalensis
MGAGGRMPTSVSKGQGMENEVKHGPCEKPPFTVGQLKRAIPPHCFERSLIRSSSYLLRDLFFVFVFYYVATSYFHLLPYPFNYAAWPIYWGFQGCALTGIWVLGHECGHHAFSDYQLVDDIVGLIIHTALLVPYFSWKISHRRHHSNTGSLEREEVFAPKPKAEIQWYLKHLNNPPGRAIVLLNTLLLGWPLYVAFNVAGRRYDRFACHFDPYSPIFSASERHLIYITDAGIYATTFILYRAAAAKGLTWLICVYGVPLVIVNAFLVLVTYLQHTHPVLPHYDNSEWDWLRGALVTVDRDYGILNEVFHHIADTHVAHHLFSKIPQYHGMEATKAIKPILGEYYQFDGTPFLKALWREARECVYVDRDEGDPKRGVYWYGNKF

SEQ ID NO: 21 - protein, 383 aa, Physaria lindheimeri
MGAGGRIMVTPSSKKSKPEALRRGPGEKPPFTVQDLRKAIPRHCFKRSIPRSFSYLLTDIILASCFYYVATNYFSLLPQPLSTYFAWPLYWVCQGCVLTGVWVLGHECGHQAFSDYQWVDDTVGFIIHTFLLVPYFSWKYSHRRHHANNGSLERDEVFVPPKKAAVKWYVKYLNNPLGRTVVLIVQFVLGWPLYLAFNVSGRSYDGFASHFFPHAPIFKDRERLHIYITDAGILAVCYGLYRYAATKGLTAMIYVYGVPLLVVNFFLVLVTFLQHTHPSLPHYDSTEWDWIRGAMVTVDRDYGILNKVFHNITDTHVAHHLFATIPHYNAMEATEAIKPILGDYYHFDGTPWYVAMYREAKQCLYVEQDTEKKKGVYYYNNKL

SEQ ID NO: 22 - protein, 387 aa, Ricinus communis
MGGGGRMSTVITSNNSEKKGGSSHLKRAPHTKPPFTLGDLKRAIPPHCFERSFVRSFSYVAYDVCLSFLFYSIATNFFPYISSPLSYVAWLVYWLFQGCILTGLWVIGHECGHHAFSEYQLADDIVGLIVHSALLVPYFSWKYSHRRHHSNIGSLERDEVFVPKSKSKISWYSKYSNNPPGRVLTLAATLLLGWPLYLAFNVSGRPYDRFACHYDPYGPIFSERERLQIYIADLGIFATTFVLYQATMAKGLAWVMRIYGVPLLIVNCFLVMITYLQHTHPAIPRYGSSEWDWLRGAMVTVDRDYGVLNKVFHNIADTHVAHHLFATVPHYHAMEATKAIKPIMGEYYRYDGTPFYKALWREAKECLFVEPDEGAPTQGVFWYRNKY

SEQ ID NO: 23 - protein, 384 aa, Lesquerella fenderia
MGAGGRIMVTPSSKKSETEALKRGPCEKPPFTVKDLKKAIPQHCFKRSIPRSFSYLLTDITLVSCFYYVATNYFSLLPQPLSTYLAWPLYWVCQGCVLTGIWVIGHECGHHAFSDYQWVDDTVGFIFHSFLLVPYFSWKYSHRRHHSNNGSLEKDEVFVPPKKAAVKWYVKYLNNPLGRILVLTVQFILGWPLYLAFNVSGRPYDGFASHFFPHAPIFKDRERLQIYISDAGILAVCYGLYRYAASQGLTAMICVYGVPLLIVNFFLVLVTFLQHTHPSLPHYDSTEWEWIRGALVTVDRDYGILNKVFHNITDTHVAHHLFATIPHYNAMEATEAIKPILGDYYHFDGTPWYVAMYREAKECLYVEPDTERGKKGVYYYNNKL

SEQ ID NO: 24 - protein, 1323 aa, Saccharomyces cerevisiae
MANVEKPNDCSGFPVVDLNSCFSNGFNNGKQEIEMETDDSPILLMSSSASRENSNTFSVIQRTPDGKIITTNNNMNSKINKQLDKLPENLRLNGRTPSGKLRSFVCEVCTRAFARQEHLKRHYRSHTNEKPYPCGLCNRCFTRRDLLIRHAQKIHSGNLGETISHTKKVSRTITKARKNSASSVKFQTPTYGTPDNGNFLNRTTANTRRKASPEANVKRKYLKKLTRRAAFSAQSASSYALPDQSSLEQHPKDRVKFSTPELVPLDLKNPELDSSFDLNMNLDLNLNLDSNFNIALNRSDSSGSTMNLDYKLPESANNYTYSSGSPTRAYVGANTNSKNASFSDADLLSSSYWIKAYNDHLFSVSESDETSPMNSELNDTKLIVPDFKSTIHHLKDSRSSSWTVAIDNNSNNNKVSDNQPDFVDFQELLDNDTLGNDLLETTAVLKEFELLHDDSVSATATSNEIDLSHLNLSNSPISPHKLIYKNKEGTNDDMLISFGLDHPSNREDDLDKLCNMTRDVQAIFSQYLKGEESKRSLEDFLSTSNRKEKPDSGNYTFYGLDCLTLSKISRALPASTVNNKQPSHSIESKLFNEPMRNMCIKVLRYYEKFSHDSSESVMDSNPNLLSKELLMPAVSELNEYLDLFKNNFLPHFPIIHPSLLDLDLDSLQRYTNEDGYDDAENAQLFDRLSQGTDKEYDYEHYQILSISKIVCLPLFMATFGSLHKFGYKSQTIELYEMSRRILHSFLETKRRCRSTTVNDNYQNIWLMQSLILSFMFALVADYLEKIDSSLMKRQLSALCSTIRSNCLPTISANSEKSINNNNEPLTFGSPLQYIIFESKIRCTLMAYDFCQFLKCFFHIKFDLSIKEKDVETIYIPDNESKWASESIICNGHVVQKQNFYDFRNFYYSFTYGHLHSIPEFLGSSMIYYEYDLRKGTKSHVFLDRIDTKRLERSLDTSSYGNDNMAATNKNIAILIDDTIILKNNLMSMRFIKQIDRSFTEKVRKGQIAKIYDSFLNSARLNFLKNYSVEVLCEFLVALNFSIRNISSLYVEEESDCSQRMNSPELPRIHLNNQALSVFNLQGYYYCFILIIKFLLDFEATPNFKLLRIFIELRSLANSILLPTLSRLYPQEFSGFPDVVFTQQFINKDNGMLVPGLSANEHHNGASAAVKTKLAKKINVEGLAMFINEILVNSFNDTSFLNMEDPIRNEFSFDNGDRAVTDLPRSAHFLSDTGLEGINFSGLNDSHQTVSTLNLLRYGENHSSKHKNGGKGQGFAEKYQLSLKYVTIAKLFFTNVKENYIHCHMLDKMASDFHTLENHLKGNS

SEQ ID NO: 25 - protein, 455 aa, Artificial Sequence
  Synthetic
MMSFSVLQVKRLQPELITPAKSTPQETKFLSDIDDQESLRVQIPIIMCYKDNPSLNKNRNPAKAIREALSRALVYYYPLAGRLREGPNRKLVVDCNGEGILFVEASADVTLEQLGDKILPPCPLLEEFLYNFPGSDGIIDCPLLLIQVTCLTCGGFILALRLNHTMCDAAGLLLFLTAIAEMARGAHAPSILPVWERELLFARDPPRITCAHHEYEDVIGHSDGSYASSNQSNMVQRSFYFGAKEMRVLRKQIPPHLISTCSTFDLITACLWKCRTLALNINPKEAVRVSCIVNARGKHNNVRLPLGYYGNAFAFPAAISKAEPLCKNPLGYALELVKKAKATMNEEYLRSVADLLVLRGRPQYSSTGSYLIVSDNTRVGFGDVDFGWGQPVFAGPVKALDLISFYVQHKNNTEDGILVPMCLPSSAMERFQQELERITQEPKEDICNNLRSTSQ